(19) United States
(12) Patent Application Publication
(10) Pub. No.: US 2005/0120393 A1
(43) Pub. Date: Jun. 2, 2005

(54) Full-length prostate selective polynucleotides and polypeptides

(75) Inventors: Zairen Sun, Rockville, MD (US); Xuan Li, Silver Spring, MD (US); Gilbert Jay, North Bethesda, MD (US); Karl F. Kovacs, Rockville, MD (US); Youmin Shu, Potomac, MD (US); Wufang Fan, Germantown, MD (US)

Correspondence Address: MILLEN, WHITE, ZELANO & BRANIGAN, P.C., 2200 CLARENDON BLVD., SUITE 1400, ARLINGTON, VA, 22201, US

(73) Assignee: OriGene Technologies, Inc., Rockville, MD (US)

(21) Appl. No.: 10/485,555

(22) PCT Filed: Aug. 2, 2002
(86) PCT No.: PCT/US02/24431

Related U.S. Application Data
(60) Provisional application No. 60/309,470, filed on Aug. 3, 2001.
(60) Provisional application No. 60/330,747, filed on Oct. 30, 2001.

(51) Int. Cl. A01K 67/00; G06F 17/60; C12Q 1/68; C07H 21/04; C07K 14/47

(52) U.S. Cl. 800/8; 435/6; 435/69.1; 435/320.1; 435/325; 530/350; 536/23.2; 705/1

(57) ABSTRACT

The present invention relates to all facets of novel polynucleotides, the polypeptides they encode, antibodies and specific binding partners thereto, and their applications to research, diagnosis, drug discovery, therapy, clinical medicine, forensic science, pathology, and medicine, etc. The polynucleotides are expressed in prostate and are therefore useful in variety of ways, including, but not limited to, as molecular markers, as drug targets, and for detecting, diagnosing, staging, monitoring, prognosticating, preventing or treating, determining predisposition to, etc., diseases and conditions relating to prostate, such as prostate cancers, as well as other disease and conditions.

This application claims the benefit of U.S. Provisional Application Ser. Nos. 60/309,470, filed Aug. 3, 2001, and 60/330,747, filed Oct. 30, 2001, which are hereby incorporated by reference in their entirety.

### DESCRIPTION OF THE DRAWINGS

SEQ ID NOS 1-58 and 67-72 show the amino acid and nucleotide sequences of prostate-selective polynucleotides. The polynucleotides are human cDNAs.

FIGS. 1(A and B) shows a sequence comparison between PR51a and NM_{—}024080 (SEQ ID NO. 59).

FIG. 2 shows a sequence comparison between PR104, BC000623 (SEQ ID NO 65), and NM_{—}017713 (SEQ ID NO 66).

FIG. 3 shows a sequence comparison between PR155, NM_{—}015640 (SEQ ID NO. 63), and BC002488 (SEQ ID NO 64).

FIG. 4 shows a sequence comparison between PR376 and NM_{—}006515 (SEQ ID NO. 62).

FIG. 5 shows a sequence comparison between PR413 and AL050197 (SEQ ID NO. 60).

FIGS. 6(A and B) shows a sequence comparison between PR471 and NM_{—}024790 and (SEQ ID NO. 61).

### DESCRIPTION OF THE INVENTION

The present invention relates to all facets of novel polynucleotides, the polypeptides they encode, antibodies and specific binding partners thereto, and their applications to research, diagnosis, drug discovery, therapy, clinical medicine, forensic science and medicine, etc. The polynucleotides are expressed in prostate and are therefore useful in variety of ways, including, but not limited to, as molecular markers, as drug targets, and for detecting, diagnosing, staging, monitoring, prognosticating, preventing or treating, determining predisposition to, etc., diseases and conditions, especially relating to prostate, such as prostate cancer. The identification of specific genes, and groups of genes, expressed in pathways physiologically relevant to prostate permits the definition of functional and disease pathways, and the delineation of targets in these pathways which are useful in diagnostic, therapeutic, and clinical applications. The present invention also relates to methods of using the polynucleotides and related products (proteins, antibodies, etc.) in business and computer-related methods, e.g., advertising, displaying, offering, selling, etc., such products for sale, commercial use, licensing, etc.

The prostate is a secretory organ surrounding the neck of the bladder and urethra. Its primary function is to produce fluids and other materials necessary for sperm transport and maintenance. Structurally, it has both glandular and nonglandular components. The glandular component is predominantly comprised of ducts and acini responsible for the production and transport prostatic fluids. Epithelial cells are the main identifiable cell found in these regions, primarily of the basal and secretory types, but also endocrine-paracrine and transitional epithelial. The non-glandular component contains the capsular and muscle tissues, which, respectively, hold the organ together and function in fluid discharge. See, e.g., Histology for Pathologists, Sternberg, S. S., editor, Raven Press, N.Y., 1992, Chapter 40.

The major diseases of the prostate include, e.g., prostatic hyperplasia (BPH), prostatitis, and prostate cancer (e.g., prostatic adenocarcinoma). BPH is a benign, proliferative disease of the prostatic epithelial cells. While it may cause urinary tract obstruction in some patients, for the most part, it is generally asymptomatic. Prostate cancer, on the other hand, is the most common form of cancer in white males in the United States, occurring predominantly in males over age 50. The prevalence of prostate diseases, such as prostate cancer, has made the discovery of prostate selective markers and gene expression patterns of great importance.

The most common scale of assessing prostate pathology is the Gleason grading system. See, e.g., Bostwick, Am. J. Clin. Path., 102: s38-s56, 1994. Once the cancer is identified, staging can assess the size, location, and extent of the cancer. Several different staging scales are commonly used, including stages A-D, and Tumor-Nodes-Metastases (TNM). For treatment, diagnosis, staging, etc., of prostate conditions, methods can be carried out analogously to, and in combination with, U.S. Pat. Nos. 6,107,090; 6,057,116; 6,034,218; 6,004,267; 5,919,638; 5,882,864; 5,763,202; 5,747,264; 5,688,649; 5,552,277.

Nucleic Acids

The present invention relates to polynucleotides, such as DNAs, RNAs, and fragments thereof, which are expressed in prostate. These sequences are preferably selectively expressed in prostate, as compared to other tissues. SEQ ID NOS 1-58 and 67-72 show nucleotide sequences of polynucleotides in accordance with the present invention. By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in the prostate when compared to other tissue-types. In either case, a selectively expressed polynucleotide is a useful prostate marker and probe because its occurrence in a sample indicates the presence of prostate, having significant applications in diagnosis, therapy, histology, pathology, forensics, transplantation, and related areas.

SEQ ID NOS 1-58 and 67-72 show various nucleotide sequences for each selective polynucleotide of the present invention and corresponding polypeptide sequences. For each sequence, three different reading frames are displayed. The longest reading most typically represents an expressed polypeptide, but other reading frames are possible, so for completeness, the sequence list includes all possible open reading frames.

A selectively expressed polynucleotide is useful in a variety of different applications as described in greater details below. Because it is more abundant in prostate, it (or the polypeptide encoded by it) can be used as a diagnostic to test for the presence of prostate, e.g., in tissue sections, in a biopsy sample, in total RNA, etc. How to use polynucleotides in diagnostic assays is discussed below. In addition, the polynucleotides can serve as a target for therapy or drug discovery. A polypeptide, coded for by a selectively expressed polynucleotide, which is displayed on the cell-surface, can be a target for immunotherapy to treat, destroy, inhibit, etc., the diseased tissue. Selective transcripts can also be used in drug discovery schemes to identify pharmacological agents which suppress, inhibit, etc., their up-regulation, thereby preventing the phenotype associated with their expression. Thus, a selectively-expressed polynucleotide of the present invention has significant applications in diagnostic, therapeutic, prognostic, drug development, histology, pathology, and related areas.

A mammalian polynucleotide, or fragment thereof, of the present invention is a polynucleotide having a nucleotide sequence obtainable from a natural source. It therefore includes naturally-occurring normal, naturally-occurring mutant, and naturally-occurring polymorphic alleles (e.g., SNPs), differentially-spliced transcripts, splice-variants, etc. By the term “naturally-occurring,” it is meant that the polynucleotide is obtainable from a natural source, e.g., animal tissue and cells, body fluids, tissue culture cells, forensic samples. Natural sources include, e.g., living cells obtained from tissues and whole organisms, tumors, cultured cell lines, including primary and immortalized cell lines. Naturally-occurring mutations can include deletions (e.g., a truncated amino- or carboxy-terminus), substitutions, inversions, or additions of nucleotide sequence. These genes can be detected and isolated by polynucleotide hybridization according to methods which one skilled in the art would know, e.g., as discussed below.

A polynucleotide according to the present invention can be obtained from a variety of different sources. It can be obtained from DNA or RNA, such as polyadenylated mRNA or total RNA, e.g., isolated from tissues, cells, or whole organism. The polynucleotide can be obtained directly from DNA or RNA, from a cDNA library, from a genomic library, etc. The polynucleotide can be obtained from a cell or tissue (e.g., from an embryonic or adult tissues) at a particular stage of development, having a desired genotype, phenotype, disease status, etc.

The polynucleotides described in SEQ ID NOS 1-58 and 67-72 can be partial sequences that correspond to full-length, naturally-occurring transcripts. The present invention includes, as well, full-length polynucleotides that comprise these partial sequences, e.g., genomic DNAs and polynucleotides comprising a start and stop codon, a start codon and a polyA tail, a transcription start and a polyA tail, etc. These sequences can be obtained by any suitable method, e.g., using a partial sequence as a probe to select a full-length cDNA from a library containing full-length inserts. A polynucleotide which “codes without interruption” refers to a polynucleotide having a continuous open reading frame (“ORF”) as compared to an ORF which is interrupted by introns or other noncoding sequences.

Polynucleotides and polypeptides can be excluded as compositions from the present invention if, e.g., listed in a publicly available databases on the day this application was filed and/or disclosed in a patent application having an earlier filing or priority date than this application and/or conceived and/or reduced to practice earlier than a polynucleotide in this application.

As described herein, the phrase “an isolated polynucleotide which is SEQ ID NO,” or “an isolated polynucleotide which is selected from SEQ ID NO,” refers to an isolated nucleic acid molecule from which the recited sequence was derived (e.g., a cDNA derived from mRNA; cDNA derived from genomic DNA). Because of sequencing errors, typographical errors, etc., the actual naturally-occurring sequence may differ from a SEQ ID listed herein. Thus, the phrase indicates the specific molecule from which the sequence was derived, rather than a molecule having that exact recited nucleotide sequence, analogously to how a culture depository number refers to a specific cloned fragment in a cryotube.

As explained in more detail below, a polynucleotide sequence of the invention can contain the complete sequence as shown in SEQ ID NOS 1-58 and 67-72, degenerate sequences thereof, anti-sense, muteins thereof, genes comprising said sequences, full-length cDNAs comprising said sequences, complete genomic sequences, fragments thereof, homologs, primers, nucleic acid molecules which hybridize thereto, derivatives thereof, etc.

Genes of the present invention can be divided into different classes depending upon their function and/or structure, e.g., transmembrane (PR302, PR340, PR341, PR407, PR426, and PR498), cell signaling (PR293, PR325, and Pr407), translation-related (PR327), mediating protein/protein interactions (PR001, PR227, and PR449), RNA-binding (PR113-2 and PR155), transport and channel (PR051 and Pr219), nuclear/transcription (PR215, PR289, PR371, PR376, PR389, and PR471), oncogenesis-related (Pr057), and enzyme (PR133). This information can be used to determine how to use the genes and gene products. For instance, transmembrane and channel/transport proteins can be used as targets for antibodies for therapeutic and diagnostic applications. Polypeptides expressed intracellularly, e.g., signaling and nuclear/transcription polypeptides can be targets to disrupt cell function and initiate apoptosis. In a therapeutic or diagnostic application, it may be desired to pick multiple transmembrane targets, or genes from each of the different classes, to enhance therapeutic and labeling utilities.

Pr001

Pr001 (also known as Pra001) (related to NM_{—}02404 and AK02556) is a TPR domain polypeptide containing 665 amino acids. The nucleotide and amino acid sequences of Pr001 are shown in SEQ ID NOS 1 and 2. It contains a coiled-coil at amino acid positions 5-48, and six TPR domains at amino acid positions 133-166, 167-200, 201-234, 282-315, 316-349, and 350-383. TPR motifs are 34 amino acid repeats found in a variety of proteins with diverse functions. They play a role in protein-protein interactions, providing a scaffolding for assembling protein complexes involved in different cellular functions, including, but not limited to, cell signaling, transcription, and protein targeting. See, e.g., Owens-Grillo et al., J. Biol. Chem., 271:13,468-13,475, 1996.

All or part of Pr001 is located in genomic DNA represented by AC00424 1, and BAC clone RPCI3-197B 17. It is distributed in at least 16 separate exons. The present invention relates to any isolated introns and exons that are present in such clone. Such introns and exons can be routinely determined.

As indicated by its expression profile, Pr001 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr001 aberrations include, but are not limited to, persistent Mullerian duct syndrome and nocturnal enuresis.

Disorders associated with Pr001 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr001 expression can occur in cell types other than prostate, and thus can have a function outside of its role in prostate.

Nucleic acids of the present invention map to chromosomal band 12q13-13.1. There are a number of different disorders which have been mapped to, or in close proximity to, this chromosome location. These include, e.g., Glioma, Monilethrix, Liver disease susceptibility to from hepatotoxins or viruses, white sponge nevus, Pachyonychia congenita Jadassohn-Lewandowsky type, Myopathy congenital, Meesmann corneal dystrophy, Keratoderma palmoplantar nonepidermolytic, Epidermolytic hyperkeratosis, Epidermolysis bullosa simplex, Koebner Dowling-Meara and Weber-Cockayne, Diabetes insipidus, nephrogenic autosomal recessive, Achalasia-addisonianism-alacrimia syndrome, Ichthyosis bullosa of Siemens, Palmoplantar keratoderma Bothnia type, Myxoid liposarcoma, Achondrogenesis-hypochondrogenesis type II, Wagner syndrome type II, Stickler syndrome type I, SMED Strudwick type, SED congenita, and Osteoarthrosis precocious. Nucleic acids of the present invention can be used as linkage markers, diagnostic targets, therapeutic targets, for any of the mentioned disorders, as well as any disorders or genes mapping in proximity to it.

Pr051a

Pr051a codes for an ion transport polypeptide containing 1039 amino acids. The nucleotide and amino acid sequences of Pr051a are shown in SEQ ID NOS 3 and 4. It contains 7 transmembrane domains at about amino acid positions 643-662, 674-707, 747-769, 776-798, 813-835, 848-865, and 955-974. An ion transport domain is present at about amino acids 717-929. It shares sequence identity with a calcium channel protein.

All or part of Pr051a is located in genomic DNA represented by AC005538, BAC-ID: RP11-332L11, and Contig NT_{—}005414. The present invention relates to any isolated introns and exons that are present in such clone. Such introns and exons can be routinely determined.

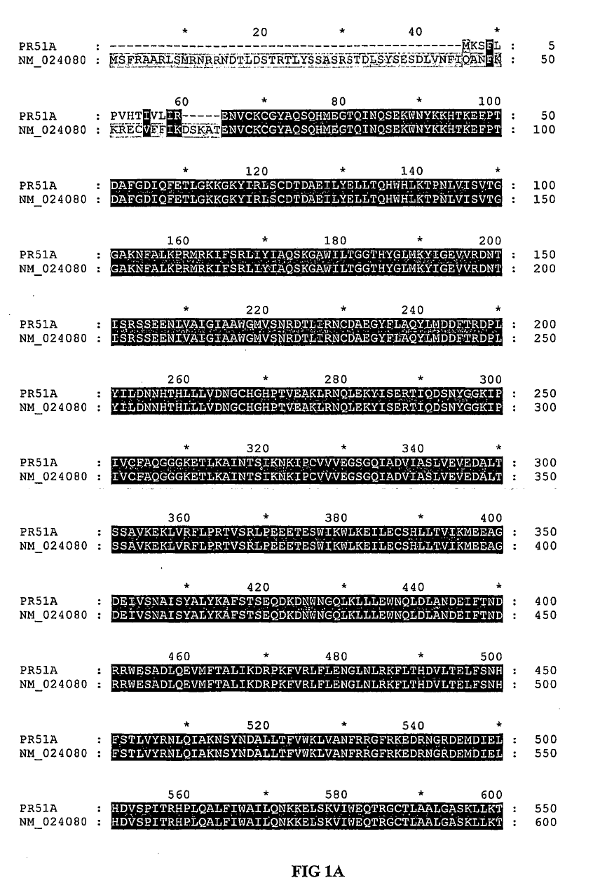

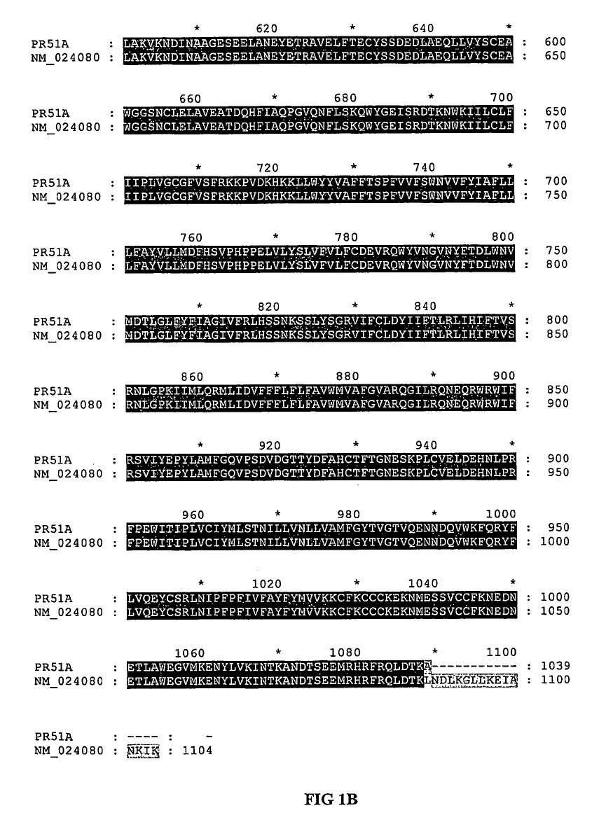

The polypeptide coded for by Pr051a exhibits sequence identity to human NM_{—}024080. Alignment with it is shown in FIG. 1. Regions of variation, overlap, or non-overlap can be separately claimed.

As indicated by its expression profile, Pr051a has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors.

Disorders associated with Pr051a can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr051a expression can occur in cell types other than prostate, and thus can have a function outside of its role in prostate.

Nucleic acids of the present invention map to chromosomal band 2q37.1-q37.2. There are a number of different disorders which have been mapped to, or in close proximity to, this chromosome location. These include, e.g., Systemic Lupus Erythematosus, Susceptibility to, 2 (SLEB2), Brachydactyl-Mental Retardation Syndrome, and Finnish Lethal Neonatal Metabolic Syndrome. Nucleic acids of the present invention can be used as linkage markers, diagnostic targets, therapeutic targets, for any of the mentioned disorders, as well as any disorders or genes mapping in proximity to it.

Pr057

Pr057 is 400 amino acid polypeptide. It is related to the TFG gene (NM_{—}006070). See, e.g., Mencinger et al., Genomics, 41:327-331, 1997. Mouse homologs are coded for by NM_{—}019678. The nucleotide and amino acid sequences of Pr057 are shown in SEQ ID NOS 5 and 6. It has an octicosapeptide repeat at about amino acid positions 53-82, a coiled coil at about 98-124, and a proline-rich region at about 295-397. It is involved in oncogenesis.

All or part of Pr057 is located in genomic DNA represented by AC021873, NT_{—}005863, and BAC clone RP11-475J9. The present invention relates to any isolated introns and exons that are present in such clone. Such introns and exons can be routinely determined.

As indicated by its expression profile, Pr057 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr057 aberrations include, but is not limited to, prostate cancer.

Disorders associated with Pr057 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr057 expression can occur in cell types other than prostate, and thus can have a function outside of its role in prostate.

Nucleic acids of the present invention map to chromosomal 3q11-q12. There are a number of different disorders which have been mapped to, or in close proximity to, this chromosome location. These include, e.g., thyroid carcinoma, dementia, protein S deficiency, coproporphyria, and harderoporphyrinuria. Nucleic acids of the present invention can be used as linkage markers, diagnostic targets, therapeutic targets, for any of the mentioned disorders, as well as any disorders or genes mapping in proximity to it.

Pr104

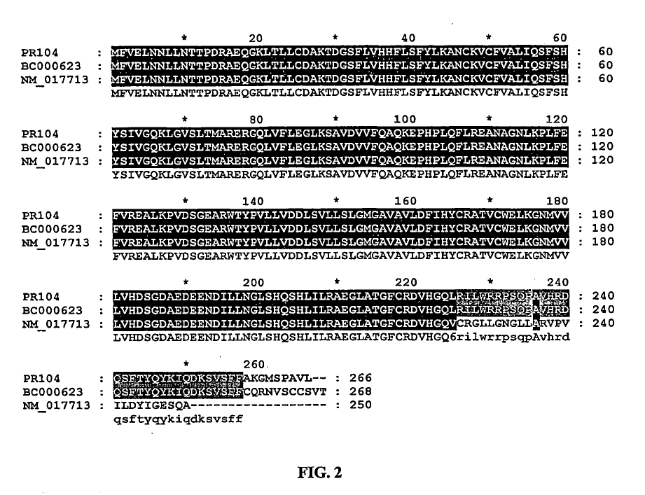

Pr104 (also known as Pra104) (related to BC000623 and NM_{—}017713) codes for a prostate-selective polypeptide containing 266-amino acids. The nucleotide and amino acid sequences of Pr104 are shown in SEQ ID NOS 7 and 8. A mouse homolog is AK011270. FIG. 2 shows its alignment with BC000623 and NM_{—}017713. Regions of overlap, variation, and non-overlap can be separately claimed. Polymorphisms are listed in Table 1.

All or part of Pr104 is located in genomic DNA represented by AC023230, NT_{—}005991, and BAC clone RP11-708J19. The gene contains at least seven exons. The present invention relates to any isolated introns and exons that are present in such clone. Such introns and exons can be routinely determined.

As indicated by its expression profile, Pr104 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr104 aberrations include, but is not limited to, prostatic adenocarcinoma. See, e.g., Dahiya et al., Int. J. Cancer, 71(1):20-5, 1997.

Disorders associated with Pr104 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr104 expression can occur in cell types other than prostate, and thus can have a function outside of its role in prostate. Nucleic acids of the present invention map to chromosomal band 3p24.3. There are a number of different disorders which have been mapped to, or in close proximity to, this chromosome location, e.g., thyroid hormone resistance. Nucleic acids of the present invention can be used as linkage markers, diagnostic targets, therapeutic targets, for any of the mentioned disorders, as well as any disorders or genes mapping in proximity to it.

Pr113-2

Pr113-2 (related to NM_{—}016648, AK000274, and Hs.278635) codes for a polypeptide containing 497 amino acids. The nucleotide and amino acid sequences of Pr113-2 are shown in SEQ ID NOS 9 and 10. It has a RRM domain at amino acid positions 60-136 and a coiled coil domain at amino acid positions 154-177. The coiled coil domain is involved in stabilizing protein-protein interactions. It is one of the principal oligomerization motifs in polypeptides. See, e.g. Burkhard et al., Trends Cell Biol., 11(2):82-8, 2001. The RNA recognition motif (RRM) is an 80 amino acid consensus sequence that is conserved in many RNA binding proteins and is involved in the recognition and binding to RNA. These are commonly found in ribonucleoproteins (RNPs).

All or part of Pr113-2 is located in genomic DNA represented by AC005073 and BAC clone RP11-79506. The present invention relates to any isolated introns and exons that are present in such clone. Such introns and exons can be routinely determined.

As indicated by its expression profile, Pr113-2 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors.

Disorders associated with Pr113-2 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr113-2 expression can occur in cell types other than prostate, and thus can have a function outside of its role in prostate.

Pr133

Pr133 (related to NM_{—}007024) codes for a decarboxylase containing 351 amino acids. The nucleotide and amino acid sequences of Pr133 are shown in SEQ ID NOS 11 and 12. A mouse homolog is represented by NM_{—}019704. It contains an orotidine 5′-phosphate decarboxylase domain at about amino acid positions 274-298, a bipartite nuclear localization signal domain at about amino acid positions 282-299, six transmembrane domains at about amino acid positions 13-35, 98-120, 127-149, 164-183, 190-208, and 223-245, and a signal peptide at about amino acid positions 1-40.

All or part of Pr133 is located in genomic DNA represented by AC002481, NT_{—}006014, and BAC clone LUCA12. The present invention relates to any isolated introns and exons that are present in such clone. Such introns and exons can be routinely determined.

As indicated by its expression profile, Pr133 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr133 aberrations include, but are not limited to, prostate cancer and prostatic adenocarcinoma.

Disorders associated with Pr133 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr133 expression can occur in cell types other than prostate, and thus can have a function outside of its role in prostate.

Nucleic acids of the present invention map to chromosomal band 3p21.3. There are a number of different disorders which have been mapped to, or in close proximity to, this chromosome location. These include, e.g., epidermolysis bullosa dystrophica, turcot syndrome with glioblastoma, colorectal cancer, Muir-Torre family cancer syndrome, and epidermolysis bullosa dystrophica. Nucleic acids of the present invention can be used as linkage markers, diagnostic targets, therapeutic targets, for any of the mentioned disorders, as well as any disorders or genes mapping in proximity to it.

Pr155

Pr155 codes for a polypeptide containing 230 amino acids. The nucleotide and amino acid sequences of Pr155 are shown in SEQ ID NOS 13 and 14.

All or part of Pr155 is located in genomic DNA represented by AL353587 and BAC clone RP13-178D16. The gene contains at least eight separate exons. The present invention relates to any isolated introns and exons that are present in such clone. Such introns and exons can be routinely determined.

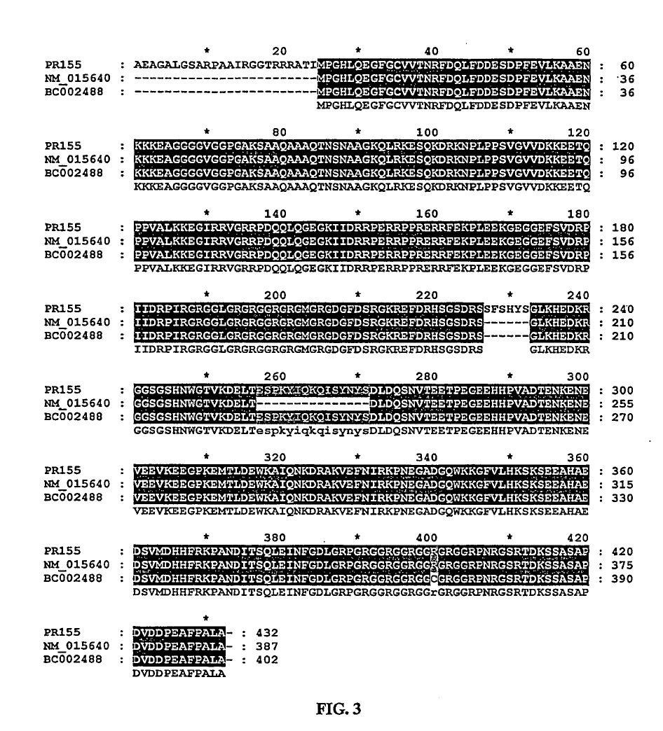

Pr155 is related to PAI-1 mRNA-binding protein (NM_{—}015640 and BC002488). Regions of overlap, variation, and non-overlap can be separately claimed. Alignments are shown in FIG. 3.

As indicated by its expression profile, Pr155 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr155 aberrations includes, but is not limited to, prostate cancer.

Disorders associated with Pr155 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr155 expression can occur in cell types other than prostate, and thus can have a function outside of its role in prostate.

Nucleic acids of the present invention map to chromosomal band Xp 11.1. There are a number of different disorders which have been mapped to, or in close proximity to, this chromosome location. These include, e.g., androgen insensitivity, spinal and bulbar muscular atrophy of Kennedy, breast cancer male with Reifenstein syndrome, perineal hypospadias, and insulin-dependent Diabetes Mellitis (IDDMX). Nucleic acids of the present invention can be used as linkage markers, diagnostic targets, therapeutic targets, for any of the mentioned disorders, as well as any disorders or genes mapping in proximity to it.

Pr215

Pr215 codes for a regulatory protein containing 894 amino acids. The nucleotide and amino acid sequences of Pr215 are shown in SEQ ID NOS 15 and 16. Partial sequences of it, e.g., Hs.29299 and Hs.12699, can be excluded from the present invention. Pr215 comprises four MBT domains at about amino acid positions 44-144, 152-256, 266-372, and 380-477; a SAM domain at about amino acid positions 821-887; SAM/Pointed domain at about amino acid positions 809-888; and a bipartite nuclear localization signal domain at about amino acid positions 690-707. The presence of MBT (e.g., Usui et al., Gene, 248:127-135, 2000) and SAM (e.g., Kyba and Brock, Developmental Genetics, 22:74-84, 1998) domains indicate a function in transcription.

All or part of Pr215 is located in genomic DNA represented by AL357147, NT_{—}025820, and BAC clone RP1-29M21. The gene for it contains at least 17 separate exons. The present invention relates to any isolated introns and exons that are present in such clone. Such introns and exons can be routinely determined.

As indicated by its expression profile, Pr215 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr215 aberrations include, but are not limited to, prostate cancer and prostatic adenocarcinoma.

Disorders associated with Pr215 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr215 expression can occur in cell types other than prostate, and thus can have a function outside of its role in prostate.

Nucleic acids of the present invention map to chromosomal band 10q11.2. There are a number of different disorders which have been mapped to, or in close proximity to, this chromosome location. These include, e.g., Usher syndrome type 1D, Insulin-dependent diabetes mellitus-10, Cockayne syndrome-2 late onset, Thyroid papillary carcinoma, Multiple endocrine neoplasia, Medullary thyroid carcinoma, Hirschsprung disease, and Thyroid papillary carcinoma. Nucleic acids of the present invention can be used as linkage markers, diagnostic targets, therapeutic targets, for any of the mentioned disorders, as well as any disorders or genes mapping in proximity to it.

Pr219

Pr219 (related to AK021629) codes for a 200 amino acid ATPase. The nucleotide and amino acid sequences of Pr219 are shown in SEQ ID NOS 17 and 18. It has a V-type ATPase domain (e.g., involved in proton pumping) at about amino acid positions 59-175.

All or part of Pr219 is located in genomic DNA represented by NT_{—}022618. it contains at least 2 exons. The present invention relates to any isolated introns and exons that are present in such clone. Such introns and exons can be routinely determined.

As indicated by its expression profile, Pr219 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr219 aberrations include, but are not limited to, cancer and metastasis.

Disorders associated with Pr219 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr219 expression can occur in cell types other than prostate, and thus can have a function outside of its role in prostate. Nucleic acids of the present invention map to chromosome 3.

Pr227

Pr227 (related to AL080094) codes for a 264 amino acid protein-binding polypeptide. The nucleotide and amino acid sequences of Pr227 are shown in SEQ ID NOS 19 and 20. It contains a thrombospondin type 1 domain at about amino acid positions 74-139 and a somatomedin B domain at about amino acid positions 50-70.

All or part of Pr227 is located in genomic DNA represented by AC022893 and BAC clone RP11-531A24. The gene contains at least five exons. The present invention relates to any isolated introns and exons that are present in such clone. Such introns and exons can be routinely determined.

As indicated by its expression profile, Pr227 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr227 aberrations includes, but is not limited to, prostate cancer.

Disorders associated with Pr227 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr227 expression can occur in cell types other than prostate, and thus can have a function outside of its role in prostate.

Nucleic acids of the present invention map to chromosomal band 8q21.1. There are a number of different disorders which have been mapped to, or in close proximity to, this chromosome location. These include, e.g., familial febrile convulsions, Charcot-Marie-Tooth neuropathy, Adrenal hyperplasia, congenital due to 11-beta-hydroxylase deficiency, Aldosteronism, Hypoaldosteronism, Nijmegen breakage syndrome, Low renin hypertension susceptability, Refsum disease, DECR deficiency, Giant cell hepatitis, Colon adenocarcinoma, and Achromatopsia-3. Nucleic acids of the present invention can be used as linkage markers, diagnostic targets, therapeutic targets, for any of the mentioned disorders, as well as any disorders or genes mapping in proximity to it.

Pr289

Pr289 (related to NM_{—}015484) codes for a regulatory protein containing 243 amino acids. It is also known as GCIP-interacting protein P29. The nucleotide and amino acid sequences of Pr289 are shown in SEQ ID NOS 21 and 22. It comprises bipartite nuclear localization signal at about amino acids 48-65 and 71-88. A rodent homolog is AK003787.

All or part of Pr289 is located in genomic DNA represented by AL031432. NT_{—}004391, and BAC clone RP3-465N24. It has about seven exons. The present invention relates to any isolated introns and exons that are present in such clone. Such introns and exons can be routinely determined.

As indicated by its expression profile, Pr289 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr289 aberrations includes, but is not limited to, prostate cancer.

Disorders associated with Pr289 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr289 expression can occur in cell types other than prostate, and thus can have a function outside of its role in prostate.

Nucleic acids of the present invention map to chromosomal band 1p36.13-p35.1. There are a number of different disorders which have been mapped to, or in close proximity to, this chromosome location. These include, e.g., colorectal cancer resistance to, glucose transport defect blood-brain barrier, Kostmann neutropenia, myopathy due to succinate dehydrogenase deficiency, Charcot-Marie-Tooth neuropathy-2A, galactose epimerase deficiency, muscular dystrophy congenital with early spine rigidity, galactose epimerase deficiency, corneal dystrophy crystalline Schnyder, malignant melanoma cutaneous, breast cancer ductal. Bartter syndrome type 3, neuroblastoma, cataracts, hypophosphatasia, Schwartz-Jampel syndrome, myopathy due to succinate dehydrogenase deficiency, glaucoma, rhabdomyosarcoma alveolar, enolase deficiency. Nucleic acids of the present invention can be used as linkage markers, diagnostic targets, therapeutic targets, for any of the mentioned disorders, as well as any disorders or genes mapping in proximity to it.

Pr293

Pr293 (related to NM_{—}002865) is a member of the Ras family containing 212 amino acids. The rodent homolog is NM_{—}021518. The nucleotide and amino acid sequences of Pr293 are shown in SEQ ID NOS 23 and 24. It contains an ATP/GTP-binding site motif A (P-loop) at about amino acid positions 13-20, a sigma-54 factor interaction protein domain at about amino acid positions 9-22, and a Ras GTPase domain at about amino acids 8-212. It has a role in cell signalling.

All or part of Pr293 is located in genomic DNA represented by AC068389, NT_{—}008012, and BAC clone RP11-91I20. The present invention relates to any isolated introns and exons that are present in such clone. Such introns and exons can be routinely determined.

As indicated by its expression profile, Pr293 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr293 aberrations includes, but is not limited to, prostate cancer.

Disorders associated with Pr293 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr293 expression can occur in cell types other than prostate, and thus can have a function outside of its role in prostate.

Nucleic acids of the present invention map to chromosomal band 8q12. There are a number of different disorders which have been mapped to, or in close proximity to, this chromosome location. These include, e.g., salivary gland adenoma. Nucleic acids of the present invention can be used as linkage markers, diagnostic targets, therapeutic targets, for any of the mentioned disorders, as well as any disorders or genes mapping in proximity to it.

Pr302

Pr302 codes for a polypeptide containing 410 amino acids. The nucleotide and amino acid sequences of Pr302 are shown in SEQ ID NOS 25 and 26. It contains transmembrane domains at about amino acid positions 350-369 and 379-401, and an incomplete ERM domain at about amino acid positions 81-187. The presence of the ERM domain indicates its role in linking the cytoskeleton to cell membranes. A partial mouse homolog of human Pr302 is AK017174. Polymorphisms are shown in Table 1.

All or part of Pr302 is located in genomic DNA represented by NT_{—}009935. The gene contains at least 12 separate exons. The present invention relates to any isolated introns and exons that are present in such clone. Such introns and exons can be routinely determined.

As indicated by its expression profile, Pr302 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr302 aberrations include, but are not limited to prostate cancer and prostate adenocarcinoma.

Disorders associated with Pr302 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr302 expression can occur in cell types other than prostate, and thus can have a function outside of its role in prostate.

Nucleic acids of the present invention map to chromosomal band 13q14. There are a number of different disorders which have been mapped to, or in close proximity to, this chromosome location. These include, e.g., Leukemia, Rieger syndrome, Retinoblastoma, Pinealoma with bilateral retinoblastoma, Bladder cancer, Rhabdomyosarcoma alveolar, and Osteosarcoma. Nucleic acids of the present invention can be used as linkage markers, diagnostic targets, therapeutic targets, for any of the mentioned disorders, as well as any disorders or genes mapping in proximity to it.

Pr324

Pr324 (related to NM_{—}006674) codes for a polypeptide containing 132 amino acids. It is present in multiple copies within the human MHC class I region. The nucleotide and amino acid sequences of Pr324 are shown in SEQ 27 and 28.

All or part of Pr324 is located in genomic DNA represented by AC018433, NT_{—}007592, and BAC clone RP11-23C1. The present invention relates to any isolated introns and exons that are present in such clone. Such introns and exons can be routinely determined.

As indicated by its expression profile, Pr324 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr324 aberrations include, but are not limited to, prostate cancer

Disorders associated with Pr324 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr324 expression can occur in cell types other than prostate, and thus can have a function outside of its role in prostate.

Nucleic acids of the present invention map to chromosomal band 6p21.3. There are a number of different disorders which have been mapped to, or in close proximity to, this chromosome location. These include, e.g., Stickler syndrome type II, Ragweed sensitivity, Retinitis pigmentosa-14, Paget disease of bone, Sialidosis type I, Sialidosis type II, Renal glucosuria, Laryngeal adductor paralysis, Beryllium disease chronic susceptibility to, Psoriasis susceptibility-1, Atrial septal defect secundum type, Ossification of posterior longitudinal ligament of spine, Pemphigoid susceptibility to, Hemochromatosis, Ehlers-Danlos-like syndrome, Dyslexia specific, Diabetes mellitus insulin-dependent-1, Deafness autosomal dominant 13, C4 deficiency, Bare lymphocyte syndrome type I due to TAP2 deficiency, Ankylosing spondylitis, Adrenal hyperplasia congenital due to 21-hydroxylase deficiency, C2 deficiency, and OSMED syndrome. Nucleic acids of the present invention can be used as linkage markers, diagnostic targets, therapeutic targets, for any of the mentioned disorders, as well as any disorders or genes mapping in proximity to it.

Pr325

Pr325 (related to NM_{—}006835) codes for cyclin-1, a member of the cyclin family of CDK kinases, and contains 377 amino acids. The nucleotide and amino acid sequences of Pr325 are shown in SEQ ID NOS 29 and 30. A rodent homolog is NM_{—}017367.

All or part of Pr325 is located in genomic DNA represented by AC079051, NT_{—}006068, BAC clone RP11-136P18. The present invention relates to any isolated introns and exons that are present in such clone. Such introns and exons can be routinely determined.

As indicated by its expression profile, Pr325 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr325 aberrations include, but is not limited to prostate cancer, e.g., where the progression of the prostate cancer is associated with the loss of the gene.

Disorders associated with Pr325 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr325 expression can occur in cell types other than prostate, and thus can have a function outside of its role in prostate.

Nucleic acids of the present invention map to chromosomal band 4q16-q20. Nucleic acids of the present invention can be used as linkage markers, diagnostic targets, therapeutic targets, for any disorders or genes mapping in proximity to it.

Pr327

Pr327 (related to NM_{—}001568 and Hs.106673) codes for a polypeptide containing 445 amino acids. It is also known as EIF3S6, eukaryotic translation initiation factor 3, subunit 6 (48 kD). A rodent homolog is AK002576. The nucleotide and amino acid sequences of Pr327 are shown in SEQ ID NOS 31 and 32,

All or part of Pr327 is located in genomic DNA represented by AP001331, NT_{—}008144, and BAC clone KB1153C10. The gene contains at least 13 exons. The present invention relates to any isolated introns and exons that are present in such clone. Such introns and exons can be routinely determined.

As indicated by its expression profile, Pr327 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr327 aberrations include, but is not limited to prostate cancer

Disorders associated with Pr327 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr327 expression can occur in cell types other than prostate, and thus can have a function outside of its role in prostate.

Nucleic acids of the present invention map to chromosomal band 8q23. There are a number of different disorders which have been mapped to, or in close proximity to, this chromosome location. These include, e.g., Cohen syndrome. Nucleic acids of the present invention can be used as linkage markers, diagnostic targets, therapeutic targets, for any of the mentioned disorders, as well as any disorders or genes mapping in proximity to it.

Pr329

Pr329 (related to NM_{—}003953) codes for a polypeptide containing 269 amino acids. It is also known as myelin protein-zero (and is associated with types of Charcot-Marie-Tooth disease). The nucleotide and amino acid sequences of Pr329 are shown in SEQ ID NOS 33 and 34.

All or part of Pr329 is located in genomic DNA represented by AC073241 and NT_{—}004668, and Bac clone RP11-392B20. The gene contains at least six exons. The present invention relates to any isolated introns and exons that are present in such clone. Such introns and exons can be routinely determined.

As indicated by its expression profile, Pr329 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors.

Disorders associated with Pr329 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr329 expression can occur in cell types other than prostate, and thus can have a function outside of its role in prostate.

Nucleic acids of the present invention map to chromosomal band 1q22. There are a number of different disorders which have been mapped to, or in close proximity to, this chromosome location. These include, e.g., Nemaline myopathy-1, Porphyria variegata, Hypomyelination congenital, Charcot-Marie-Tooth neuropathy-1B, ivax malaria susceptibility, Insensitivity to pain, Dejerine-Sottas disease myelin P(0), and CD zeta chain deficiency. Nucleic acids of the present invention can be used as linkage markers, diagnostic targets, therapeutic targets, for any of the mentioned disorders, as well as any disorders or genes mapping in proximity to it.

Pr340

Pr340 (related to NM_{—}032016) codes for a transmembrane polypeptide containing 234 amino acids. The nucleotide and amino acid sequences of Pr340 are shown in SEQ ID NOS 35 and 36. It contains four transmembrane domains at about amino acid positions 55-74, 94-116, 123-142, and 152-171.

All or part of Pr340 is located in genomic DNA represented by AC006033, NT_{—}007819, and BAC clone RP11-121AB. It contains at least 8 exons. The present invention relates to any isolated introns and exons that are present in such clone. Such introns and exons can be routinely determined.

As indicated by its expression profile, Pr340 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr340 aberrations includes, but is not limited to, prostatic adenocarcinoma.

Disorders associated with Pr340 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr340 expression can occur in cell types other than prostate, and thus can have a function outside of its role in prostate.

Nucleic acids of the present invention map to chromosomal band 7p14. There are a number of different disorders which have been mapped to, or in close proximity to, this chromosome location. These include, e.g., Neuronal type D Charcot-Maine-Tooth Disease, Growth hormone deficient dwarfism, Colton blood group, and Alpha-ketoglutarate dehydrogenase deficiency. Nucleic acids of the present invention can be used as linkage markers, diagnostic targets, therapeutic targets, for any of the mentioned disorders, as well as any disorders or genes mapping in proximity to it.

Pr341

Pr341 (related to AB007916 and Hs.214646) codes for transmembrane polypeptide having 391 amino acids. The nucleotide and amino acid sequences of Pr341 are shown in SEQ ID NOS 37 and 38. Three transmembrane domains are located at about amino acid positions 75-97, 107-129, and 142-164.

All or part of Pr341 is located in genomic DNA represented by AL031282 and NT_{—}026214. The gene contains at least six separate exons. The present invention relates to any isolated introns and exons that are present in such clone. Such introns and exons can be routinely determined

As indicated by its expression profile, Pr341 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr341 aberrations include, but is not limited to, prostate cancer.

Disorders associated with Pr341 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr341 expression can occur in cell types other than prostate, and thus can have a function outside of its role in prostate.

Nucleic acids of the present invention map to chromosomal band 1p36.21-p36.33. There are a number of different disorders which have been mapped to, or in close proximity to, this chromosome location. These include, e.g., Elliptocytosis-1, Erythrokeratodermia variabilis, Glaucoma 3 primary infantile, Rhabdomyosarcoma alveolar, Ehlers-Danlos syndrome type VI, C1q deficiency, Homocystinuria due to MTHFR deficiency, Neuroblastoma, and brain cancer. Nucleic acids of the present invention can be used as linkage markers, diagnostic targets, therapeutic targets, for any of the mentioned disorders, as well as any disorders or genes mapping in proximity to it.

Pr371

Pr371 codes for a 110-amino acid apurinic/apyrimidinic (AP) endonuclease. The nucleotide and amino acid sequences of Pr371 are shown in SEQ ID NOS 39 and 40. Polypmorphisms are shown in Table 1.

All or part of Pr371 is located in genomic DNA represented by AC076961 and BAC clone RP11-392H1. The gene has at least three exons. The present invention relates to any isolated introns and exons that are present in such clone. Such introns and exons can be routinely determined.

As indicated by its expression profile, Pr371 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors.

Disorders associated with Pr371 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr371 expression can occur in cell types other than prostate, and thus can have a function outside of its role in prostate.

Nucleic acids of the present invention map to chromosomal band 3q24. There are a number of different disorders which have been mapped to, or in close proximity to, this chromosome location. These include, e.g., glaucoma, Hailey-Hailey disease, Hemosiderosis systemic due to aceruloplasminemia, Hypocalcemia, Neonatal hyperparathyroidism, Night blindness congenital stationery rhodopsin-related, Retinitis pigmentosa, and Hypoceruloplasmninemia. Nucleic acids of the present invention can be used as linkage markers, diagnostic targets, therapeutic targets, for any of the mentioned disorders, as well as any disorders or genes mapping in proximity to it.

Activity of Pr371, and biologically active fragments thereof, can be determined routinely using conventional assay methods. Unk et al., J. Biol. Chem., Vol. 275, Issue 29, 22427-22434, Jul. 21, 2000. An AP endonuclease can be used to remove DNA which contains an abasic site, and then the site can be repaired with DNA polymerase and DNA ligase.

Pr376

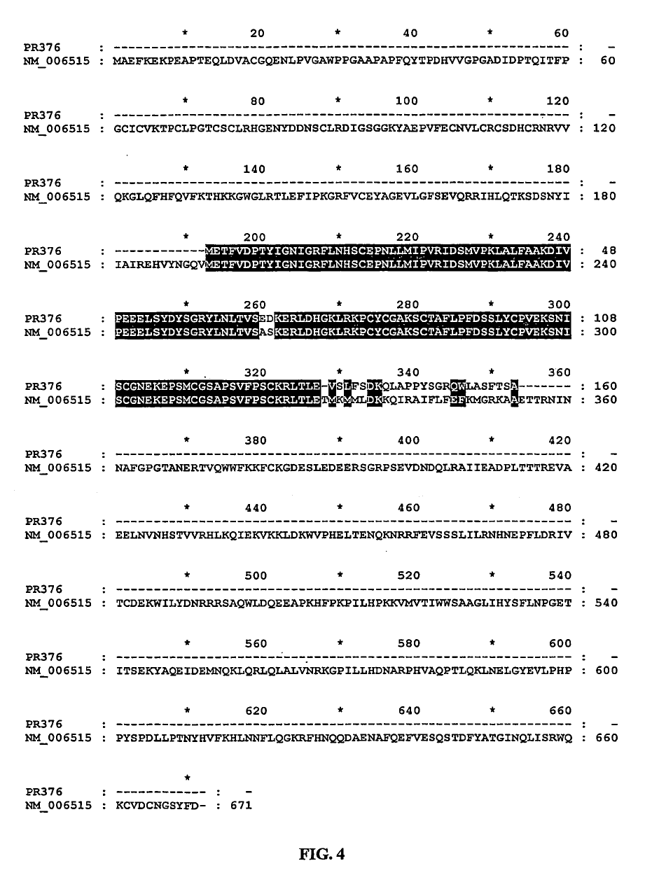

Pr376 (related to NM_{—}006515 and Hs.265855) codes for a 160 amino acid regulatory factor. The nucleotide and amino acid sequences of Pr376 are shown in SEQ ID NOS 41 and 42. It contains a SET-domain (found in transcriptional) at about amino acid positions 1-62 and a post-SET domain at about amino acid positions 78-94. Alignment with NM_{—}006515 is shon in FIG. 4. Regions of variation, overlap, and non-overlap can be separately claimed.

All or part of Pr376 is located in genomic DNA represented by AC023483, NT_{—}005927, and BAC clone RP11-453A3. It contains at least two exons. The present invention relates to any isolated introns and exons that are present in such clone. Such introns and exons can be routinely determined.

As indicated by its expression profile, Pr376 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr376 aberrations include, but is not limited to prostate cancer.

Disorders associated with Pr376 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr376 expression can occur in cell types other than prostate, and thus can have a function outside of its role in prostate.

Nucleic acids of the present invention map to chromosomal band 3p24.2-p26. There are a number of different disorders which have been mapped to, or in close proximity to, this chromosome location. These include, e.g., Long QT syndrome-3, Marfan syndrome, Thyroid hormone resistance, von Hippel-Lindau syndrome, Muscular dystrophy limb-girdle type, Pancreatic endocrine tumors, Xeroderma pigmentosum, Cardiomyopathy dilated, Renal cell carcinoma, Biotinidase deficiency, Fanconi anemia, and Renal cell carcinoma. Nucleic acids of the present invention can be used as linkage markers, diagnostic targets, therapeutic targets, for any of the mentioned disorders, as well as any disorders or genes mapping in proximity to it.

Pr389

Pr389 (related to NM_{—}005870) codes for a 153 amino acid polypeptide. It is also known as SAP18, Sin3-associated polypeptide, a protein involved in transcriptional regulation. The nucleotide and amino acid sequences of Pr389 are shown in SEQ ID NOS 43 and 44. A rodent homolog is NM_{—}009119.

All or part of Pr389 is located in genomic DNA represented by AL512652, NT_{—}009917, and BAC clone RP11-261p13. The present invention relates to any isolated introns and exons that are present in such clone. Such introns and exons can be routinely determined

As indicated by its expression profile, Pr389 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr389 aberrations includes, but is not limited to prostate cancer, including the progression of prostate cancer.

Disorders associated with Pr389 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr389 expression can occur in cell types other than prostate, and thus can have a function outside of its role in prostate.

Nucleic acids of the present invention map to chromosomal band 13cen-13q14.2. There are a number of different disorders which have been mapped to, or in close proximity to, this chromosome location. These include, e.g., Stem-cell leukemia/lymphoma syndrome, Deafness autosomal recessive, Cataract zonular pulverulent-2, Ectodermal dysplasia hidrotic, Muscular dystrophy limb-girdle type 2C, Pancreatic agenesis, Moebius syndrome, Breast cancer 2 early onset, Enuresis nocturnal, Leukemia chronic lymphocytic B-cell, Rieger syndrome type 2, Retinoblastoma, Pinealoma with bilateral retinoblastoma, Bladder cancer, Rhabdomyosarcoma alveolar, and Osteosarcoma. Nucleic acids of the present invention can be used as linkage markers, diagnostic targets, therapeutic targets, for any of the mentioned disorders, as well as any disorders or genes mapping in proximity to it.

Pr398

Pr398 (related to partial clone Hs.74655) codes for a polypeptide containing 190 amino acids. The nucleotide and amino acid sequences of Pr398 are shown in SEQ ID NOS 45 and 46. Table 1 lists polymorphisms.

All or part of Pr398 is located in genomic DNA represented by AC005837, NT_{—}010641, and BAC clone hRPK.318_A_{—}15. The present invention relates to any isolated introns and exons that are present in such clone. Such introns and exons can be routinely determined.

As indicated by its expression profile, Pr398 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr398 aberrations includes, but is not limited to prostate cancer.

Disorders associated with Pr398 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr398 expression can occur in cell types other than prostate, and thus can have a function outside of its role in prostate.

Nucleic acids of the present invention map to chromosomal band 17q25.1. There are a number of different disorders which have been mapped to, or in close proximity to, this chromosome location. These include, e.g., Neuralgic amyotrophy with predilection for brachial plexus, Adrenoleukodystrophy pseudoneonatal, Diabetes mellitus type II, Russell-Silver syndrome, and Campomelic dysplasia with autosomal sex reversal. Nucleic acids of the present invention can be used as linkage markers, diagnostic targets, therapeutic targets, for any of the mentioned disorders, as well as any disorders or genes mapping in proximity to it.

Pr407

Pr407 (related to NM_{—}017830) codes for a 245 amino acid transmembrane protein. It is also known as ovarian carcinoma immunoreactive antigen (OCIA). The nucleotide and amino acid sequences of Pr407 are shown in SEQ ID NOS 47 and 48. It contains a transmembrane domain as about amino acid positions 7-92, and an ITAM domain at about amino acid positions 16-217. Pr407 is a transmembrane signaling protein. A mouse homolog is NM_{—}023429.

All or part of Pr407 is located in genomic DNA represented by A079927, NT_{—}016632, and BAC clone RP11-702A23. The gene contains at least nine different exons. The present invention relates to any isolated introns and exons that are present in such clone. Such introns and exons can be routinely determined.

As indicated by its expression profile, Pr407 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors.

Disorders associated with Pr407 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr407 expression can occur in cell types other than prostate, and thus can have a function outside of its role in prostate.

Nucleic acids of the present invention map to chromosomal band 4p11-p12. There are a number of different disorders which have been mapped to, or in close proximity to, this chromosome location. These include, e.g., ovarian cancer. Nucleic acids of the present invention can be used as linkage markers, diagnostic targets, therapeutic targets, for any of the mentioned disorders, as well as any disorders or genes mapping in proximity to it.

Pr413

Pr413 (related to Hs.44468 and AL50197) codes for a polypeptide containing 274 amino acids. The nucleotide and amino acid sequences of Pr413 are shown in SEQ ID NOS 49 and 50. A mouse homolog is AK003287.

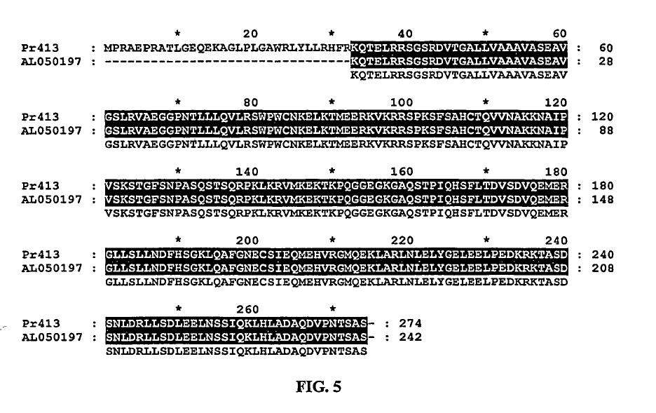

All or part of Pr413 is located in genomic DNA represented by AL121834, NT_{—}025741, and BAC clone RP3-509113. Pr413 contains at least five exons. The present invention relates to any isolated introns and exons that are present in such clone. Such introns and exons can be routinely determined. Alignment with AL50197 is shown in FIG. 5, indicating the novel N-terminus of Pr413. This region can be separately claimed.

As indicated by its expression profile, Pr413 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr413 aberrations includes, but is not limited to, prostate cancer.

Disorders associated with Pr413 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr413 expression can occur in cell types other than prostate, and thus can have a function outside of its role in prostate.

Nucleic acids of the present invention map to chromosomal band 6q23.1-q24.1. There are a number of different disorders which have been mapped to, or in close proximity to, this chromosome location. These include, e.g., Cardiomyopathy, SM2, IgA Nephropathy (IGAN), Oculodentodigital Dysplasia (ODDD), Argininemnia, Deafness, BCG infection, atypical, familial disseminated Mycobacterial infection, Zellweger syndrome, complementation group, myoclonic epilepsy, and transient neonatal diabetes.

Nucleic acids of the present invention can be used as linkage markers, diagnostic targets, therapeutic targets, for any of the mentioned disorders, as well as any disorders or genes mapping in proximity to it.

Pr426

Pr426 (related to NM_{—}001502) codes for a polypeptide containing 534 amino acids. It is also known as human pancreatic zymogen granule membrane glycoprotein-2 (GP2). The nucleotide and amino acid sequences of Pr426 are shown in SEQ ID NOS 51 and 52. It contains an endoglin/CD105 antigen domain at about amino acid positions 225-477, a transmembrane domain at about amino acid positions 511-533, and a signal peptide at about amino acid positions 1-27. A rat homolog is X53935.

As indicated by its expression profile, Pr426 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors.

Disorders associated with Pr426 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr426 expression can occur in cell types other than prostate, and thus can have a function outside of its role in prostate.

Nucleic acids of the present invention map to chromosomal band 9q21.11to q21.2. There are a number of different disorders which have been mapped to, or in close proximity to, this chromosome location. These include, e.g., Distal arthrogryposis-1, Deafness autosomal recessive 7, Geniospasm, Bleeding diathesis due to GNAQ deficiency, Choreoacanthocytosis, Ii blood group, and Friedreich ataxia Nucleic acids of the present invention can be used as linkage markers, diagnostic targets, therapeutic targets, for any of the mentioned disorders, as well as any disorders or genes mapping in proximity to it.

Pr449

Pr449 (related to AL137919 and AK026574) codes for a 546-amino acid protein-binding polypeptide. The nucleotide and amino acid sequences of Pr449 are shown in SEQ ID NOS 53 and 54. The polypeptide has six WD40 domains, at about amino acid positions 180-225, 233-269 281-319, 376-418, 424-461, and 471-506.

All or part of Pr449 is located in genomic DNA represented by AC012131 and BAC clone RP11-5E11. The present invention relates to any isolated introns and exons that are present in such clone. Such introns and exons can be routinely determined.

As indicated by its expression profile, Pr449 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr449 aberrations include, but are not limited to, prostate cancer and prostate adenocarcinoma.

Disorders associated with Pr449 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr449 expression can occur in cell types other than prostate, and thus can have a function outside of its role in prostate.

Nucleic acids of the present invention map to chromosomal band 10q11.22-q11.23. There are a number of different disorders which have been mapped to, or in close proximity to, this chromosome location. These include, e.g., Insulin-dependent diabetes mellitus, Cockayne syndrome, Thyroid papillary carcinoma, AIDS-resistance, Multiple endocrine neoplasia, Medullary thyroid carcinoma, Hirschsprung disease, and chronic infection due to opsonin defect. Nucleic acids of the present invention can be used as linkage markers, diagnostic targets, therapeutic targets, for any of the mentioned disorders, as well as any disorders or genes mapping in proximity to it.

Pr471

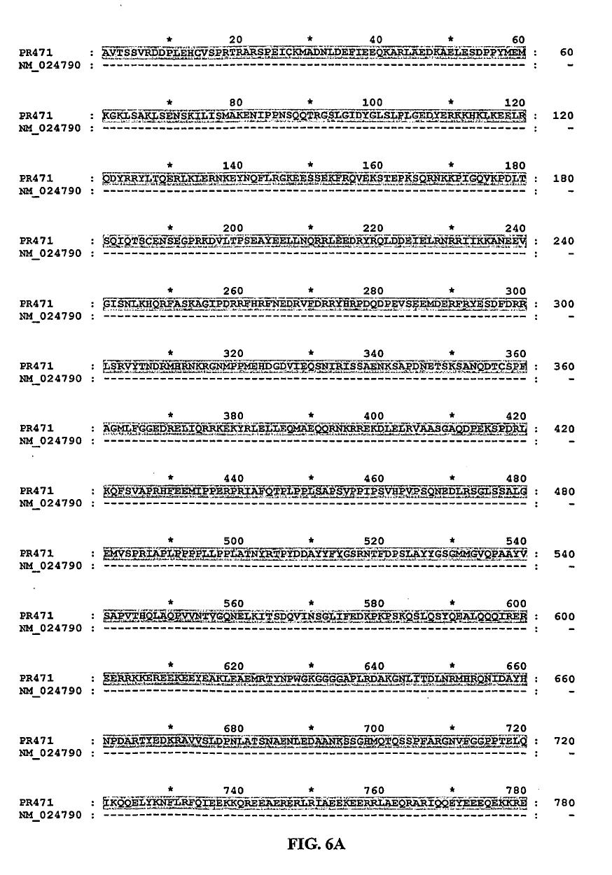

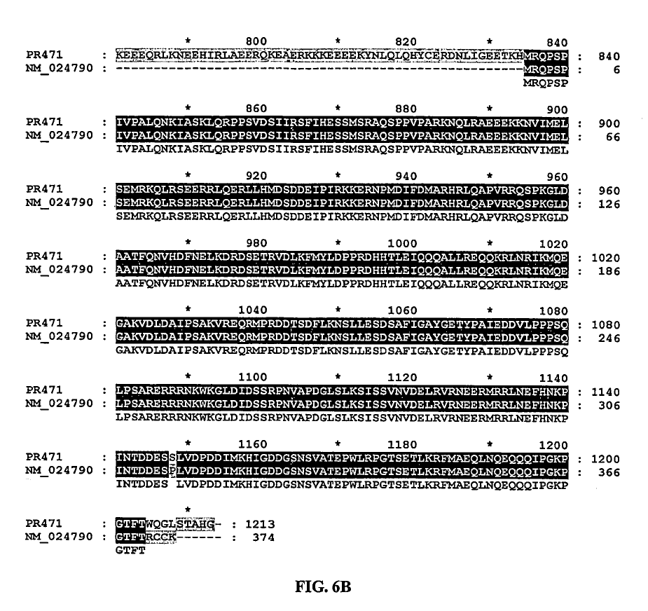

Pr471 (related to NM_{—}024790) codes for a nuclear regulatory factor containing 1185 amino. The nucleotide and amino acid sequences of Pr471 are shown in SEQ ID NOS 55 and 56. It contains six coiled-coil domains at about amino acid positions 78-99, 173-204, 347-378, 554-598, 701-793, and 854-894; a proline rich domain at amino acid positions 407-447; and a nuclear localization signal domain at 271-288. Alignment with NM_{—}024790 is shown in FIG. 6. Regions of variation, overlap, and non-overlap can be separately claimed.

All or part of Pr471 is located in genomic DNA represented by clones, AC079191, NT_{—}008616, AC02714, NT_{—}026463, and BAC clones RP11-436M3 and RP11-446H10. The gene contains at least 28 exons. The present invention relates to any isolated introns and exons that are present in such clone. Such introns and exons can be routinely determined.

As indicated by its expression profile, Pr471 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors.

Disorders associated with Pr471 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr471 expression can occur in cell types other than prostate, and thus can have a function outside of its role in prostate.

Nucleic acids of the present invention map to chromosomal band 8q13.1-13.3. There are a number of different disorders which have been mapped to, or in close proximity to, this chromosome location. These include, e.g., Ataxia with isolated vitamin E deficiency, Branchiootorenal syndrome, and Branchiootic syndrome. Nucleic acids of the present invention can be used as linkage markers, diagnostic targets, therapeutic targets, for any of the mentioned disorders, as well as any disorders or genes mapping in proximity to it.

Pr498

Pr498 codes for a transmembrane protein containing 354 amino acids. It has a signal polypeptide at about amino acid positions 1-40 and six transmembrane domains at about amino acid positions 44-66, 88-110, 122-144, 154-173, 194-213, and 233-250. The nucleotide and amino acid sequences of Pr498 are shown in SEQ ID NOS 57 and 58.

All or part of Pr498 is located in genomic DNA represented by AC069062, NT_{—}022370, and BAC clone RP11-399B17. The present invention relates to any isolated introns and exons that are present in such clone. Such introns and exons can be routinely determined.

As indicated by its expression profile, Pr498 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotype associated with Pr498 aberrations includes, but is not limited to, prostate cancer.

Disorders associated with Pr498 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr498 expression can occur in cell types other than prostate, and thus can have a function outside of its role in prostate.

Nucleic acids of the present invention map to chromosomal band 2q14.1. There are a number of different disorders which have been mapped to, or in close proximity to, this chromosome location. These include, e.g., Hypothyroidism, Purpura fulminans, Thrombophilia due to protein C deficiency, and Hepatocellular carcinoma. Nucleic acids of the present invention can be used as linkage markers, diagnostic targets, therapeutic targets, for any of the mentioned disorders, as well as any disorders or genes mapping in proximity to it.

Pr333

Pr333 codes for a transmembrane protein having 309 amino acid. The nucleotide and amino acid sequences of Pr333 are shown in SEQ ID NOS 67-68. It contains a signal polypeptide at about amino acids 1-20, eight transmembrane domains at about amino acid positions 34-53, 75-97, 102-121, 134-156, 171-193, 202-224, 239-261, and 274-296, and a UbiA domain at about amino acid positions 14-295. Polymorphisms for it are listed in Table 1.

All or part of Pr333 is located in genomic DNA represented by GenBank ID: AL031431/BAC-ID: RP3-462023, and Contig ID: NT_{—}004359. The present invention relates to any isolated introns and exons that are present in such clone. Such introns and exons can be routinely determined. The physical position of Pr333 is marked by UniSTS: 3646 at 5′ at 20.565 Mb and UniSTS: 5157 at,3′ at 20.616 Mb.

As indicated by its expression profile, Pr333 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The prostate phenotypes associated with Pr333 aberrations include, but are not limited to prostate cancer.

Disorders associated with Pr333 can affect as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr333 expression can occur in cell types other than prostate, and thus can have a function outside of its role in prostate.

Nucleic acids of the present invention map to chromosomal band 1p35.1-p36.12. There are a number of different disorders which have been mapped to, or in close proximity to, this chromosome location. These include, e.g., breast cancer; Inflammatory bowel disease; Melanoma; cutaneous malignant; CMM, Cataract, posterior polar; Parkinson disease; autosomal recessive early onset, Corneal dystrophy; Glaucoma 3, primary infantile B, etc.

Nucleic acids of the present invention can be used as linkage markers, diagnostic targets, therapeutic targets, for any of the mentioned disorders, as well as any disorders or genes mapping in proximity to it.

Pr410

Pr410 codes for a polypeptide containing 75 amino acids. The nucleotide and amino acid sequences of Pr410 are shown in SEQ ID NOS 69-70. It has a GGL domain at about amino acids 13-75. In addition, the 3′ UTR of the gene contains a transmembrane domain and TopC2 domain at nucleotide positions from 745 to 1059. Polymorphisms for it are listed in Table 1.

All or part of Pr410 is located in genomic DNA represented by GenBank ID: AL583844/BAC-ID: RP11-365D9, and Contig ID: NT_{—}004836. The present invention relates to any isolated introns and exons that are present in such clone. Such introns and exons can be routinely determined. The physical position of Pr410 is marked UniSTS: 592941 at 5′ at 244.770 Mb and UniSTS: 23794 at 3′ at 244.619 MB.

As indicated by its expression profile, Pr410 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors. The phenotypes associated with Pr410 aberrations include, but are not limited to prostate cancer, including a predisposition to it.

Disorders associated with Pr410 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr410 expression can occur in cell types other than prostate, and thus can have a function outside of its role in prostate.

Nucleic acids of the present invention map to chromosomal band 1q42.2-q43. There are a number of different disorders which have been mapped to, or in close proximity to, this chromosome location. These include, e.g., Leiomyoma, hereditary multiple of skin; Leiomyomatosis and renal cell cancer, hereditary; Hypoparathroidism-retardation-dysmorphism syndrome; Kenny-caffey syndrome type 1; Arrhythmogenic right ventricular dysplasia, familial. Nucleic acids of the present invention can be used as linkage markers, diagnostic targets, therapeutic targets, for any of the mentioned disorders, as well as any disorders or genes mapping in proximity to it.

Pr421

Pr421 codes for a ribosomal protein having 403 amino acids and a ribosomal protein domain at amino acid positions 1-296. The nucleotide and amino acid sequences of Pr421 are shown in SEQ ID NOS 71-72. Polymorphisms for it are listed in Table 1.

All or part of Pr421 is located in genomic DNA represented by GenBank ID: AL022326/BAC-ID: RP3-333H23, and Contig ID: NT_{—}011520.8. The present invention relates to any isolated introns and exons that are present in such clone. Such introns and exons can be routinely determined. The physical position of Pr421 is marked by UniSTS: 166519 at 5′ at 36.43 Mb and UniSTS: 16656 at 3′ at 36.423 Mb.

As indicated by its expression profile, Pr421 has a functional role in prostate. When the normal function of a gene is perturbed, the cells and tissues in which it is expressed are correspondingly affected, generally in a deleterious way. A range of different phenotypes are commonly observed, depending on the nature of the gene mutation and its interaction with other genetic and environmental factors.

Disorders associated with Pr421 can affect prostate, as well as other tissues and cell types in the body. Such gene effects can be caused by the direct action of the gene on another tissue or cell type, or indirectly, e.g., where a prostate tissue dysfunction or abnormality has downstream effects on other systems and cell types in the body. Furthermore, low levels of Pr421 expression can occur in cell types other than prostate, and thus can have a function outside of its role in prostate.

Nucleic acids of the present invention map to chromosomal band 22q13.1. There are a number of different disorders which have been mapped to, or in close proximity to, this chromosome location. These include, e.g., Colorectal cancer; Megakaryoblastic leukemia; Spinocerebellar ataxia; Waardenburg-Shah syndrome; Yemenite deaf-blind hypopigmentation syndrome; Parkinsonism susceptibility, and Debrisoquine sensitivity. Nucleic acids of the present invention can be used as linkage markers, diagnostic targets, therapeutic targets, for any of the mentioned disorders, as well as any disorders or genes mapping in proximity to it.

Genomic

The present invention also relates genomic DNA from which the polynucleotides of the present invention can be derived. A genomic DNA coding for a human, mouse, or other mammalian polynucleotide, can be obtained routinely, for example, by screening a genomic library (e.g., a YAC library) with a polynucleotide of the present invention, or by searching nucleotide databases, such as GenBank and EMBL, for matches. Promoter and other regulatory regions can be identified upstream of coding and expressed RNAs, and assayed routinely for activity, e.g., by joining to a reporter gene (e.g., CAT, GFP, alkaline phosphatase, luciferase, galatosidase). A promoter obtained from a prostate selective gene can be used, e.g., in gene therapy to obtain tissue-specific expression of a heterologous gene (e.g., coding for a therapeutic product or cytotoxin).

Constructs

A polynucleotide of the present invention can comprise additional polynucleotide sequences, e.g., sequences to enhance expression, detection, uptake, cataloging, tagging, etc. A polynucleofide can include only coding sequence; a coding sequence and additional non-naturally occurring or heterologous coding sequence (e.g., sequences coding for leader, signal, secretory, targeting, enzymatic, fluorescent, antibiotic resistance, and other functional or diagnostic peptides); coding sequences and non-coding sequences, e.g., untranslated sequences at either a 5′ or 3′ end, or dispersed in the coding sequence, e.g., introns.

A polynucleotide according to the present invention also can comprise an expression control sequence operably linked to a polynucleotide as described above. The phrase “expression control sequence” means a polynucleotide sequence that regulates expression of a polypeptide coded for by a polynucleotide to which it is functionally (“operably”) linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can include an initiation codon and additional nucleotides to place a partial nucleotide sequence of the present invention in-frame in order to produce a polypeptide (e.g., pET vectors from Promega have been designed to permit a molecule to be inserted into all three reading frames to identify the one that results in polypeptide expression). Expression control sequences can be heterologous or endogenous to the normal gene.

A polynucleotide of the present invention can also comprise nucleic acid vector sequences, e.g., for cloning, expression, amplification, selection, etc. Any effective vector can be used. A vector is, e.g., a polynucleotide molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60, pQE-9 (Qiagen), pBS, pD10, Phagescript, phiX174, pBK Phagemid, pNH8A, pNH16a, pNH18Z, pNH46A (Stratagene); Bluescript KS+II (Stratagene); ptrc99a, pKK223-3, pKK233-3, pDR54 0, pRIT5 (Pharmacia). Eukaryotic: PWLNEO, pSV2CAT, pOG44, pXT1, pSG (Stratagene), pSVK3, PBPV, PMSG, pSVL (Phannacia), pCR2.1/TOPO, pCRII/TOPO, pCR4/TOPO, pTrcHisB, pCMV6-XL4, etc. However, any other vector, e.g., plasmids, viruses, or parts thereof, may be used as long as they are replicable and viable in the desired host. The vector can also comprise sequences which enable it to replicate in the host whose genome is to be modified.

Hybridization

Polynucleotide hybridization, as discussed in more detail below, is useful in a variety of applications, including, in gene detection methods, for identifying mutations, for making mutations, to identify homologs in the same and different species, to identify related members of the same gene family, in diagnostic and prognostic assays, in therapeutic applications (e.g., where an antisense polynucleotide is used to inhibit expression), etc.

The ability of two single-stranded polynucleotide preparations to hybridize together is a measure of their nucleotide sequence complementarity, e.g., base-pairing between nucleotides, such as A-T, G-C, etc. The invention thus also relates to polynucleotides, and their complements, which hybridize to a polynucleotide comprising a nucleotide sequence as set forth in SEQ ID NOS 1-58 and 67-72 and genomic sequences thereof. A nucleotide sequence hybridizing to the latter sequence will have a complementary polynucleotide strand, or act as a template for one in the presence of a polymerase (i.e., an appropriate polynucleotide synthesizing enzyme). The present invention includes both strands of polynucleotide, e.g., a sense strand and an anti-sense strand.

Hybridization conditions can be chosen to select polynucleotides which have a desired amount of nucleotide complementarity with the nucleotide sequences set forth in SEQ ID NOS 1-58 and 67-72 and genomic sequences thereof. A polynucleotide capable of hybridizing to such sequence, preferably, possesses, e.g., about 70%, 75%, 80%, 85%, 87%, 90%, 92%, 95%, 97%, 99%, or 100% complementarity, between the sequences. The present invention particularly relates to polynucleotide sequences which hybridize to the nucleotide sequences set forth in SEQ ID NOS 1-58 and 67-72 or genomic sequences thereof, under low or high stringency conditions. These conditions can be used, e.g., to select corresponding homologs in non-human species.

Polynucleotides which hybridize to polynucleotides of the present invention can be selected in various ways. Filter-type blots (i.e., matrices containing polynucleotide, such as nitrocellulose), glass chips, and other matrices and substrates comprising polynucleotides (short or long) of interest, can be incubated in a prehybridization solution (e.g., 6×SSC, 0.5% SDS, 100 μg/ml denatured salmon sperm DNA, 5× Denhardt's solution, and 50% fonnamide), at 22-68° C., overnight, and then hybridized with a detectable polynucleotide probe under conditions appropriate to achieve the desired stringency. In general, when high homology or sequence identity is desired, a high temperature can be used (e.g., 65° C.). As the homology drops, lower washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, formamide can be omitted. See, e.g., Current Protocols in Molecular Biology, Chapter 6, Screening of Recombinant Libraries; Sambrook et al., Molecular Cloning, 1989, Chapter 9.

For instance, high stringency conditions can be achieved by incubating the blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity.

Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5+16.6log₁₀[Na⁺]+0.41(% GC)−600/N where [Na^{+] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.}

Stringent conditions can be selected to isolate sequences, and their complements, which have, e.g., at least about 90%, 95%, or 97%, nucleotide complementarity between the probe (e.g., a short polynucleotide of SEQ ID NOS 1-58 and 67-72 or genomic sequences thereof) and a target polynucleotide.

Other homologs of polynucleotides of the present invention can be obtained from mammalian and non-manunalian sources according to various methods. For example, hybridization with a polynucleotide can be employed to select homologs, e.g., as described in Sambrook et al., Molecular Cloning, Chapter 11, 1989. Such homologs can have varying amounts of nucleotide and amino acid sequence identity and similarity to such polynucleotides of the present invention. Mammalian organisms include, e.g., mice, rats, monkeys, pigs, cows, etc. Non-manumalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, artemia, viruses, etc. The degree of nucleotide sequence identity between human and mouse can be about, e.g. 70% or more, 85% or more for open reading frames, etc.

Alignment

Alignments can be accomplished by using any effective algorithm. For pairwise alignments of DNA sequences, the methods described by Wilbur-Lipman (e.g., Wilbur and Lipman, Proc. Natl. Acad. Sci., 80:726-730, 1983) or Martinez/Needleman-Wunsch (e.g., Martinez, Nucleic Acid Res., 11:4629-4634, 1983) can be used. For instance, if the Martinez/Needleman-Wunsch DNA alignment is applied, the minimum match can be set at 9, gap penalty at 1.10, and gap length penalty at 0.33. The results can be calculated as a similarity index, equal to the sum of the matching residues divided by the sum of all residues and gap characters, and then multiplied by 100 to express as a percent. Similarity index for related genes at the nucleotide level in accordance with the present invention can be greater than 70%, 80%, 85%, 90%, 95%, 99%, or more. Pairs of protein sequences can be aligned by the Lipman-Pearson method (e.g., Lipman and Pearson, Science, 227:1435-1441, 1985) with k-tuple set at 2, gap penalty set at 4, and gap length penalty set at 12. Results can be expressed as percent similarity index, where related genes at the amino acid level in accordance with the present invention can be greater than 65%, 70%, 75%, 80%, 85%, 90%, 95%, 99%, or more. Various commercial and free sources of alignment programs are available, e.g., MegAlign by DNA Star, BLAST (National Center for Biotechnology Information), BCM (Baylor College of Medicine) Launcher, etc. These calculations can be made along the entire length of each of the target sequences which are to be compared.

After two sequences have been aligned, a “percent sequence identity” can be determined. For these purposes, it is convenient to refer to a Reference Sequence and a Compared Sequence, where the Compared Sequence is compared to the Reference Sequence. Percent sequence identity can be determined according to the following formula: Percent Identity=100[1−(C/R)], wherein C is the number of differences between the Reference Sequence and the Compared Sequence over the length of alignment between the Reference Sequence and the Compared Sequence where (i) each base or amino acid in the Reference Sequence that does not have a corresponding aligned base or amino acid in the Compared Sequence, (ii) each gap in the Reference Sequence, (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

Percent sequence identity can also be determined by other conventional methods, e.g., as described in Altschul et al., Bull. Math. Bio. 48: 603-616, 1986 and Henikoff and Henikoff, Proc. Natl. Acad. Sci. USA 89:10915-10919, 1992.

Specific Polynucleotide Probes

A polynucleotide of the present invention can comprise any continuous nucleotide sequence of SEQ ID NOS 1-58 and 67-72, sequences which share sequence identity thereto, or complements thereof. The term “probe” refers to any substance that can be used to detect, identify, isolate, etc., another substance. A polynucleotide probe is comprised of nucleic acid can be used to detect, identify, etc., other nucleic acids, such as DNA and RNA.

These polynucleotides can be of any desired size that is effective to achieve the specificity desired. For example, a probe can be from about 7 or 8 nucleotides to several thousand nucleotides, depending upon its use and purpose. For instance, a probe used as a primer PCR can be shorter than a probe used in an ordered array of polynucleotide probes. Probe sizes vary, and the invention is not limited in any way by their size, e.g., probes can be from about 7-2000 nucleotides, 7-1000, 8-700, 8-600, 8-500, 8-400, 8-300, 8-150, 8-100, 8-75, 7-50, 10-25, 14-16, at least about 8, at least about 10, at least about 15, at least about 25, etc. The polynucleotides can have non-naturally-occurring nucleotides, e.g., inosine, AZT, 3TC, etc. The polynucleotides can have 100% sequence identity or complementarity to a sequence of SEQ ID NOS 1-58 and 67-72, or it can have mismatches or nucleotide substitutions, e.g., 1, 2, 3, 4, or 5 substitutions. The probes can be single-stranded or double-stranded.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids specific for genes differentially expressed in prostate, e.g., comprising a forward and reverse primer effective in PCR. These include both sense and anti-sense orientations. For instance, in PCR-based methods (such as RT-PCR), a pair of primers are typically used, one having a sense sequence and the other having an antisense sequence.

Another aspect of the present invention is a nucleotide sequence that is specific to, or for, a selective polynucleotide. The phrases “specific for” or “specific to” a polynucleotide have a functional meaning that the polynucleotide can be used to identify the presence of one or more target genes in a sample. It is specific in the sense that it can be used to detect polynucleotides above background noise (“non-specific binding”). A specific sequence is a defined order of nucleotides which occurs in the polynucleotide, e.g., in the nucleotide sequences of SEQ ID NOS 1-58 and 67-72. A probe or mixture of probes can comprise a sequence or sequences that are specific to a plurality of target sequences, e.g., where the sequence is a consensus sequence, a functional domain, etc., e.g., capable of recognizing a family of related genes. Such sequences can be used as probes in any of the methods described herein or incorporated by reference. Both sense and antisense nucleotide sequences are included. A specific polynucleotide according to the present invention can be determined routinely.

A polynucleotide comprising a specific sequence can be used as a hybridization probe to identify the presence of, e.g., human or mouse polynucleotide, in a sample comprising a mixture of polynucleotides, e.g., on a Northern blot. Hybridization can be performed under high stringent conditions (see, above) to select polynucleotides (and their complements which can contain the coding sequence) having at least 90%, 95%, 99%, etc., identity (i.e., complementarity) to the probe, but less stringent conditions can also be used. A specific polynucleotide sequence can also be fused in-frame, at either its 5′ or 3′ end, to various nucleotide sequences as mentioned throughout the patent, including coding sequences for enzymes, detectable markers, GFP, etc, expression control sequences, etc.

A polynucleotide probe, especially one that is specific to a polynucleotide of the present invention, can be used in gene detection and hybridization methods as already described. In one embodiment, a specific polynucleotide probe can be used to detect whether a particular tissue or cell-type is present in a target sample. To carry out such a method, a selective polynucleotide can be chosen which is characteristic of the desired target tissue. Such polynucleotide is preferably chosen so that it is expressed or displayed in the target tissue, but not in other tissues which are present in the sample. For instance, if detection of prostate is desired, it may not matter whether the selective polynucleotide is expressed in other tissues, as long as it is not expressed in cells normally present in blood, e.g., peripheral blood mononuclear cells. Starting from the selective polynucleotide, a specific polynucleotide probe can be designed which hybridizes (if hybridization is the basis of the assay) under the hybridization conditions to the selective polynucleotide, whereby the presence of the selective polynucleotide can be determined.

Probes which are specific for polynucleotides of the present invention can also be prepared using involve transcription-based systems, e.g., incorporating an RNA polymerase promoter into a selective polynucleotide of the present invention, and then transcribing anti-sense RNA using the polynucleotide as a template. See, e.g., U.S. Pat. No. 5,545,522.

Polynucleotide Composition

A polynucleotide according to the present invention can comprise, e.g., DNA, RNA, synthetic polynucleotide, peptide polynucleotide, modified nucleotides, dsDNA, ssDNA, ssRNA, dsRNA, and mixtures thereof. A polynucleotide can be single- or double-stranded, triplex, DNA:RNA, duplexes, comprise hairpins, and other secondary structures, etc. Nucleotides comprising a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Various modifications can be made to the polynucleotides, such as attaching detectable markers (avidin, biotin, radioactive elements, fluorescent tags and dyes, energy transfer labels, energy-emitting labels, binding partners, etc.) or moieties which improve hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967, 5,476,925, and 5,478,893.

Polynucleotide according to the present invention can be labeled according to any desired method. The polynucleotide can be labeled using radioactive tracers such as ³²P, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

Nucleic Acid Detection Methods

Another aspect of the present invention relates to methods and processes for detecting prostate tissues. Detection methods have a variety of applications, including for diagnostic, prognostic, forensic, and research applications. To accomplish gene detection, a polynucleotide in accordance with the present invention can be used as a “probe.” The term “probe” or “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence or absence, or it can be quantitative, e.g., in assessing amounts of a gene or gene transcript present in a sample. Probes can be useful in a variety of ways, such as for diagnostic purposes, to identify homologs, and to detect, quantitate, or isolate a polynucleotide of the present invention in a test sample.

Assays can be utilized which permit quantification and/or presence/absence detection of a target nucleic acid in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; WO97/18454; Prashar and Weissman, Proc. Natl. Acad. Sci., 93:659-663, and U.S. Pat. Nos. 6,010,850 and 5,712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. No. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl. Acad. Sci., 89, 3010-3014, 1992; U.S. Pat. No. 5,723,290). These and other methods can be carried out conventionally, e.g., as described in the mentioned publications.

Many of such methods may require that the polynucleotide is labeled, or comprises a particular nucleotide type useful for detection. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection.

Detection can be desirable for a variety of different purposes, including research, diagnostic, prognostic, and forensic. For diagnostic purposes, it may be desirable to identify the presence or quantity of a polynucleotide sequence in a sample, where the sample is obtained from tissue, cells, body fluids, etc. In a preferred method as described in more detail below, the present invention relates to a method of detecting a polynucleotide comprising, contacting a target polynucleotide in a test sample with a polynucleotide probe under conditions effective to achieve hybridization between the target and probe; and detecting hybridization.

Any test sample in which it is desired to identify a polynucleotide or polypeptide thereof can be used, including, e.g., blood, urine, saliva, stool (for extracting nucleic acid, see, e.g., U.S. Pat. No. 6,177,251), swabs comprising tissue, biopsied tissue, tissue sections, cultured cells, etc.

Detection can be accomplished in combination with polynucleotide probes for other genes, e.g., genes which are expressed in other disease states, tissues, cells, such as brain, heart, kidney, spleen, thymus, liver, stomach, small intestine, colon, muscle, lung, testis, placenta, pituitary, thyroid, skin, adrenal gland, pancreas, salivary gland, uterus, ovary, prostate gland, peripheral blood cells (T-cells, lymphocytes, etc.), embryo, normal breast, fat, adult and embryonic stem cells, specific cell-types, such as endothelial, epithelial, myocytes, adipose, luminal epithelial, basoepithelial, myoepithelial, stromal cells, etc.

Polynucleotides can be used in wide range of methods and compositions, including for detecting, diagnosing, staging, grading, assessing, prognosticating, etc. diseases and disorders associated with SEQ ID NOS 1-58 and 67-72, for monitoring or assessing therapeutic and/or preventative measures, in ordered arrays, etc. Any method of detecting genes and polynucleotides of SEQ ID NOS 1-58 and 67-72 can be used; certainly, the present invention is not to be limited how such methods are implemented.

Along these lines, the present invention relates to methods of detecting prostate tissue in a sample comprising nucleic acid. Such methods can comprise one or more the following steps in any effective order, e.g., contacting said sample with a polynucleotide probe under conditions effective for said probe to hybridize specifically to nucleic acid in said sample, and detecting the presence or absence of probe hybridized to nucleic acid in said sample, wherein said probe is a polynucleotide which is SEQ ID NOS 1-58 and 67-72, a polynucleotide having, e.g., about 70%, 80%, 85%, 90%, 95%, 99%, or more sequence identity thereto, effective or specific fragments thereof, or complements thereto. The detection method can be applied to any sample, e.g., cultured primary, secondary, or established cell lines, tissue biopsy, blood, urine, stool, and other bodily fluids, for any purpose.

Contacting the sample with probe can be carried out by any effective means in any effective environment. It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa.

Generally, as used throughout the specification, the term “effective conditions” means, e.g., the particular milieu in which the desired effect is achieved. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

The phrase “hybridize specifically” indicates that the hybridization between single-stranded polynucleotides is based on nucleotide sequence complementarity. The effective conditions are selected such that the probe hybridizes to a preselected and/or definite target nucleic acid in the sample. For instance, if detection of a polynucleotide set forth in SEQ ID NOS 1-58 and 67-72 is desired, a probe can be selected which can hybridize to such target gene under high stringent conditions, without significant hybridization to other genes in the sample. To detect homologs of a polynucleotide set forth in SEQ ID NOS 1-58 and 67-72, the effective hybridization conditions can be less stringent, and/or the probe can comprise codon degeneracy, such that a homolog is detected in the sample.

As already mentioned, the methods can be carried out by any effective process, e.g., by Northern blot analysis, polymerase chain reaction (PCR), reverse transcriptase PCR, RACE PCR, in situ hybridization, etc., as indicated above. When PCR based techniques are used, two or more probes are generally used. One probe can be specific for a defined sequence which is characteristic of a selective polynucleotide, but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

In addition to testing for the presence or absence of polynucleotides, the present invention also relates to determining the amounts at which polynucleotides of the present invention are expressed in sample and determining the differential expression of such polynucleotides in samples. Such methods can involve substantially the same steps as described above for presence/absence detection, e.g., contacting with probe, hybridizing, and detecting hybridized probe, but using more quantitative methods and/or comparisons to standards.

The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements. For further details, see the hybridization methods described above and below. Determining by such hybridization whether the target is differentially expressed (e.g., up-regulated or down-regulated) in the sample can also be accomplished by any effective means. For instance, the target's expression pattern in the sample can be compared to its pattern in a known standard, such as in a normal tissue, or it can be compared to another gene in the same sample. When a second sample is utilized for the comparison, it can be a sample of normal tissue that is known not to contain diseased cells. The comparison can be performed on samples which contain the same amount of RNA (such as polyadenylated RNA or total RNA), or, on RNA extracted from the same amounts of starting tissue. Such a second sample can also be referred to as a control or standard. Hybridization can also be compared to a second target in the same tissue sample. Experiments can be performed that determine a ratio between the target nucleic acid and a second nucleic acid (a standard or control), e.g., in a normal tissue. When the ratio between the target and control are substantially the same in a normal and sample, the sample is determined or diagnosed not to contain cells. However, if the ratio is different between the normal and sample tissues, the sample is determined to contain cancer cells. The approaches can be combined, and one or more second samples, or second targets can be used. Any second target nucleic acid can be used as a comparison, including “housekeeping” genes, such as beta-actin, alcohol dehydrogenase, or any other gene whose expression does not vary depending upon the disease status of the cell.

A goal, among others, of the method is to determine (i.e., identify) the presence of kidney tissue or cells in a sample of any origin. This can be accomplished by deciding whether one or more genes in a set of target genes are expressed in the sample of interest. Although the genes are selectively expressed in prostate, because of variability between individuals and tissue samples, each gene may not be expressed 100% of the time in prostate. There are many sources of variability that account for differences in gene penetrance, including, the state of the tissue and cells (e.g., normal, inflamed, diseased), cell cycle status, effects of other genes, environmental effects, age, health, gender, etc. Additionally, a selectively expressed gene may also be expressed in other tissue types. For instance, a selectively expressed gene can be expressed in multiple tissues, e.g., prostate and brain. Thus, expression of it in a sample indicates that the tissue is more likely to be either prostate or brain, than another tissue type, but this one probe is insufficient to distinguish between the two. For certain purposes, this level of certainty may be adequate. Determining that a second selectively nucleotide sequence for prostate is expressed in the sample provides greater certainty that the sample is prostate, not brain. For these and other reasons, certainty or probability that a given sample is kidney can be correlated with the number of selective genes expressed in the sample. Successive probes can be chosen based on their specificities. A greater number of genes determined to be expressed in a sample can indicate that there is a higher probability that the sample comprises prostate tissue. Probability values can be determined statistically and/or empirically, e.g., by making many measurements on individuals in a given population and determining the frequency in which the gene is expressed. These values can differ, depending upon the selected population, e.g., gender, health, ancestry, age, etc.

By the phrase “target genes,” it is meant the genes that the method is aimed at determining. Each of the nucleotide sequences shown in SEQ ID NOS 1-58 and 67-72 represents a region of a target gene, i.e., a fragment of a complete gene (e.g., a gene has regulatory and coding sequences) serving as a specific identification label for that target gene. The expression of the genes in a sample can be determined by any effective method. The term “expression” means, e.g., transcription of the gene into RNA, or translation of an RNA into protein. Expression can be determined, e.g., by detecting RNA, by detecting polypeptide translated from the RNA, or any product produced during expression of the gene. Nucleic acid and polypeptide detection are routine, and can be accomplished as described herein or as the skilled worker would know. For example, detecting of RNA can be performed by Northern blot analysis, polymerase chain reaction (PCR), reverse transcriptase PCR, RACE PCR, or in situ hybridization using a polynucleotide probe which is SEQ ID NOS 1-58 and 67-72, a polynucleotide having sequence identity thereto, effective specific fragments thereof, complements thereto, and said polynucleotide is selectively expressed in said kidney. Any amount of sequence identity is suitable as long as it maintains the desired amount of specificity.

Methods of Identifying Polymorphisms, Mutations, etc.

Polynucleotides of the present invention can also be utilized to identify mutant alleles, SNPs, gene rearrangements and modifications, and other polymorphisms of the wild-type gene. Mutant alleles, polymorphisms, SNPs, etc., can be identified and isolated from cancers that are known, or suspected to have, a genetic component. Identification of such genes can be carried out routinely (see, above for more guidance), e.g., using PCR, hybridization techniques, direct sequencing, mismatch reactions (see, e.g., above), RFLP analysis, SSCP (e.g., Orita et al., Proc. Natl. Acad. Sci., 86:2766, 1992), etc., where a polynucleotide having a sequence selected from SEQ ID NOS 1-58 and 67-72 is used as a probe. The selected mutant alleles, SNPs, polymorphisms, etc., can be used diagnostically to determine whether a subject has, or is susceptible to a disorder associated with SEQ ID NOS 1-58 and 67-72, as well as to design therapies and predict the outcome of the disorder. Methods involve, e.g., diagnosing a disorder associated with SEQ ID NOS 1-58 and 67-72, comprising, detecting the presence of a mutation in a gene represented by a polynucleotide selected from SEQ ID NOS 1-58 and 67-72. The detecting can be carried out by any effective method, e.g., obtaining cells from a subject, determining the gene sequence or structure of a target gene (using, e.g., mRNA, cDNA, genomic DNA, etc), comparing the sequence or structure of the target gene to the structure of the normal gene, whereby a difference in sequence or structure indicates a mutation in the gene in the subject. Polynucleotides can also be used to test for mutations, SNPs, polymorphisms, etc., e.g., using mismatch DNA repair technology as described in U.S. Pat. No. 5,683,877; U.S. Pat. No. 5,656,430; Wu et al., Proc. Natl. Acad. Sci., 89:8779-8783, 1992.

The present invention also relates to methods of detecting polymorphisms in SEQ ID NOS 1-58 and 67-72, comprising, e.g., comparing the structure of: genomic DNA comprising all or part of SEQ ID NOS 1-58 and 67-72, mRNA comprising all or part of SEQ ID NOS 1-58 and 67-72, cDNA comprising all or part of SEQ ID NOS 1-58 and 67-72, or a polypeptide comprising all or part of SEQ ID NOS 1-58 and 67-72, with the structure of SEQ ID NOS 1-58 and 67-72. The methods can be carried out on a sample from any source, e.g., cells, tissues, body fluids, blood, urine, stool, hair, egg, sperm, etc.

These methods can be implemented in many different ways. For example, “comparing the structure” steps include, but are not limited to, comparing restriction maps, nucleotide sequences, amino acid sequences, RFLPs, Dnase sites, DNA methylation fingerprints (e.g., U.S. Pat. No. 6,214,556), protein cleavage sites, molecular weights, electrophoretic mobilities, charges, ion mobility, etc., between a standard SEQ ID NOS 1-58 and 67-72 and a test SEQ ID NOS 1-58 and 67-72. The term “structure” can refer to any physical characteristics or configurations which can be used to distinguish between nucleic acids and polypeptides. The methods and instruments used to accomplish the comparing step depends upon the physical characteristics which are to be compared. Thus, various techniques are contemplated, including, e.g., sequencing machines (both amino acid and polynucleotide), electrophoresis, mass spectrometer (U.S. Pat. Nos. 6,093,541, 6,002,127), liquid chromatography, HPLC, etc.

To carry out such methods, “all or part” of the gene or polypeptide can be compared. For example, if nucleotide sequencing is utilized, the entire gene can be sequenced, including promoter, introns, and exons, or only parts of it can be sequenced and compared, e.g., exon 1, exon 2, etc.

Mutagenesis

Mutated polynucleotide sequences of the present invention are useful for various purposes, e.g., to create mutations of the polypeptides they encode, to identify functional regions of genomic DNA, to produce probes for screening libraries, etc. Mutagenesis can be carried out routinely according to any effective method, e.g., oligonucleotide-directed (Smith, M., Ann. Rev. Genet. 19:423-463, 1985), degenerate oligonucleotide-directed (Hill et al., Method Enzymology, 155:558-568, 1987), region-specific (Myers et al., Science, 229:242-246, 1985; Derbyshire et al., Gene, 46:145, 1986; Ner et al., DNA, 7:127, 1988), linker-scanning (McKnight and Kingsbury, Science, 217:316-324, 1982), directed using PCR, recursive ensemble mutagenesis (Arkin and Yourvan, Proc. Natl. Acad. Sci., 89:7811-7815, 1992), random mutagenesis (e.g., U.S. Pat. Nos. 5,096,815; 5,198,346; and 5,223,409), site-directed mutagenesis (e.g., Walder et al., Gene, 42:133, 1986; Bauer et al., Gene, 37:73, 1985; Craik, Bio Techniques, January 1985, 12-19; Smith et al., Genetic Engineering: Principles and Methods, Plenum Press, 1981), phage display (e.g., Lowman et al., Biochem. 30:10832-10837, 1991; Ladner et al., U.S. Pat. No. 5,223,409; Huse, WIPO Publication WO 92/06204), etc. Desired sequences can also be produced by the assembly of target sequences using mutually priming oligonucleotides (Uhlmann, Gene, 71:29-40, 1988). For directed mutagenesis methods, analysis of the three-dimensional structure of the SEQ ID NOS 1-58 and 67-72 polypeptide can be used to guide and facilitate making mutants which effect polypeptide activity. Sites of substrate-enzyme interaction or other biological activities can also be determined by analysis of crystal structure as determined by such techniques as nuclear magnetic resonance, crystallography or photoaffinity labeling. See, for example, de Vos et al., Science 255:306-312, 1992; Smith et al., J. Mol. Biol. 224:899-904,1992; Wlodaver et al., FEBS Lett. 309:59-64, 1992.

In addition, libraries of SEQ ID NOS 1-58 and 67-72 and fragments thereof can be used for screening and selection of SEQ ID NOS 1-58 and 67-72 variants. For instance, a library of coding sequences can be generated by treating a double-stranded DNA with a nuclease under conditions where the nicking occurs, e.g., only once per molecule, denaturing the double-stranded DNA, renaturing it to for double-stranded DNA that can include sense/antisense pairs from different nicked products, removing single-stranded portions from reformed duplexes by treatment with S1 nuclease, and ligating the resulting DNAs into an expression vecore. By this method, xpression libraries can be made comprising “mutagenized” SEQ ID NOS 1-58 and 67-72. The entire coding sequence or parts thereof can be used.

Polynucleotide Expression, Polypeptides Produced Thereby, and Specific-Binding Partners Thereto.

A polynucleotide according to the present invention can be expressed in a variety of different systems, in vitro and in vivo, according to the desired purpose. For example, a polynucleotide can be inserted into an expression vector, introduced into a desired host, and cultured under conditions effective to achieve expression of a polypeptide coded for by the polynucleotide, to search for specific binding partners. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding polynucleotide is adjacent to a dhfr gene), cycloheximide, cell densities, culture dishes, etc. A polynucleotide can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a polynucleotide of the present invention has been introduced is a transformed host cell. The polynucleotide can be extrachromosomal or integrated into a chromosome(s) of the host cell. It can be stable or transient. An expression vector is selected for its compatibility with the host cell. Host cells include, mammalian cells, e.g., COS, CV1, BHK, CHO, HeLa, LTK, NIH 3T3, PC-3 (CRL-1435), LNCaP (CRL-1740), CA-HPV-10 (CRL-2220), PZ-HPV-7 (CRL-2221), MDA-PCa 2b (CRL-2422), 22Rv1 (CRL2505), NCI-H660 (CRL-5813), HS 804.Sk (CRL-7535), LNCaP-FGF (CRL-10995), RWPE-1 (CRL-11609), RWPE-2 (CRL-11610), PWR-1E (CRL 11611), rat MAT-Ly-LuB-2 (CRL-2376), etc., insect cells, such as Sf9 (S. frugipeda) and Drosophila, bacteria, such as E. coli, Streptococcus, bacillus, yeast, such as Sacharomyces, S. cerevisiae, fingal cells, plant cells, embryonic or adult stem cells (e.g., mammalian, such as mouse or human).

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, MMTV, SV40, trp, lac, tac, or T7 promoters for bacterial hosts; or alpha factor, alcohol oxidase, or PGH promoters for yeast. RNA promoters can be used to produced RNA transcripts, such as T7 or SP6. See, e.g., Melton et al., Polyniucleotide Res., 12(18):7035-7056, 1984; Dunn and Studier. J. Mol. Bio., 166:477-435, 1984; U.S. Pat. No.5,891,636; Studier et al., Genie Expression Technology, Methods in Enzymology, 85:60-89, 1987. In addition, as discussed above, translational signals (including in-frame insertions) can be included.

When a polynucleotide is expressed as a heterologous gene in a transfected cell line, the gene is introduced into a cell as described above, under effective conditions in which the gene is expressed. The term “heterologous” means that the gene has been introduced into the cell line by the “hand-of-man.” Introduction of a gene into a cell line is discussed above. The transfected (or transformed) cell expressing the gene can be lysed or the cell line can be used intact.

For expression and other purposes, a polynucleotide can contain codons found in a naturally-occurring gene, transcript, or cDNA, for example, e.g., as set forth in SEQ ID NOS 1-58 and 67-72, or it can contain degenerate codons coding for the same amino acid sequences. For instance, it may be desirable to change the codons in the sequence to optimize the sequence for expression in a desired host. See, e.g., U.S. Pat. Nos. 5,567,600 and 5,567,862.

A polypeptide according to the present invention can be recovered from natural sources, transformed host cells (culture medium or cells) according to the usual methods, including, detergent extraction (e.g., non-ionic detergent, Triton X-100, CHAPS, octylglucoside, Igepal CA-630), ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis. Protein refolding steps can be used, as necessary, in completing the configuration of the mature protein. Finally, high performance liquid chromatography (HPLC) can be employed for purification steps. Another approach is express the polypeptide recombinantly with an affinity tag (Flag epitope, HA epitope, myc epitope, 6xHis, maltose binding protein, chitinase, etc) and then purify by anti-tag antibody-conjugated affinity chromatography.

The present invention also relates to antibodies, and other specific-binding partners, which are specific for polypeptides encoded by polynucleotides of the present invention, e.g., SEQ ID NOS 1-58 and 67-72. Antibodies, e.g., polyclonal, monoclonal, recombinant, chimeric, humanized, single-chain, Fab, and fragments thereof, can be prepared according to any desired method. See, also, screening recombinant imnmunoglobulin libraries (e.g., Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. The antibodies can be IgM, IgG, subtypes, IgG2a, IgG1, etc. Antibodies, and immune responses, can also be generated by administering naked DNA See, e.g., U.S. Pat. Nos. 5,703,055; 5,589,466; 5,580,859. Antibodies can be used from any source, including, goat, rabbit, mouse, chicken (e.g., IgY; see, Duan, W0/029444 for methods of making antibodies in avian hosts, and harvesting the antibodies from the eggs). An antibody specific for a polypeptide means that the antibody recognizes a defined sequence of amino acids within or including the polypeptide. Other specific binding partners include, e.g., aptamers and PNA. antibodies can be prepared against specific epitopes or domains of SEQ ID NOS 1-58 and 67-72.

The preparation of polyclonal antibodies is well-known to those skilled in the art. See, for example, Green et al., Production of Polyclonal Antisera, in IMMUNOCHEMICAL PROTOCOLS (Manson, ed.), pages 1-5 (Humana Press 1992); Coligan et al., Production of Polyclonal Antisera in Rabbits, Rats, Mice and Hamsters, in CURRENT PROTOCOLS IN IMMUNOLOGY, section 2.4.1 (1992). The preparation of monoclonal antibodies likewise is conventional. See, for example, Kohler & Milstein, Nature 256:495 (1975); Coligan et al., sections 2.5.1-2.6.7; and Harlow et al., ANTIBODIES: A LABORATORY MANUAL, page 726 (Cold Spring Harbor Pub. 1988).

Antibodies can also be humanized, e.g., where they are to be used therapeutically. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989), which is hereby incorporated in its entirety by reference. Techniques for producing humanized monoclonal antibodies are described, for example, in U.S. Pat. No. 6,054,297, Jones et al., Nature 321: 522 (1986); Riechmann et al., Nature 332: 323 (1988); Verhoeyen et al., Science 239: 1534 (1988); Carter et al., Proc. Nat'l Acad. Sci. USA 89: 4285 (1992); Sandhu, Crit. Rev. Biotech. 12: 437 (1992); and Singer et al., J. Immunol. 150: 2844 (1993).

Antibodies of the invention also maybe derived from human antibody fragments isolated from a combinatorial immunoglobulin library. See, for example, Barbas et al., METHODS: A COMPANION TO METHODS IN ENZYMOLOGY, VOL. 2, page 119 (1991); Winter et al., Ann. Rev. Immunol. 12: 433 (1994). Cloning and expression vectors that are useful for producing a human immunoglobulin phage library can be obtained commercially, for example, from STRATAGENE Cloning Systems (La Jolla, Calif.).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens and can be used to produce human antibody-secreting hybridomas. Methods for obtaining human antibodies from transgenic mice are described, e.g., in Green et al., Nature Genet. 7:13 (1994); Lonberg et al., Nature 368:856 (1994); and Taylor et al., Int. Inmunol. 6:579 (1994).

Antibody fragments of the present invention can be prepared by proteolytic hydrolysis of the antibody or by expression in E. coli of nucleic acid encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′).sub.2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. No. 4,036,945 and No. 4,331,647, and references contained therein. These patents are hereby incorporated in their entireties by reference. See also Nisoiihoff et al., Arch. Biochem. Biophys. 89:230 (1960); Porter, Biochem. J. 73:119 (1959); Edelman etal, METHODS IN ENZYMOLOGY, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques can also be used. For example, Fv fragments comprise an association of V.sub.H and V.sub.L chains. This association may be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, e.g., Sandhu, supra. Preferably, the Fv fragments comprise V.sub.H and V.sub.L chains connected by a peptide linker. These single-chain antigen binding proteins (sFv) are prepared by constructing a structural gene comprising nucleic acid sequences encoding the V.sub.H and V.sub.L domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow et al., METHODS: A COMPANION TO METHODS IN ENZYMOLOGY, VOL. 2, page 97 (1991); Bird etal.,Science 242:423-426 (1988); Ladneret al., U.S. Pat. No. 4,946,778; Pack et al., Bio/Technology 11: 1271-77 (1993); and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., METHODS: A COMPANION TO METHODS IN ENZYMOLOGY, VOL. 2, page 106 (1991).

The term “antibody” as used herein includes intact molecules as well as fragments thereof, such as Fab, F(ab′)2, and Fv which are capable of binding to an epitopic determinant present in Bin1 polypeptide. Such antibody fragments retain some ability to selectively bind with its antigen or receptor. The term “epitope” refers to an antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. Antibodies can be prepared against specific epitopes or polypeptide domains.

Antibodies which bind to SEQ ID NOS 1-58 and 67-72 polypeptides of the present invention can be prepared using an intact polypeptide or fragments containing small peptides of interest as the immunizing antigen. For example, it maybe desirable to produce antibodies that specifically bind to the N- or C-terminal domains of SEQ ID NOS 1-58 and 67-72. The polypeptide or peptide used to immunize an animal which is derived from translated cDNA or chemically synthesized which can be conjugated to a carrier protein, if desired. Such commonly used carriers which are chemically coupled to the immunizing peptide include keyhole limpet hemocyanin (KLH), thyroglobulin, bovine serum albumin (BSA), and tetanus toxoid.

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1994, incorporated by reference).

Anti-idiotype technology can also be used to produce invention monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

Methods of Detecting Polypeptides

Polypeptides coded for by SEQ ID NOS 1-58 and 67-72 of the present invention can be detected, visualized, determined, quantitated, etc. according to any effective method. useful methods include, e.g., but are not limited to, immunoassays, RIA (radioimmunassay), ELISA, (enzyme-linked-immunosorbent assay), immunoflourescence, flow cytometry, histology, electron microscopy, light microscopy, in situ assays, immunoprecipitation, Western blot,

Immunoassays may be carried in liquid or on biological support. For instance, a sample (e.g., blood, stool, urine, cells, tissue, body fluids, etc.) can be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled SEQ ID NOS 1-58 and 67-72 specific antibody. The solid phase support can then be washed with a buffer a second time to remove unbound antibody. The amount of bound label on solid support may then be detected by conventional means.

A “solid phase support or carrier” includes any support capable of binding an antigen, antibody, or other specific binding partner. Supports or carriers include glass, polystyrene, polypropylene, polyethylene, dextran, nylon, amylases, natural and modified celluloses, polyacrylamides, and magnetite. A support material can have any structural or physical configuration. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads.

One of the many ways in which gene peptide-specific antibody can be detectably labeled is by linking it to an enzyme and using it in an enzyme immunoassay (EIA). See, e.g., Voller, A., “The Enzyme Linked Immunosorbent Assay (ELISA),” 1978, Diagnostic Horizons 2, 1-7, Microbiological Associates Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla. The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staplhylococcal nuclease, delta-5-steroid isomerase, yeast alcohol dehydrogenase, .alpha.-glycerophosphate, dehydrogenase, triose phosphate isomerase, horseradish peroxidase, alkaline phosphatase, asparaginase, glucose oxidase, beta.-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect SEQ ID NOS 1-58 and 67-72 peptides through the use of a radioimmunoassay (RIA). See, e.g., Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerytlirin, phycocyanin, allophycocyanin, o-phthaldehyde and fluorescamine. The antibody can also be detectably labeled using fluorescence emitting metals such as those in the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the cheriluminescent reaction. The presence of a bioluminescent protein is determined by detecting the presence of luminescence. Important bioluminescent compounds for purposes of labeling are luciferin, luciferase and aequorin.

Diagnostic

The present invention also relates to methods and compositions for diagnosing a prostate disorder using polynucleotides, polypeptides, and specific-binding partners of the present invention to detect, assess, determine, etc., SEQ ID NOS 1-58 and 67-72. In such methods, the gene can serve as a marker for the disorder, e.g., where the gene, when mutant, is a direct cause of the disorder; where the gene is affected by another gene(s) which is directly responsible for the disorder, e.g., when the gene is part of the same signaling pathway as the directly responsible gene; and, where the gene is chromosomally linked to the gene(s) directly responsible for the disorder, and segregates with it. Many other situations are possible. To detect, assess, determine, etc., a probe specific for the gene can be employed as described above and below. Any method of detecting and/or assessing the gene can be used, including detecting expression of the gene using polynucleotides, antibodies, or other specific-binding partners.

The present invention relates to methods of diagnosing a prostate disorders, comprising, e.g., assessing the expression of SEQ ID NOS 1-58 and 67-72 in a tissue sample comprising tissue or cells suspected of having the disorder (e.g., where the sample comprises prostate). The phrase “diagnosing” indicates that it is determined whether the sample has the disorder. A “disorder” means, e.g., any abnormal condition as in a disease or malady.

By the phrase “assessing expression of SEQ ID NOS 1-58 and 67-72,” it is meant that the functional status of the gene is evaluated. This includes, but is not limited to, measuring expression levels of said gene, determining the genomic structure of said gene, determining the mRNA structure of transcripts from said gene, or measuring the expression levels of polypeptide coded for by said gene. Thus, the term “assessing expression” includes evaluating the all aspects of the transcriptional and translational machinery of the gene. For instance, if a promoter defect causes, or is suspected of causing, the disorder, then a sample can be evaluated (i.e., “assessed”) by looking (e.g., sequencing or restriction mapping) at the promoter sequence in the gene, by detecting transcription products (e.g., RNA), by detecting translation product (e.g., polypeptide). Any measure of whether the gene is functional can be used, including, polypeptide, polynucleotide, and functional assays for the gene's biological activity.

In making the assessment, it can be useful to compare the results to a normal gene, e.g., a gene which is not associated with the disorder. The nature of the comparison can be determined routinely, depending upon how the assessing is accomplished. If, for example, the mRNA levels of a sample is detected, then the mRNA levels of a normal can serve as a comparison, or a gene which is known not to be affected by the disorder. Methods of detecting mRNA are well known, and discussed above, e.g., but not limited to, Northern blot analysis, polymerase chain reaction (PCR), reverse transcriptase PCR, RACE PCR, etc. Similarly, if polypeptide production is used to evaluate the gene, then the polypeptide in a normal tissue sample can be used as a comparison, or, polypeptide from a different gene whose expression is known not to be affected by the disorder. These are only examples of how such a method could be carried out.

Assessing the effects of therapeutic and preventative interventions (e.g., administration of a drug, chemotherapy, radiation, etc.) on prostate disorders is a major effort in drug discovery, clinical medicine, and pharmacogenomics. The evaluation of therapeutic and preventative measures, whether experimental or already in clinical use, has broad applicability, e.g., in clinical trials, for monitoring the status of a patient, for analyzing and assessing animal models, and in any scenario involving cancer treatment and prevention. Analyzing the expression profiles of polynucleotides of the present invention can be utilized as a parameter by which interventions are judged and measured. Treatment of a disorder can change the expression profile in some manner which is prognostic or indicative of the drug's effect on it. Changes in the profile can indicate, e.g., drug toxicity, return to a normal level, etc. Accordingly, the present invention also relates to methods of monitoring or assessing a therapeutic or preventative measure (e.g., chemotherapy, radiation, anti-neoplastic drugs, antibodies, etc.) in a subject having a prostate disorder, or, susceptible to such a disorder, comprising, e.g., detecting the expression levels of SEQ ID NOS 1-58 and 67-72. A subject can be a cell-based assay system, non-human animal model, human patient, etc. Detecting can be accomplished as described for the methods above and below. By “therapeutic or preventative intervention,” it is meant, e.g., a drug administered to a patient, surgery, radiation, chemotherapy, and other measures taken to prevent, treat, or diagnose a disorder.

Polynucleotides of the present invention can be used to identify, detect, stage, determine the presence of, prognosticate, treat, study, etc., diseases and conditions of the prostate. such as prostate cancer.

Polynucleotides can also be used for staging and classifying conditions and diseases of the present invention, alone, or in combination with conventional staging and classification schemes.

As discussed elsewhere, expression can be assessed in any sample comprising any tissue or cell type, body fluid, etc., including cells from prostate can be used, or cells derived from prostate. By the phrase “cells derived from prostate,” it is meant that the derived cells originate from prostate, e.g., when metastasis from a primary tumor site has occurred, when a progenitor-type or pluripotent cell gives rise to other cells, etc.

Identifying Agent Methods

The present invention also relates to methods of identifying agents, and the agents themselves, which modulate differentially regulated genes and gene products of the present invention. These agents can be used to modulate the biological activity of the polypeptide encoded for the gene, or the gene, itself. Agents which regulate the gene or its product are useful in variety of different environments, including as medicinal agents to treat or prevent disorders associated with differentially regulated genes and as research reagents to modify the function of tissues and cell.

Methods of identifying agents generally comprise steps in which an agent is placed in contact with the gene, transcription product, translation product, or other target, and then a determination is performed to assess whether the agent “modulates” the target. The specific method utilized will depend upon a number of factors, including, e.g., the target (i.e., is it the gene or polypeptide encoded by it), the environment (e.g., in vitro or in vivo), the composition of the agent, etc.

For modulating the expression of differentially-regulated genes of the present invention, a method can comprise, in any effective order, one or more of the following steps, e.g., contacting a differentially-regulated gene (e.g., in a cell population) with a test agent under conditions effective for said test agent to modulate the expression of said gene, and determining whether said test agent modulates said gene. An agent can modulate expression of a differentially-regulated gene at any level, including transcription, translation, and/or perdurance of the nucleic acid (e.g., degradation, stability, etc.) in the cell. For modulating the biological activity of polypeptides coded for by differentially-regulated genes, a method can comprise, in any effective order, one or more of the following steps, e.g., contacting a polypeptide (e.g., in a cell, lysate, or isolated) with a test agent under conditions effective for said test agent to modulate the biological activity of said polypeptide, and determining whether said test agent modulates said biological activity.

Contacting a differentially-regulated gene or polypeptide with the test agent can be accomplished by any suitable method and/or means that places the agent in a position to functionally control expression or biological activity of said gene or polypeptide present in the sample. Functional control indicates that the agent can exert its physiological effect on the gene or polypeptide through whatever mechanism it works. The choice of the method and/or means can depend upon the nature of the agent and the condition and type of environment in which the gene or polypeptide is presented, e.g., lysate, isolated, or in a cell population (such as, in vivo, in vitro, organ explants, etc.). For instance, if the cell population is an in vitro cell culture, the agent can be contacted with the cells by adding it directly into the culture medium. If the agent cannot dissolve readily in an aqueous medium, it can be incorporated into liposomes, or another lipophilic carrier, and then administered to the cell culture. Contact can also be facilitated by incorporation of agent with carriers and delivery molecules and complexes, by injection, by infusion, etc.

After the agent has been administered in such a way that it can gain access to the gene or polypeptide, it can be determined whether the test agent modulates their expression or biological activity. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate gene expression means, e.g., that the test agent has an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc. To modulate biological activity means, e.g., that a functional activity of the polypeptide is changed in comparison to its normal activity in the absence of the agent. This effect includes, increase, decrease, block, inhibit, enhance, etc.

A test agent can be of any molecular composition, e.g., chemical compounds, biomolecules, such as polypeptides, lipids, nucleic acids (e.g., antisense to a polynucleotide sequence), carbohydrates, antibodies, ribozymes, double-stranded RNA, aptamers, etc. For example, if a polypeptide to be modulated is a cell-surface molecule, a test agent can be an antibody that specifically recognizes it and, e.g., causes the polypeptide to be internalized, leading to its down regulation on the surface of the cell. Such an effect does not have to be permanent, but can require the presence of the antibody to continue the down-regulatory effect. Antibodies can also be used to modulate the biological activity a polypeptide in a lysate or other cell-free form. Antisense can also be used as test agents to modulate gene expression.

Markers

The polynucleotides of the present invention can be used with other markers, especially prostate markers, to identity, detect, stage, diagnosis, determine, prognosticate, treat, etc., tissue, diseases and conditions, etc, of the prostate. Markers can be polynucleotides, polypeptides, antibodies, ligands, specific binding partners, etc. The targets for such markers include, but are not limited genes and polypeptides that are selective for cell types present in the prostate. A number of genes and gene products have been identified which are associated with prostate cancer metastasis and/or progression, e.g., PSA, KAI1 (shows decreased expression in metastatic cells; Dong et al., Science, 268:884-6, 1995), D44 isoforms (differentially-regulated during carcinoma progression; Noordzij et al., Clin. Cancer Res., 3:805-15, 1997), p53 (Effert et al., J. Urol., 150:257-61, 1993), Rb, CDKN2, E-cadherin, PTEN (Hamilton et al., Br. J. Cancer, 82:1671-6, 2000; Dong et al., Clin. Cancer Res., 7:304-308, 2001), bc1-2, prostatic acid phosphatase (PAP), prostate specific membrane antigen (e.g., U.S. Pat. Nos. 5,538,866 and 6,107,090), Smad3 (e.g., Kang et al., Proc. Natl. Acad. Sci., 98:3018-3023, 2001), TGF-beta, and other oncogenes and tumor suppressor genes. See, also, Myers and Grizzle, Eur. Urol., 30:153-166, 1996, for other biomarkers associated with prostatic carcinoma, such as PCNA, p185-erbB-2, p180erbB-3, TAG-72, nm23-H1 and FASE.

Therapeutics

Selective polynucleotides, polypeptides, and specific-binding partners thereto, can be utilized in therapeutic applications, especially to treat diseases and conditions of prostate. Useful methods include, but are not limited to, immunotherapy (e.g., using specific-binding partners to polypeptides), vaccination (e.g., using a selective polypeptide or a naked DNA encoding such polypeptide), protein or polypeptide replacement therapy, gene therapy (e.g., germ-line correction, antisense), etc.

Various immunotherapeutic approaches can be used. For instance, unlabeled antibody that specifically recognizes a tissue-specific antigen can be used to stimulate the body to destroy or attack the cancer, to cause down-regulation, to produce complement-mediated lysis, to inhibit cell growth, etc., of target cells which display the antigen, e.g., analogously to how c-erbB-2 antibodies are used to treat breast cancer. In addition, antibody can be labeled or conjugated to enhance its deleterious effect, e.g., with radionuclides and other energy emitting entitities, toxins, such as ricin, exotoxin A (ETA), and diphtheria, cytotoxic or cytostatic agents, immunomodulators, chemotherapeutic agents, etc. See, e.g., U.S. Pat. No. 6,107,090.

An antibody or other specific-binding partner can be conjugated to a second molecule, such as a cytotoxic agent, and used for targeting the second molecule to a tissue-antigen positive cell (Vitetta, E. S. et al., 1993, Immunotoxin therapy, in DeVita, Jr., V. T. et al., eds, Cancer: Principles and Practice of Oncology, 4th ed., J. B. Lippincott Co., Philadelphia, 2624-2636). Examples of cytotoxic agents include, but are not limited to, antimetabolites, alkylating agents, anthracyclines, antibiotics, anti-mitotic agents, radioisotopes and chemotherapeutic agents. Further examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, 1-dehydrotestosterone, diptheria toxin, Pseudomonas exotoxin (PE) A, PE40, abrin, elongation factor-2 and glucocorticoid. Techniques for conjugating therapeutic agents to antibodies are well.

In addition to inununotherapy, polynucleotides and polypeptides can be used as targets for non-immunotherapeutic applications, e.g., using compounds which interfere with function, expression (e.g., antisense as a therapeutic agent), assembly, etc. RNA interference can be used in vivtro and in vivo to silence SEQ ID NOS 1-58 and 67-72 when its expression contributes to a disease (but also for other purposes, e.g., to identify the gene's function to change a developmental pathway of a cell, etc.). See, e.g., Sharp and Zarnore, Science, 287:2431-2433, 2001; Grishok et al., Science, 287:2494, 2001.

Delivery of therapeutic agents can be achieved according to any effective method, including, liposomes, viruses, plasmid vectors, bacterial delivery systems, orally, systemically, etc.

Antisense

Antisense polynucleotide (e.g., RNA) can also be prepared from a polynucleotide according to the present invention, preferably an anti-sense to a sequence of SEQ ID NOS 1-58 and 67-72. Antisense polynucleotide can be used in various ways, such as to regulate or modulate expression of the polypeptides they encode, e.g., inhibit their expression, for in situ hybridization, for therapeutic purposes, for making targeted mutations (in vivo, triplex, etc.) etc. For guidance on administering and designing anti-sense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095, 5,998,383, 5,994,230, 5,891,725, 5,885,970, and 5,840,708. An antisense polynucleotides can be operably linked to an expression control sequence. A total length of about 35 bp can be used in cell culture with cationic liposomes to facilitate cellular uptake, but for in vivo use, preferably shorter oligonucleotides are administered, e.g. 25 nucleotides.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); 4,973,679; Sproat et al., “2′-O-Methyloligoribonucleotides: synthesis and applications,” Oligonucleotides and Analogs A Practical Approach, Eckstein (ed.), IRL Press, Oxford, 1991, 49-86; Iribarren et al., “2′O-Alkyl Oligoribonucleotides as Antisense Probes,” Proc. Natl. Acad. Sci. USA, 1990, 87, 7747-7751; Cotton et al., “2′-O-methyl, 2′-O-ethyl oligoribonucleotides and phosphorothioate oligodeoxyribonucleotides as inhibitors of the in vitro U7 snRNP-dependent mRNA processing event,” Nucl. Acids Res., 1991, 19, 2629-2635.

Arrays

The present invention also relates to an ordered array of polynucleotide probes and specific-binding partners (e.g., antibodies) for detecting the expression of a polynucleotide selected from SEQ ID NOS 1-58 and 67-72 in a sample, or a polypeptide encoded thereby, comprising, one or more polynucleotide probes or specific binding partners associated with a solid support, wherein each probe is specific for a polynucleotide selected from SEQ ID NOS 1-58 and 67-72, or a polypeptide encoded therby, and the probes comprise a nucleotide sequence selected from SEQ ID NOS 1-58 and 67-72 which is specific for said gene, a nucleotide sequence having sequence identity to a polynucleotide selected from SEQ ID NOS 1-58 and 67-72 and which is specific for said polynucleotide, or complements thereto, or a specific-binding partner which is specific for a polypeptide coded for by the a polynucleotide selected from SEQ ID NOS 1-58 and 67-72.

The phrase “ordered array” indicates that the probes are arranged in an identifiable or position-addressable pattern, e.g., such as the arrays disclosed in U.S. Pat. Nos. 6,156,501, 6,077,673, 6,054,270, 5,723,320, 5,700,637, W009919711, W000023803. The probes are associated with the solid support in any effective way. For instance, the probes can be bound to the solid support, either by polymerizing the probes on the substrate, or by attaching a probe to the substrate. Association can be, covalent, electrostatic, noncovalent, hydrophobic, hydrophilic, noncovalent, coordination, adsorbed, absorbed, polar, etc. When fibers or hollow filaments are utilized for the array, the probes can fill the hollow orifice, be absorbed into the solid filament, be attached to the surface of the orifice, etc. Probes can be of any effective size, sequence identity, composition, etc., as already discussed.

Ordered arrays can further comprise polynucleotide probes or specific-binding partners which are specific for other genes, including genes specific for prostate or disorders associated with prostate.

Transgenic Animals

The present invention also relates to transgenic animals comprising a modified gene of the present invention, e.g., a polynucleotide selected from SEQ ID NOS 1-58 and 67-72, and homologs thereof, e.g., homologs from mice. Such genes, as discussed in more detail below, include, but are not limited to, functionally-disrupted genes, mutated genes, ectopically or selectively-expressed genes, inducible or regulatable genes, etc. These transgenic animals can be produced according to any suitable technique or method, including homologous recombination, mutagenesis (e.g., ENU, Rathkolb et al., Exp. Physiol., 85(6):635-644, 2000), and the tetracycline-regulated gene expression system (e.g., U.S. Pat. No. 6,242,667). The term “gene” as used herein includes any part of a gene, i.e., regulatory sequences, promoters, enhancers, exons, introns, coding sequences, etc. A polynucleotide of SEQ ID NOS 1-58 and 67-72, or a homolog thereof, which is present in the construct or transgene can be naturally-occurring wild-type, polymorphic, or mutated. Transgenic animals in accordance with the present invention can be susceptible to prostate and other diseases which are mentioned above. In addition, polynucleotides of the present invention can be used to prevent or treat such diseases and conditions.

Along these lines, polynucleotides of the present invention can be used to create transgenic animals, e.g. a non-human animal, comprising at least one cell whose genome comprises a functional disruption of a polynucleotide selected from SEQ ID NOS 1-58 and 67-72, or a homolog thereof. By the phrases “functional disruption” or “functionally disrupted,” it is meant that the gene does not express a biologically-active product. It can be substantially deficient in at least one functional activity coded for by the gene. Expression of a polypeptide can be substantially absent, i.e., essentially undetectable amounts are made. However, polypeptide can also be made, but which is deficient in activity, e.g., where only an amino-terminal portion of the gene product is produced.

The transgenic animal can comprise one or more cells. When substantially all its cells contain the engineered gene, it can be referred to as a transgenic animal “whose genome comprises” the engineered gene. This indicates that the endogenous gene loci of the animal has been modified and substantially all cells contain such modification.

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from a polynucleotide selected from SEQ ID NOS 1-58 and 67-72, or homologs thereof, etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. A transgenic animal which comprises the functional disruption can also be referred to as a “knock-out” animal, since the biological activity of the gene, or a polypeptide encoded for by it, has been “knocked-out.” Knock-outs can be homozygous or heterozygous.

For creating functional disrupted genes, and other gene mutations, homologous recombination technology is of special interest since it allows specific regions of the genome to be targeted. Using homologous recombination methods, genes can be specifically-inactivated, specific mutations can be introduced, and exogenous sequences can be introduced at specific sites. These methods are well known in the art, e.g., as described in the patents above. See, also, Robertson, Biol. Reproduc., 44(2):238-245, 1991. Generally, the genetic engineering is performed in an embryonic stem (ES) cell, or other pluripotent cell line (e.g., adult stem cells, EG cells), and that genetically-modified cell (or nucleus) is used to create a whole organism. Nuclear transfer can be used in combination with homologous recombination technologies.

For example, the genes can be disrupted in mouse ES cells using a positive-negative selection method (e.g., Mansour et al., Nature, 336:348-352, 1988). In this method, a targeting vector can be constructed which comprises a part of the gene to be targeted. A selectable marker, such as neomycin resistance genes, can be inserted into an exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and germline animals, e.g., animals having somatic and/or germ cells comprising the engineered gene. Homozygous knock-out animals can be obtained from breeding heterozygous knock-out animals. See, e.g., U.S. Pat. No. 6,225,525.

A transgenic animal, or animal cell, lacking one or more functional genes represented by can be useful in a variety of applications, including, as an animal model for prostate diseases, for drug screening assays (e.g., for kinases or other functional classes), and any of the utilities mentioned in any issued U.S. patent on transgenic animals, including, U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824.

The present invention also relates to non-human, transgenic animal whose genome comprises a recombinant polynucleotide selected from SEQ ID NOS 1-58 and 67-72, or a homolog thereof, which is operatively linked to an expression control sequence effective to express said coding sequence, e.g., in prostate. Such a transgenic animal can also be referred to as a “knock-in” animal since an exogenous gene has been introduced, stably, into its genome.

A recombinant polynucleotide selected from SEQ ID NOS 1-58 and 67-72, or a homolog thereof, refers to a polynucleotide which has been introduced into a target host cell and optionally modified, such as cells derived from animals, plants, bacteria, yeast, etc. A recombinant polynucleotide includes completely synthetic nucleic acid sequences, semi-synthetic nucleic acid sequences, sequences derived from natural sources, and chimeras thereof. “Operable linkage” has the meaning used through the specification, i.e., placed in a functional relationship with another nucleic acid. When a gene is operably linked to an expression control sequence, as explained above, it indicates that the gene (e.g., coding sequence) is joined to the expression control sequence (e.g., promoter) in such a way that facilitates transcription and translation of the coding sequence. As described above, the phrase “genome” indicates that the genome of the cell has been modified. In this case, a polynucleotide selected from SEQ ID NOS 1-58 and 67-72, or a homolog thereof, has been stably integrated into the genome of the animal. A polynucleotide in operable linkage with the expression control sequence can also be referred to as a construct or transgene.

Any expression control sequence can be used depending on the purpose. For instance, if selective expression is desired, then expression control sequences which limit its expression can be selected. These include, e.g., tissue or cell-specific promoters, introns, enhancers, etc. For various methods of cell and tissue-specific expression, see, e.g., U.S. Pat. Nos. 6,215,040, 6,210,736, and 6,153,427. These also include the endogenous promoter, i.e., the coding sequence can be operably linked to its own promoter. Inducible and regulatable promoters can also be utilized.

The present invention also relates to a transgenic animal which contains a functionally disrupted and a transgene stably integrated into the animals genome. Such an animal can be constructed using combinations any of the above- and below-mentioned methods. Such animals have any of the aforementioned uses, including permitting the knock-out of the normal gene and its replacement with a mutated gene. Such a transgene can be integrated at the endogenous gene locus so that the functional disruption and “knock-in” are carried out in the same step.

In addition to the methods mentioned above, transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon etal., Proc. Natl. Acad. Sci., 77:7380-7384, 1980; Palmiteret al., Cell, 41:343-345, 1985; Paliniter et al., Ann. Rev. Genet., 20:465-499, 1986; Askew et al., Mol. Cell. Bio., 13:4115-4124, 1993; Games et al. Nature, 373:523-527, 1995; Valancius and Smithies, Mol. Cell. Bio., 11: 1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617,1993; Cibelli et al., Science, 280:1256-1258, 1998. For guidance on recombinase excision systems, see, e.g., U.S. Pat. Nos. 5,626,159, 5,527,695, and 5,434,066. See also, Orban, P. C., et al., “Tissue-and Site-Specific DNA Recombination in Transgenic Mice,” Proc. Natl. Acad. Sci. USA, 89:6861-6865 (1992); O'Gorman, S., et al., “Recombinase-Mediated Gene Activation and Site-Specific Integration in Mammalian Cells,” Science, 251:1351-1355 (1991); Sauer, B., et al., “Cre-stimulated recombination at 1oxP-Containing DNA sequences placed into the mammalian genome,” Polynucleotides Research, 17(1):147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest. 100:169-179; Barlow, C. et al. (1997) Nucl. Acids Res. 25:2543-2545; Araki, K. et al. (1997) Nucl. Acids Res. 25:868-872; Mortensen, R. N. et al. (1992) Mol. Cell. Biol. 12:2391-2395 (G418 escalation method); Lakhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

A polynucleotide according to the present invention can be introduced into any non-human animal, including a non-human mammal, mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-345, 1985), sheep (Hammer et al., Nature, 315:343-345, 1985), cattle, rat, or primate. See also, e.g., Church, 1987, Trends in Biotech. 5:13-19; Clark et al., Trends in Biotech. 5:20-24, 1987); and DePamphilis et al., BioTechniques, 6:662-680, 1988. Transgenic animals can be produced by the methods described in U.S. Pat. No. 5,994,618, and utilized for any of the utilities described therein.

Database

The present invention also relates to electronic forms of polynucleotides, polypeptides, etc., of the present invention, including computer-readable medium (e.g., magnetic, optical, etc., stored in any suitable format, such as flat files or hierarchical files) which comprise such sequences, or fragments thereof, e-commerce-related means, etc. Along these lines, the present invention relates to methods of retrieving gene sequences from a computer-readable medium, comprising, one or more of the following steps in any effective order, e.g., selecting a cell or gene expression profile, e.g., a profile that specifies that said gene is differentially expressed in prostate, and retrieving said differentially expressed gene sequences, where the gene sequences consist of the genes represented by SEQ ID NOS 1-58 and 67-72.

A “gene expression profile” means the list of tissues, cells, etc., in which a defined gene is expressed (i.e, transcribed and/or translated). A “cell expression profile” means the genes which are expressed in the particular cell type. The profile can be a list of the tissues in which the gene is expressed, but can include additional information as well, including level of expression (e.g., a quantity as compared or normalized to a control gene), and information on temporal (e.g., at what point in the cell-cycle or developmental program) and spatial expression. By the phrase “selecting a gene or cell expression profile,” it is meant that a user decides what type of gene or cell expression pattern he is interested in retrieving, e.g., he may require that the gene is differentially expressed in a tissue, or he may require that the gene is not expressed in blood, but must be expressed in prostate. Any pattern of expression preferences may be selected. The selecting can be performed by any effective method. In general, “selecting” refers to the process in which a user forms a query that is used to search a database of gene expression profiles. The step of retrieving involves searching for results in a database that correspond to the query set forth in the selecting step. Any suitable algorithm can be utilized to perform the search query, including algorithms that look for matches, or that perform optimization between query and data. The database is information that has been stored in an appropriate storage medium, having a suitable computer-readable format. Once results are retrieved, they can be displayed in any suitable format, such as HTML.

For instance, the user may be interested in identifying genes that are differentially expressed in a prostate. He may not care whether small amounts of expression occur in other tissues, as long as such genes are not expressed in peripheral blood lymphocytes. A query is formed by the user to retrieve the set of genes from the database having the desired gene or cell expression profile. Once the query is inputted into the system, a search algorithrn is used to interrogate the database, and retrieve results.

Advertising, Licensing, etc., Methods

The present invention also relates to methods of advertising, licensing, selling, purchasing, brokering, etc., genes, polynucleotides, specific-binding partners, antibodies, etc., of the present invention. Methods can comprises, e.g., displaying a SEQ ID NOS 1-58 and 67-72 gene, SEQ ID NOS 1-58 and 67-72 polypeptide, or antibody specific for SEQ ID NOS 1-58 and 67-72 in a printed or computer-readable medium (e.g., on the Web or Internet), accepting an offer to purchase said gene, polypeptide, or antibody.

Other

A polynucleotide, probe, polypeptide, antibody, specific-binding partner, etc., according to the present invention can be isolated. The term “isolated” means that the material is in a form in which it is not found in its original environment or in nature, e.g., more concentrated, more purified, separated from component, etc. An isolated polynucleotide includes, e.g., a polynucleotide having the sequenced separated from the chromosomal DNA found in a living animal, e.g., as the complete gene, a transcript, or a cDNA. This polynucleotide can be part of a vector or inserted into a chromosome (by specific gene-targeting or by random integration at a position other than its normal position) and still be isolated in that it is not in a form that is found in its natural environment. A polynucleotide, polypeptide, etc., of the present invention can also be substantially purified. By substantially purified, it is meant that polynucleotide or polypeptide is separated and is essentially free from other polynucleotides or polypeptides, i.e., the polynucleotide or polypeptide is the priinary and active constituent. A polynucleotide can also be a recombinant molecule. By “recombinant,” it is meant that the polynucleotide is an arrangement or form which does not occur in nature. For instance, a recombinant molecule comprising a promoter sequence would not encompass the naturally-occurring gene, but would include the promoter operably linked to a coding sequence not associated with it in nature, e.g., a reporter gene, or a truncation of the normal coding sequence.

The term “marker” is used herein to indicate a means for detecting or labeling a target. A marker can be a polynucleotide (usually referred to as a “probe”), polypeptide (e.g., an antibody conjugated to a detectable label), PNA, or any effective material.

The topic headings set forth above are meant as guidance where certain information can be found in the application, but are not intended to be the only source in the application where information on such topic can be found. Reference materials

For other aspects of the polynucleotides, reference is made to standard textbooks of molecular biology. See, e.g., Hames et al., Polynucleotide Hybridization IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the present invention to its fuillest extent. The following preferred specific embodiments are, therefore, to be construed as merely illustrative, and not limitative of the remainder of the disclosure in any way whatsoever. The entire disclosure of all applications, patents and publications, cited above and in the figures are hereby incorporated by reference in their entirety.

TABLE 1

| | Type of | Clone seq | Genomic Seq. |
|---|---|---|---|
| Clone name | Polymorphism | (Pos/nt) | (Accn#, nt) |
| PR104 | Substitution | 35, T | NT_005991, G |
| | Substitution | 861, T | NT_005991, C |
| PR302 | Substitution | 986, T | AL137878, C |
| | Substitution | 1040, T | AL137878, C |
| | Insertion | 1479, A | AL137878, * |
| | Substitution | 2081, C | AL137878, T |
| | Substitution | 2243, T | AL137878, A |
| | Substitution | 2305, A | AL137878, G |
| | Substitution | 2495, C | AL137878, T |
| | Insertion | 2789, T | AL137878, * |
| | Substitution | 3061, C | AL137878, T |
| PR371 | Substitution | 297, C | NT_022448, T |
| | Insertion | 905, A | NT_022448, * |
| | Substitution | 2190, G | NT_022448, A |
| | Deletion | 2226, *** | NT_022448, |
| | | | ACT |
| | Substitution | 2699, G | NT_022448, A |
| | Substitution | 3223, T | NT_022448, C |
| | Substitution | 5255, G | NT_022448, A |
| PR398 | Substitution | 190, T | AC005837, C |
| | Substitution | 201, A | AC005837, G |
| | Substitution | 202, A | AC005837, G |
| Pr333(BC1226G12) | Substitution | 2185, G | AL031431, A |
| | Substitution | 2217, T | AL031431, C |
| | Insertion | 2287, A | AL031431, * |
| | Substitution | 2367, G | AL031431, A |
| Pr421(AB1764A04) | Substitution | 121, A | AL1764A04, G |
| PR1234E11(PR410) | substitution | 274, C | NT_004836, T |
| | deletion | 732-733, * | NT_004836, T |
| | substitution | 767, T | NT_004836, C |
| | deletion | 933-934, *** | NT_004836, |
| | | | TTT |
| | substitution | 1222, G | NT_004836, A |
| | substitution | 1224, G | NT_004836, A |

1. An isolated polynucleotide comprising,
a polynucleotide sequence selected from SEQ ID NOS 1-58 and 67-72, or a complement thereto.

2. An isolated polynucleotide of claim 1 which codes without interruption for an amino acid sequence selected from SEQ ID NOS 1-58 and 67-72, or a complement thereto.

3. An isolated polynucleotide comprising,
a polynucleotide sequence having 90% or more sequence identity to a polynucleotide sequence selected from SEQ ID NOS 1-58 and 67-72 of claim 1 and which codes without interruption for a polypeptide sequence selected from SEQ ID NOS 1-58 and 67-72, or a complement thereto.

4. An isolated polynucleotide consisting of:
a polynucleotide fragment which is specific for a polynucleotide selected from SEQ ID NOS 1-58 and 67-72 of claim 1, or a complement thereof.

5. An isolated polypeptide comprising,
an amino acid sequence selected from SEQ ID NOS 1-58 and 67-72 of claim 1, or polypeptide fragment thereof which is specific-for a polypeptide selected from SEQ ID NOS 1-58 and 67-72.

6. An isolated polypeptide comprising an amino acid sequence having 90% or more sequence identity to an amino acid sequence selected from SEQ ID NOS 1-58 and 67-72 of claim 1.

7. A method of detecting a nucleic acid coding for a polypeptide selectively expressed in prostate, comprising,
contacting a sample comprising nucleic acid with a polynucleotide probe specific for a prostate selective polynucleotide of claim 1 under conditions effective for said probe to hybridize specifically with said nucleic acid, and
detecting hybridization between said probe and said nucleic acid.

8. A method of claim 7, wherein said detecting is performed by:
Northern blot analysis, polymerase chain reaction (PCR), reverse transcriptase PCR, RACE PCR, or in situ hybridization.

9. (canceled)

10. A method of diagnosing a prostate disease associated with abnormal expression of at least one polynucleotide or polypeptide selected from SEQ ID NOS 1-58 and 67-72 of claim 1, comprising:
assessing the expression of at least one polynucleotide or polypeptide selected from SEQ ID NOS 1-58 and 67-72 in a tissue sample comprising prostate cells, or cells derived from prostate.

11. A method of claim 10, wherein assessing is:
measuring expression levels of said polynucleotide, determining the genomic structure of said polynucleotide, determining the mRNA structure of transcripts from said gene, or measuring the expression levels of polypeptide coded for by said polynucleotide.

12. A method of claim 11, further comprising:
comparing said expression to the expression of a gene of a known normal tissue.

13. (canceled)

14. A method of assessing a therapeutic or preventative intervention in a subject having a prostate disease, comprising,
determining the expression levels of a polynucleotide or polypeptide selected from SEQ ID NOS 1-58 and 67-72 of claim 1 in a tissue sample comprising prostate cells, or cells derived from prostate.

15. A method for identifying an agent that modulates the expression of a polynucleotide or polypeptide selected from SEQ ID NOS 1-58 and 67-72 in prostate cells, cells derived from prostate, or prostate progenitor cells, comprising,
contacting a cell population with a test agent under conditions effective for said test agent to modulate the expression of a polynucleotide or polypeptide selected from SEQ ID NOS 1-58 and 67-72 of claim 1 in prostate cells, or prostate progenitor cells, and
determining whether said test agent modulates said a polynucleotide or polypeptide selected from SEQ ID NOS 1-58 and 67-72.

16. A method of claim 15, wherein said agent is an antisense polynucleotide to a target polynucleotide sequence selected from SEQ ID NOS 1-58 and 67-72 and which is effective to inhibit translation of said polynucleotide sequence.

17. A method of detecting polymorphisms in polynucleotide sequence selected from SEQ ID NOS 1-58 and 67-72 comprising:
comparing the structure of: genomic DNA comprising all or part of a polynucleotide sequence selected from SEQ ID NOS 1-58 and 67-72 of claim 1, mRNA comprising all or part of a polynucleotide sequence selected from SEQ ID NOS 1-58 and 67-72, or cDNA comprising all or part of a polynucleotide sequence selected from SEQ ID NOS 1-58 and 67-72, with the structure of polynucleotide or polypeptide sequence selected from SEQ ID NOS 1-58 and 67-72.

18. A method of claim 17, wherein said polymorphism is a nucleotide deletion, substitution, inversion, or transposition.

19. A non-human, transgenic mammal whose genome comprises a functional disruption of a polynucleotide sequence selected from SEQ ID NOS 1-58 and 67-72 of claim 1, or a homolog thereof.

20. A non-human, transgenic mammal of claim 19, whose genome further comprises said polynucleotide operatively linked to an expression control sequence effective to express said gene in prostate cells, cells derived from prostate, or prostate progenitor cells.

21. (canceled)

22. A mammalian cell whose genome comprises a functional disruption of a polynucleotide sequence selected from SEQ ID NOS 1-58 and 67-72 of claim 1, or a homolog thereof.

23. A mammalian cell of claim 22, wherein said cell is a prostate, cell derived from prostate, or a prostate progenitor cell.

24. (canceled)

25. (canceled)

26. A non-human, transgenic mammal of claim 20, whose genome further comprises a functional disruption of the endogenous polynucleotide sequence selected from SEQ ID NOS 1-58 and 67-72, or a homolog thereof.

27. A method of advertising a polynucleotide sequence selected from SEQ ID NOS 1-58 and 67-72, or a homolog thereof, for sale, commercial use, or licensing, comprising,
displaying in a computer-readable medium a polynucleotide selected from SEQ ID NOS 1-58 and 67-72 of claim 1, effective specific fragments thereof, or complements thereto.

28. An antibody which is specific-for a polypeptide selected from SEQ ID NOS 1-58 and 67-72.

29. A method for identifying prostate tissue or cells in a sample, comprising:
determining the number of target polynucleotides which are expressed in a sample, wherein said target polynucleotides comprise a polynucleotide of claim 1, whereby said number is indicative of the probability that said sample comprises prostate tissue or cells.


SEQUENCE LISTING (72 sequences)

SEQ ID NO: 1 - DNA, 2244 bp, Homo sapiens
  CDS (36)..(2033)
ggcagtgcggcgggttacggcctggtcagaccataatgacttcagcaaataaagcaatcgaattacaactacaagtgaaacaaaatgcagaagaattacaagactttatgcgggatttagaaaactgggaaaaagacattaaacaaaaggatatggaactaagaagacagaatggtgttcctgaagagaatttacctcctattcgaaatgggaattttaggaaaaagaagaaaggcaaagctaaagagtcttccaaaaaaaccagagaggaaaacacaaaaaacaggataaaatcttatgattatgaggcatgggcaaaacttgatgtggaccgtatccttgatgagcttgacaaagacgatagtacccatgagtctctgtctcaagaatcagagtcggaagaagatgggattcatgtagattcacaaaaggctcttgttttaaaagaaaagggcaataaatacttcaaacaaggaaaatatgatgaagcaattgactgctacacaaaaggcatggatgccgatccatataatcccgtgttgccaacgaacagagcgtcagcatattttagactgaaaaaatttgctgttgctgagtctgattgtaatttagcagttgccttgaatagaagttatacaaaggcttattccagacgaggtgctgctcgatttgctttgcaaaaattagaagaggccaaaaaagattatgaaagagtattagaactagaaccaaataactttgaagcaacaaatgaactcaggaaaatcagtcaggctttagcatccaaagaaaactcatatccaaaggaagctgacatagtgattaagtcaacagaaggagagcgaaagcaaattgaagcacaacagaataagcagcaggccatttcagagaaagatcgggggaatggatttttcaaagaggggaaatatgaaagagcaattgaatgctatactcgagggatagcagcagatggtgctaatgcccttcttccagctaacagagctatggcctatctgaagattcagaaatatgaagaagctgaaaaagactgcacacaagccattttattagatggctcatattctaaagcttttgccagaagaggaactgcaagaacatttttgggaaagctaaatgaggcaaaacaagattttgaaactgttttacttctggaacctggaaataagcaagcagtaactgaactctccaaaattaaaaaggaattaattgagaaaggacactgggatgatgtctttcttgattccacacaaagacaaaatgtggtaaaacccattgataatccaccgcatcctggatcaactaaaccactcaagaaggttattattgaagaaactggtaatttgatacagactattgatgtgccagatagcactactgctgctgctccagagaataatcctattaatctagcaaatgtaatagcagccacaggcaccacaagtaagaagaattcaagccaagatgacctttttcccacaagtgatactccaagagcaaaagtattgaaaatagaagaagtcagtgatacttcatccctgcaacctcaagccagtttgaagcaggatgtatgtcagtcttacagcgagaaaatgcccatagagatagaacaaaaacctgctcagtttgccacaactgttcttcctccaattcctgcaaactcgttccagctcgaatctgatttcagacaattgaaaagttctccagatatgttgtatcagtatttaaagcaaattgaaccatctttgtatcctaagttgtttcagaaaaatctggatccagatgtattcaaccagatcgttaaaattctgcatgacttttacattgagaaagaaaagccattactcatctttgaaatcttacaaagactttctgaactaaaaaggtttgatatggcagtgatgtttatgtcagaaacagagaaaaagattgcacgtgcattatttaatcacatagacaagtcaggattgaaggatagttctgtcgaagaactcaagaaaagatacggtggttgatttccatttttgctgaaataattgtttttgactttcatatgtaaattttttctactgaaagtgttttgctttttaagaaaatgaaattatatagcaggaaaggactatctttgaacataagttaattaactataaggtgaattgtgatttaactagtgagaattgtattcaagtgaactctgtttttctgaaaataaaaatataaacaatg

SEQ ID NO: 2 - protein, 665 aa, Homo sapiens
MTSANKAIELQLQVKQNAEELQDFMRDLENWEKDIKQKDMELRRQNGVPEENLPPIRNGNFRKKKKGKAKESSKKTREENTKNRIKSYDYEAWAKLDVDRILDELDKDDSTHESLSQESESEEDGIHVDSQKALVLKEKGNKYFKQGKYDEAIDCYTKGMDADPYNPVLPTNRASAYFRLKKFAVAESDCNLAVALNRSYTKAYSRRGAARFALQKLEEAKKDYERVLELEPNNFEATNELRKISQALASKENSYPKEADIVIKSTEGERKQIEAQQNKQQAISEKDRGNGFFKEGKYERAIECYTRGIAADGANALLPANRAMAYLKIQKYEEAEKDCTQAILLDGSYSKAFARRGTARTFLGKLNEAKQDFETVLLLEPGNKQAVTELSKIKKELIEKGHWDDVFLDSTQRQNVVKPIDNPPHPGSTKPLKKVIIEETGNLIQTIDVPDSTTAAAPENNPINLANVIAATGTTSKKNSSQDDLFPTSDTPRAKVLKIEEVSDTSSLQPQASLKQDVCQSYSEKMPIEIEQKPAQFATTVLPPIPANSFQLESDFRQLKSSPDMLYQYLKQIEPSLYPKLFQKNLDPDVFNQIVKILHDFYIEKEKPLLIFEILQRLSELKRFDMAVMFMSETEKKIARALFNHIDKSGLKDSSVEELKKRYGG

SEQ ID NO: 3 - DNA, 5921 bp, Homo sapiens
  CDS (472)..(3591)
aaataccactcactatagggcggccgcgaattcggcaccagagtctcgagttttttttttttttttttttttttgagagggagtcttgctctgtcacccaggttggagtgcagtggcgcgatctcagctcactgcaacctccacctcctgggttcaggccattctcttgcctcagcctccagagtagctgggattacaggtgcctgccaacatgcccagctgagttttgtattttagcagagatggggtttcaccatgttgcctagcctggtcttgaaatcctgacctcaagttatccgcccacctcactctcccaaagtgctgagattacaggcgtgagccaccgcacccgtccacatcaaactagtttctacacaaccaagatgatgaaaacagtctgaaaataagacttgaaggtatcctttgtgtgtctatttaagctaacaataccagtttccataagcaggctcaatgaaatccttccttcctgtccacaccatcgtgcttatcagggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggcctgatgaagtacatcggggaggtggtgagagataacaccatcagcaggagttcagaggagaatattgtggccattggcatagcagcttggggcatggtctccaaccgggacaccctcatcaggaattgcgatgctgagggctattttttagcccagtaccttatggatgacttcacaagagatccactgtatatcctggacaacaaccacacacatttgctgctcgtggacaatggctgtcatggacatcccactgtcgaagcaaagctccggaatcagctagagaagtatatctctgagcgcactattcaagattccaactatggtggcaagatccccattgtgtgttttgcccaaggaggtggaaaagagactttgaaagccatcaatacctccatcaaaaataaaattccttgtgtggtggtggaaggctcgggccagatcgctgatgtgatcgctagcctggtggaggtggaggatgccctgacatcttctgccgtcaaggagaagctggtgcgctttttaccccgcacggtgtcccggctgcctgaggaggagactgagagttggatcaaatggctcaaagaaattctcgaatgttctcacctattaacagttattaaaatggaagaagctggggatgaaattgtgagcaatgccatctcctacgctctatacaaagccttcagcaccagtgagcaagacaaggataactggaatgggcagctgaagcttctgctggagtggaaccagctggacttagccaatgatgagattttcaccaatgaccgccgatgggagtctgctgaccttcaagaagtcatgtttacggctctcataaaggacagacccaagtttgtccgcctctttctggagaatggcttgaacctacggaagtttctcacccatgatgtcctcactgaactcttctccaaccacttcagcacgcttgtgtaccggaatctgcagatcgccaagaattcctataatgatgccctcctcacgtttgtctggaaactggttgcgaacttccgaagaggcttccggaaggaagacagaaatggccgggacgagatggacatagaactccacgacgtgtctcctattactcggcaccccctgcaagctctcttcatctgggccattcttcagaataagaaggaactctccaaagtcatttgggagcagaccaggggctgcactctggcagccctgggagccagcaagcttctgaagactctggccaaagtgaagaacgacatcaatgctgctggggagtccgaggagctggctaatgagtacgagacccgggctgttgagctgttcactgagtgttacagcagcgatgaagacttggcagaacagctgctggtctattcctgtgaagcttggggtggaagcaactgtctggagctggcggtggaggccacagaccagcatttcatcgcccagcctggggtccagaattttctttctaagcaatggtatggagagatttcccgagacaccaagaactggaagattatcctgtgtctgtttattatacccttggtgggctgtggctttgtatcatttaggaagaaacctgtcgacaagcacaagaagctgctttggtactatgtggcgttcttcacctcccccttcgtggtcttctcctggaatgtggtcttctacatcgccttcctcctgctgtttgcctacgtgctgctcatggatttccattcggtgccacacccccccgagctggtcctgtactcgctggtctttgtcctcttctgtgatgaagtgagacagtggtacgtaaatggggtgaattattttactgacctgtggaatgtgatggacacgctggggcttttttacttcatagcaggaattgtatttcggctccactcttctaataaaagctctttgtattctggacgagtcattttctgtctggactacattattttcactctaagattgatccacatttttactgtaagcagaaacttaggacccaagattataatgctgcagaggatgctgatcgatgtgttcttcttcctgttcctctttgcggtgtggatggtggcctttggcgtggccaggcaagggatccttaggcagaatgagcagcgctggaggtggatattccgttcggtcatctacgagccctacctggccatgttcggccaggtgcccagtgacgtggatggtaccacgtatgactttgcccactgcaccttcactgggaatgagtccaagccactgtgtgtggagctggatgagcacaacctgccccggttccccgagtggatcaccatccccctggtgtgcatctacatgttatccaccaacatcctgctggtcaacctgctggtcgccatgtttggctacacggtgggcaccgtccaggagaacaatgaccaggtctggaagttccagaggtacttcctggtgcaggagtactgcagccgcctcaatatccccttccccttcatcgtcttcgcttacttctacatggtggtgaagaagtgcttcaagtgttgctgcaaggagaaaaacatggagtcttctgtctgctgtttcaaaaatgaagacaatgagactctggcatgggagggtgtcatgaaggaaaactaccttgtcaagatcaacacaaaagccaacgacacctcagaggaaatgaggcatcgatttagacaactggatacaaaagcttaatgatctcaagggtcttctgaaagagattgctaataaaatcaaataaaactgtatgaactctaatggagaaaaatctaattatagcaagatcatattaaggaatgctgatgaacaattttgctatcgactactaaatgagagattttcagacccctgggtacatggtggatgattttaaatcaccctagtgtgctgagaccttgagaataaagtgtgtgattggtttcatacttgaagacggatataaaggaagaatatttcctttatgtgtttctccagaatggtgcctgtttctctctgtgtctcaatgcctgggactggaggttgatagtttaagtgtgttcttaccgcctcctttttcctttaatcttatttttgatgaacacatatataggagaacatctatcctatgaataagaacctggtcatgctttactcctgtattgttattttgttcatttccaattgattctctacttttcccttttttgtattatgtgactaattagttggcatattgttaaaagtctctcaaattaggccagattctaaaacatgctgcagcaagaggaccccgctctcttcaggaaaagtgttttcatttctcaggatgcttcttacctgtcagaggaggtgacaaggcagtctcttgctctcttggactcaccaggctcctattgaaggaaccacccccattcctaaatatgtgaaaagtcgcccaaaatgcaaccttgaaaggcactactgactttgttcttattggatactcctcttatttattatttttccattaaaaataatagctggctattatagaaaatttagaccatacagagatgtagaaagaacataaattgtccccattaccttaaggtaatcactgctaacaatttctggatggtttttcaagtctattttttttctatgtatgtctcaattctctttcaaaattttacagaatgttatcatactacatatatactttttatgtaagctttttcacttagtattttatcaaatatgtttttattatattcatagccttcttaaacattatatcaataattgcataataggcaacctctagcgattaccataattttgctcattgaaggctatctccagttgatcattgggatgagcatctttgtgcatgaatcctattgctgtatttgggaaaattttccaaggttagattccaataaatatctatttattattaaatattaaaatatctatttattattaaaaccatttataaggctttttcataaatgtatagcaaataggaattattaacttgagcataagatatgagatacatgaacctgaactattaaaataaaatattatatttaacccttagtttaagaagaagtcaatatgcttatttaaatattatggatggtgggcagatcacttgaggtcaggagttcgagaccagcctggccaacatggcaaaaccacatctctactaaaaataaaaaaattagctgggtgtggtggtgcactcctgtaatcccagctactcagaaggctgaggtacaagaattgctggaacctgggaggcggaggttgcagtgaaccaagattgcaccactgcactccagccggggtgacagagtgagactccgactgaaaataaataaataaataaataaataaataaataaataaatattatggatggtgaagggaatggtatagaattggagagattatcttactgaacacctgtagtcccagctttctctggaagtggtcgtatttgagcaggatgtgcacaaggcaattgaaatgcccataattagtttctcagctttgaatacactataaactcactggctgaaggaggaaattttagaaggaagctactaaaagatctaatttgaaaaactacaaaagcattaactaaaaaagtttattttccttttgtctgggcagtagtgaaaataactactcacaacattcactatgtttgcaaggaattaacacaaataaaagatgcctttttacttaaacaccaagacagaaaacttgcccaatactgagaagcaacttgcattagagagggaactgttaaatgttttcaacccagttcatctggtggatgtttttgcaggttactctgagaattttgcttatgaaaaatcattatttttagtgtagttcacaataatgtattgaacatacttctaatcaaaggtgctatgtccttgtgtatggtactaaatgtgtcctgtgtacttttgcacaactgagaatcctgcagcttggtttaatgagtgtgttcatgaaataaataatggaggaataaaaaaaaaaaaaaa

SEQ ID NO: 4 - protein, 1039 aa, Homo sapiens
MKSFLPVHTIVLIRENVCKCGYAQSQHMEGTQINQSEKWNYKKHTKEFPTDAFGDIQFETLGKKGKYIRLSCDTDAEILYELLTQHWHLKTPNLVISVTGGAKNFALKPRMRKIFSRLIYIAQSKGAWILTGGTHYGLMKYIGEVVRDNTISRSSEENIVAIGIAAWGMVSNRDTLIRNCDAEGYFLAQYLMDDFTRDPLYILDNNHTHLLLVDNGCHGHPTVEAKLRNQLEKYISERTIQDSNYGGKIPIVCFAQGGGKETLKAINTSIKNKIPCVVVEGSGQIADVIASLVEVEDALTSSAVKEKLVRFLPRTVSRLPEEETESWIKWLKEILECSHLLTVIKMEEAGDEIVSNAISYALYKAFSTSEQDKDNWNGQLKLLLEWNQLDLANDEIFTNDRRWESADLQEVMFTALIKDRPKFVRLFLENGLNLRKFLTHDVLTELFSNHFSTLVYRNLQIAKNSYNDALLTFVWKLVANFRRGFRKEDRNGRDEMDIELHDVSPITRHPLQALFIWAILQNKKELSKVIWEQTRGCTLAALGASKLLKTLAKVKNDINAAGESEELANEYETRAVELFTECYSSDEDLAEQLLVYSCEAWGGSNCLELAVEATDQHFIAQPGVQNFLSKQWYGEISRDTKNWKIILCLFIIPLVGCGFVSFRKKPVDKHKKLLWYYVAFFTSPFVVFSWNVVFYIAFLLLFAYVLLMDFHSVPHPPELVLYSLVFVLFCDEVRQWYVNGVNYFTDLWNVMDTLGLFYFIAGIVFRLHSSNKSSLYSGRVIFCLDYIIFTLRLIHIFTVSRNLGPKIIMLQRMLIDVFFFLFLFAVWMVAFGVARQGILRQNEQRWRWIFRSVIYEPYLAMFGQVPSDVDGTTYDFAHCTFTGNESKPLCVELDEHNLPRFPEWITIPLVCIYMLSTNILLVNLLVAMFGYTVGTVQENNDQVWKFQRYFLVQEYCSRLNIPFPFIVFAYFYMVVKKCFKCCCKEKNMESSVCCFKNEDNETLAWEGVMKENYLVKINTKANDTSEEMRHRFRQLDTKA

SEQ ID NO: 5 - DNA, 1735 bp, Homo sapiens
  CDS (94)..(1296)
gtcgggacccagccagccctgcgcctcgcgccgtcgcgcatgcgtcctggtctttctctagagttgtatatatagaacatcctggagtccaccatgaacggacagttggatctaagtgggaagctaatcatcaaagctcaacttggggaggatattcggcgaattcctattcataatgaagatattacttatgatgaattagtgctaatgatgcaacgagttttcagaggaaaacttctgagtaatgatgaagtaacaataaagtataaagatgaagatggagatcttataacaatttttgatagttctgacctttcctttgcaattcagtgcagtaggatactgaaactgacattatttgttaatggccagccaagaccccttgaatcaagtcaggtgaaatatctccgtcgagaactgatagaacttcgaaataaagtgaatcgtttattggatagcttggaaccacctggagaaccaggaccttccaccaatattcctgaaaatgatactgtggatggtagggaagaaaagtctgcttctgattcttctggaaaacagtctactcaggttatggcagcaagtatgtctgcttttgatcctttaaaaaaccaagatgaaatcaataaaaatgttatgtcagcgtttggcttaacagatgatcaggtttcagggccacccagtgctcctgcagaagatcgttcaggaacacccgacagcattgcttcctcctcctcagcagctcacccaccaggcgttcagccacagcagccaccatatacaggagctcagactcaagcaggtcagattgaaggtcagatgtaccaacagtaccagcaacaggccggctatggtgcacagcagccgcaggctccacctcagcagcctcaacagtatggtattcagtattcagcaagctatagtcagcagactggacctcaacaacctcagcagttccagggatatggccagcaaccaacttcccaggcaccagctcctgccttttctggtcagcctcaacaactgcctgctcagccgccacagcagtaccaggcgagcaattatcctgcacaaacttacactgcccaaacttctcagcctactaattatactgtggctcctgcctctcaacctggaatggctccaagccaacctggggcctatcaaccaagaccaggttttacttcacttcctggaagtaccatgacccctcctccaagtgggcctaatccttatgcgcgtaaccgtcctccctttggtcagggctatacccaacctggacctggttatcgataaggaggctcctctacaccaattaatgtagctgctagctattggcctcccaaaagactccagtactattttaatttgtattgaagaagttcagaaatttaaaagcagagcattttttatgatatcattgttggtgttaattgaaagtataatttgctggaacacaaagaccaaaatgaaagttttttcctccctgcttaaaaatgtagcagcttcttagttactttggaacactactcttacatgtataaagtgattgacttgactttctagcttcccttgtccggaggatattaaaatgctagggtgaggtttagccatcttacttggctttttactattaacatgatgtactaaagtagagccctttgagaatacaagatattatgtataaaatgtaacactgatgataggttaataaagatgattgaatccattagtg

SEQ ID NO: 6 - protein, 400 aa, Homo sapiens
MNGQLDLSGKLIIKAQLGEDIRRIPIHNEDITYDELVLMMQRVFRGKLLSNDEVTIKYKDEDGDLITIFDSSDLSFAIQCSRILKLTLFVNGQPRPLESSQVKYLRRELIELRNKVNRLLDSLEPPGEPGPSTNIPENDTVDGREEKSASDSSGKQSTQVMAASMSAFDPLKNQDEINKNVMSAFGLTDDQVSGPPSAPAEDRSGTPDSIASSSSAAHPPGVQPQQPPYTGAQTQAGQIEGQMYQQYQQQAGYGAQQPQAPPQQPQQYGIQYSASYSQQTGPQQPQQFQGYGQQPTSQAPAPAFSGQPQQLPAQPPQQYQASNYPAQTYTAQTSQPTNYTVAPASQPGMAPSQPGAYQPRPGFTSLPGSTMTPPPSGPNPYARNRPPFGQGYTQPGPGYR

SEQ ID NO: 7 - DNA, 1332 bp, Homo sapiens
  CDS (97)..(897)
gcgagccgggctgtcgggtgtgttttgctctccatcctccgtcgtctctgcagcactccgggttctcctccagagcgctagtcccaggagctcggaatgttcgtggaacttaataacctgcttaacaccacccccgacagggcggagcaggggaaactgactctactctgtgatgccaagacagatgggagtttccttgtacaccactttctctccttctatctcaaagctaattgtaaagtctgctttgtggcactcatccagtccttcagccactacagtatcgtgggacagaagctgggtgtcagcctgaccatggcgcgggagcgtgggcagcttgtgttccttgagggactcaagtctgcagtggacgtcgtcttccaggctcaaaaggagccacaccccctgcagtttctcagggaggctaatgctgggaacttgaaaccattgtttgagtttgtacgggaggccctgaagccagtagacagtggagaggctcggtggacgtacccggtgctgttggtggacgacctcagtgtgctcctgagcctgggcatgggggcggtggctgtgctagacttcattcactactgcagagccaccgtgtgctgggaactaaagggaaacatggtggtccttgtgcacgacagtggagatgcggaggatgaggagaatgacatcctgctgaatggcctcagtcatcagagccatctgatactgcgggctgagggcctggccactggcttctgcagggatgtgcacgggcagctgaggatcctgtggaggagaccatcgcagcccgcagtccaccgggatcagagcttcacttaccagtataagatacaggacaaaagcgtgtctttttttgccaaaggaatgtctcctgctgttctgtgacctgatttcggagcagctgaagctacataggactgtttttggacgtggaagatagagcaacatagcaagaatgggtctttctcctctgtagtaatatttcaggctggaccggcgactccactgtgaccagagggttgagtgctgcagtgatggcatgccttggctgccctgggccctgttcagaaaacacaagggaccacaatcctgcctttgctgagagagaggctggatgctagacccaagtgaaaggggtcctttggagcctttgtttaaatatgccttagccccagctgcccatttttggttgacaagcctttcagagccagagtgggtatagatgtgccagccaggagatggcaccggatggcaggtgtgcaaggtgacaactaggataatcatggctggaataaagtaagtttccacactggaaaaaaa

SEQ ID NO: 8 - protein, 266 aa, Homo sapiens
MFVELNNLLNTTPDRAEQGKLTLLCDAKTDGSFLVHHFLSFYLKANCKVCFVALIQSFSHYSIVGQKLGVSLTMARERGQLVFLEGLKSAVDVVFQAQKEPHPLQFLREANAGNLKPLFEFVREALKPVDSGEARWTYPVLLVDDLSVLLSLGMGAVAVLDFIHYCRATVCWELKGNMVVLVHDSGDAEDEENDILLNGLSHQSHLILRAEGLATGFCRDVHGQLRILWRRPSQPAVHRDQSFTYQYKIQDKSVSFFAKGMSPAVL

SEQ ID NO: 9 - DNA, 2559 bp, Homo sapiens
  CDS (249)..(1742)
ccgtatttcgcagattggagctgagctgtggctgccagaagatagcgaacgaatggaaactaaaagtggaaatcaggaaaaggtaatggaagaagaaagcactgaaaagaaaaaagaagttgaaaaaaagaaacggtcacgagttaaacaggtgcttgcagatattgctaagcaagtggacttctggtttggggatgcaaatcttcacaaggatagatttcttcgagaacagatagaaaaatctagagatggatatgttgatatcactacttgtgtcttttaacaaaatgaaaaaattgactactgatgggaagttaattgccagagcattgagaagttcagctgttgtagagcttgatttggaaggcaccagaatccggaggaaaaaacctctgggggaaagaccaaaggatgaggatgaacgcacagtgtatgtggagttacttcccaaaaatgttaatcacagctggattgaaagagtatttgggaaatgtggcaatgttgtttatataagtataccacattataagtctactggagatccaaagggatttgcgtttgtggaatttgaaacaaaagaacaagcagcaaaagcaattgagtttcttaacaacccaccagaagaagcaccaagaaaacctggcatatttcctaaaacagtgaaaaataagcccattccagccttaagagttgtggagaagaaaaagaaaaagaagaagaaaggccgaatgaaaaaggaagacaatatccaagccaaagaagaaaacatggacacaagcaacaccagcatcagtaaaatgaaaagatccagacccacatctgagggctctgacattgagtccactgaaccccaaaagcagtgctcaaagaaaaagaaaaaacaggacagagttgaagcatctagcttacctgaagtcagaacagggaagaggaagagaagcagctctgaagatgcagaatccctagctccccgatcaaaagtaaagaaaattattcagaaagacatcattaaggaagcatcagaagcttccaaggaaaatagagatatagaaatctctactgaagaggaaaaggatactggagatctaaaagatagctctctcttgaaaacaaaaaggaaacataagaaaaaacataaagagagacataaaatgggagaagaagttataccattaagagtgctatcaaagagcgaatggatggatttgaaaaaagagtatttagcgctacaaaaagctagcatggcttctttaaaaaaaacaatatcccaaataaaatcagagtcagaaatggaaacagacagtggagtacctcaaaacactggaatgaaaaatgaaaaaacagccaacagggaagagtgtcgcacccaggagaaagttaatgcaacaggaccacagttcgtgagtggagtgattgtgaagatcattagcacagagcctctacctggcaggaaacaagtccgggatactttggcagcaatctcagaagttctttatgttgatttgctagaaggggatacagaatgccatgctagatttaaaactcctgaggatgctcaagcagtaataaatgcctatacagaaattaacaagaaacactgctggaaactcgagatcctttctggtgatcacgaacaaaggtattggcagaagattttggttgatagacaggcaaaacttaatcagcctcgggaaaagaaaagaggcactgaaaaggtaattgattcatttttgtttttttagactaaactttccttgaacgtttaatgccaaagtcagtactaggtagtcataaaatgatcgtagttttgctattacctaacaaaaatttactgagtatatactatgttttaggttgggagggaaaagtcatttctctagatttcaaaatgtatgaaattgaattatttttctcacaaggatacataattttaatttagattttttctttttaaccagtgatatactttaacagtcttgatatctggctgctatgtcttgtagtttggtgataaatgattatagaaagattatcttcatggtaaaaacacaatgtattcagaatgtagtttataaagataggaggctccatagataagatggagagactttatgcatagtgtttcctatagcaacgataagctttattcctctttctctaagatagtaggtaatacaaacaagcttaaataatttaaagcaagcccctctttcagtaattttatttttctgtcacttaaaatgatactggtctgatcagtagtggaatcacacctgggaatagccactgctttccagcttaggcaacatagcaagaccctgtctcttaaagttttaaaacaaattattatacatgtaaaggttagtacattttaaaagtgcctggaacaaagtagggtacataatacctaatattaactacagtcatcaaaatttagaaatgcatattataaaagtgtaagaaaaatgaaaaagatgaacttccttttttgttaggtgagcaagttaaaagtcctatttcctgtaatctaagcttgttgatttggggtcatatttatac

SEQ ID NO: 10 - protein, 497 aa, Homo sapiens
MDMLISLLVSFNKMKKLTTDGKLIARALRSSAVVELDLEGTRIRRKKPLGERPKDEDERTVYVELLPKNVNHSWIERVFGKCGNVVYISIPHYKSTGDPKGFAFVEFETKEQAAKAIEFLNNPPEEAPRKPGIFPKTVKNKPIPALRVVEKKKKKKKKGRMKKEDNIQAKEENMDTSNTSISKMKRSRPTSEGSDIESTEPQKQCSKKKKKQDRVEASSLPEVRTGKRKRSSSEDAESLAPRSKVKKIIQKDIIKEASEASKENRDIEISTEEEKDTGDLKDSSLLKTKRKHKKKHKERHKMGEEVIPLRVLSKSEWMDLKKEYLALQKASMASLKKTISQIKSESEMETDSGVPQNTGMKNEKTANREECRTQEKVNATGPQFVSGVIVKIISTEPLPGRKQVRDTLAAISEVLYVDLLEGDTECHARFKTPEDAQAVINAYTEINKKHCWKLEILSGDHEQRYWQKILVDRQAKLNQPREKKRGTEKVIDSFLFF

SEQ ID NO: 11 - DNA, 1859 bp, Homo sapiens
  CDS (212)..(1267)
ggcgaggggcctacgctgcggcccggcaacaaggcccgactcggcccctcgggaccagagccccacccgatcggaagcggatcctttaccagggccataggccagtgactaggccgggcctgggcctcccatcggggccggactaggacgaggccccggggaggcccctggcctaccagacccttttctcaggccgacagccgccaggaagatgcaacgtgccctgccaggcgcccgccagcacttgggggccattctggccagcgccagcgtggtggtgaaggctctgtgtgcggcggtactattcctctacctgctctccttcgccgtggacacaggctgcctggcggtcaccccgggctacctctttcctcccaacttctggatctggaccctggccacccatgggctgatggagcagcatgtgtgggacgtggccatcagcctgacaacggtggtggtggccgggcgtttgctggagcccctctggggggccttggagctgctcatcttcttctcagtggtgaatgtgtctgtagggctgctgggggccttcgcctacctcctcacctacatggcttccttcaacctggtctacctgttcactgtccgtatccacggcgccttgggcttcctaggtggcgtcctggtggcactcaagcaaaccatgggggactgtgtggtcctgcgagtgccccaggtgcgcgtcagtgtgatgcccatgctgctgctggcgctgctgctcctgctgcggctcgccacgctgctccagagcccggcgctggcttcctatggcttcgggctgctctccagttgggtatatcttcgcttctaccagcgccatagccggggccgaggggacatggctgaccactttgctttcgccactttcttccctgagatcctgcagcctgtggtgggtttgctggcgaacttggtgcacagcctcctggtgaaggtaaagatatgccagaagacggtgaagcgctacgatgtgggtgccccatcctccatcaccatcagcctgccaggcacagaccctcaagacgccgagcggagaaggcaactggccctgaaggcactcaatgagcggctgaagagagtggaagaccagtccatctggcccagcatggatgatgatgaagaggagtctggggccaaggtggacagccccctgccctcagacaaagctcccacacccccagggaagggggctgccccagaatccagtctaatcaccttcgaggcagctcccccgacgctgtaactccagaccaccttgagtgtggcacctcccctcccaagccccccttgacatcctctcagctactccagggcacctgactgctctgaggagagggaagaaggcctgctggggctttccatggccttctgctgtttctcgccaacactacccaggactcttgctacctggttccaactccagacaaccactatgccaggcccggagcctctgaggcatcggccagtccaggccctcatctgaggtaagaatgtacatcagctggcagccccaagcaagtggctgcagggacactgatgccacagctcctgggccggccctcacatctgaaactggttgccgagagccctgagccaaggcaaggatttgccaaaaatgttctgggggcccagcaaatgcaggagccgacctggggctgcacatccctgcccatccccagaaagactgttcctgtcaggatttgtttccctctgctgtggcggtgactgcttctggaccagaacagctccagctcccaggtattttctacaggaccacttgagtgggcagccaagcccaggctcgcagtatcaataaagcagttctctgaggaatg

SEQ ID NO: 12 - protein, 351 aa, Homo sapiens
MQRALPGARQHLGAILASASVVVKALCAAVLFLYLLSFAVDTGCLAVTPGYLFPPNFWIWTLATHGLMEQHVWDVAISLTTVVVAGRLLEPLWGALELLIFFSVVNVSVGLLGAFAYLLTYMASFNLVYLFTVRIHGALGFLGGVLVALKQTMGDCVVLRVPQVRVSVMPMLLLALLLLLRLATLLQSPALASYGFGLLSSWVYLRFYQRHSRGRGDMADHFAFATFFPEILQPVVGLLANLVHSLLVKVKICQKTVKRYDVGAPSSITISLPGTDPQDAERRRQLALKALNERLKRVEDQSIWPSMDDDEEESGAKVDSPLPSDKAPTPPGKGAAPESSLITFEAAPPTL

SEQ ID NO: 13 - DNA, 3494 bp, Homo sapiens
  CDS (608)..(1300)
gagctgaagcaggcgctcttggctcggcgcggcccgctgcaatccgtggaggaacgcgccgccgagccaccatcatgcctgggcacttacaggaaggttcggctgcgtggtcaccaaccgattcgaccagttatttgacgacgaatcggaccccttcgaggtgctgaaggcagcagagaacaagaaaaaagaagccggcgggggcggcgttgggggccctggggccaagagcgcagctcaggccgcggcccagaccaactccaacgcggcaggcaaacagctgcgcaaggagtcccagaaagaccgcaagaacccgctgccccccagcgttggcgtggttgacaagaaagaggagacgcagccgcccgtggcgcttaagaaagaaggaataagacgagttggaagaagacctgatcaacaacttcagggtgaagggaaaataattgatagaagaccagaaaggcgaccacctcgtgaacgaagattcgaaaagccacttgaagaaaagggtgaaggaggcgaattttcagttgatagaccgattattgaccgacctattcgaggtcgtggtggtcttggaagaggtcgagggggccgtggacgtggaatgggccgaggagatggatttgattctcgtggcaaacgtgaatttgataggcatagtggaagtgatagatcttctttttcacattacagtggcctgaagcacgaggacaaacgtggaggtagcggatctcacaactggggaactgtcaaagacgaattaacagagtcccccaaatacattcagaaacaaatatcttataattacagtgacttggatcaatcaaatgtgactgaggaaacacctgaaggtgaagaacatcatccagtggcagacactgaaaataaggagaatgaagttgaagaggtaaaagaggagggtccaaaagagatgactttggatgagtggaaggctattcaaaataaggaccgggcaaaagtagaatttaatatccgaaaaccaaatgaaggtgctgatgggcagtggaagaagggatttgttcttcataaatcaaagagtgaagaggctcatgctgaagattcggttatggaccatcatttccggaagccagcaaatgatataacgtctcagctggagatcaattttggagaccttggccgcccaggacgtggcggcaggggaggacgaggtggacgtgggcgtggtgggcgcccaaaccgtggcagcaggaccgacaagtcaagtgcttctgctcctgatgtggatgacccagaggcattcccagctctggcttaactggatgccataagacaaccctggttcctttgtgaacccttctgttcaaagcttttgcatgcttaaggattccaaacgactaagaaattaaaaaaaaaaagactgtcattcataccattcacacctaaagactgaattttatctgttttaaaaatgaacttctcccgctacacagaagtaacaaatatggtagtcagttttgtatttagaaatgtattggtagcagggatgttttcataattttcagagattatgcattcttcatgaatacttttgtattgctgcttgcaaatatgcatttccaaacttgaaatataggtgtgaacagtgtgtaccagtttaaagctttcacttcatttgtgttttttaattaaggatttagaagttcccccaattacaaactggttttaaatattggacatactggttttaatacctgctttgcatattcacacatggtcaactgggacatgttaaactttgatttgtcaaattttatgctgtgtggaatactaactatatgtattttaacttagttttaatattttcatttttggggaaaaatcttttttcacttctcatgatagctgttatatatatatgctaaatctttatatacagaaatatcagtacttgaacaaattcaaagcacatttggtttattaacccttgctccttgcatggctcattaggttcaaattataactgatttacattttcagctatatttactttttaaatgcttgagtttcccattttaaaatctaaactagacatcttaattggtgaaagttgtttaaactacttattgttggtaggcacatcgtgtcaagtgaagtagttttataggtatgggttttttctcccccttcaccagggtgggtggaataagttgatttggccaatgtgtaatatttaaactgttctgtaaaataagtgtctggccatttggtatgatttctgtgtgtgaaaggtcccaaaatcaaaatggtacatccataatcagccaccatttaacccttccttgttctaaaacaaaaaccaaagggcgctggttggtagggtgaggtgggggagtattttaatttttggaatttgggaagcagacagctttactttgtaaggttggaacagcagcactatacatgaaatataaaccaaaaacctttactgtttctaaatttcctagattgctattatttggttgtaagttgagtattccacagaaagtggtaattatctcttctctcttcctccattagaaaattaggtaaataatggattcctataatgggagcatcaccacttattaaaacacacatagaatgatgaattaaaaaagttttctaggattgtcttttattctgccacatttattgataaacagtgaaggaatttttaaaaaatttttaagaattgtttgtcacgtcatttttagaaatgttctacctgtatatggtaatgtccagttttaaaaatattggacatcttcaatcttaaacatttctatttagctgattggttctcacatatacttctaaaagaaacttttatgttataagagttactttttggataagatttattaatctcagttacctactattctgacattttaggaaggaggtaattgtttttaatgatggataaacttgtgctggtgttttggatcttatgatgctgagcatgttctgcactggtgctaatgtctaatataattttatatttacacacatacgtgctacccagagattaatttagtccatatgaactattgacccattgttcattgagacagcaacatacgcactcctaaatcagtgtgtttagacttttcaagtatctaactcatttccaaacatgtaccatgttttataaacctcttgatttccagcaacatactatagaaaacacctgctactcaaaacacaacttctcagtgtcatccattgctgtcgtgagagacaacatagcaatatctggtatgttgcaagctttcaagatagcctgaacttaaaaagttggtgcattagttgtatctgatggatataaatttgcctcctagttcactttgtgtcaagagctaaaactgtgaacctaactttctcttattggtgggtaataactgaaaataaagatttattttcatgctcacttcttaaaagtcataaaaacaatcaaaaaaaaaaaaaaa

SEQ ID NO: 14 - protein, 230 aa, Homo sapiens
MGRGDGFDSRGKREFDRHSGSDRSSFSHYSGLKHEDKRGGSGSHNWGTVKDELTESPKYIQKQISYNYSDLDQSNVTEETPEGEEHHPVADTENKENEVEEVKEEGPKEMTLDEWKAIQNKDRAKVEFNIRKPNEGADGQWKKGFVLHKSKSEEAHAEDSVMDHHFRKPANDITSQLEINFGDLGRPGRGGRGGRGGRGRGGRPNRGSRTDKSSASAPDVDDPEAFPALA

SEQ ID NO: 15 - DNA, 7921 bp, Homo sapiens
  CDS (92)..(2776)
cgccttgtgtgtgctggatcctgcgcgggtagatccccgagtaattttttctgcaggatgaattaagagaagagacacttgctcatcaggcatggagagcactttgtcagcttccaatatgcaagacccttcatcttcacccttggaaaagtgtctcggctcagctaatggaaatggagaccttgattctgaagaaggctcaagcttggaggaaactggctttaactggggagaatatttggaagagacaggagcaagtgctgctccccacacatcattcaaacacgttgaaatcagcattcagagcaacttccagccaggaatgaaattggaagtggctaataagaacaacccggacacgtactgggtggccacgatcattaccacgtgcgggcagctgctgcttctgcgctactgcggttacggggaggaccgcagggccgacttctggtgtgacgtagtcatcgcggatttgcaccccgtggggtggtgcacacagaacaacaaggtgttgatgccgccggacgcaatcaaagagaagtacacagactggacagaatttctcatacgtgacttgactggttcgaggacagcacccgccaacctcctggaaggtcctctgcgagggaaaggccctatagacctcattacagttggttccttaatagaacttcaggattcccagaacccttttcagtactggatagttagtgtgattgaaaatgttggaggaagattacgccttcgctatgtgggattggaggacactgaatcctatgaccagtggttgttttacttggattacagacttcgaccagttggttggtgtcaagagaataaatacagaatggacccaccttcagaaatctatcctttgaagatggcctctgaatggaaatgtactctggaaaaatcccttattgatgctgccaaatttcctcttccaatggaagtgtttaaggatcacgcagatttgcgaagccatttcttcacagttgggatgaagcttgagacagtgaatatgtgcgagcccttttacatctctcctgcgtcggtgactaaggtttttaacaatcacttttttcaagtgactattgacgacctaagacctgaaccaagtaaactgtcaatgctgtgccatgcagattctttggggattttgccagtacagtggtgccttaaaaatggagtcagcctcactcctcccaaaggttactctggccaggacttcgactgggcagattatcacaagcagcatggggcgcaggaagcccctcccttctgcttccgaaatacatcattcagtcgaggtttcacaaagaacatgaaacttgaagctgtgaaccccaggaatccaggagaactgtgtgtggcctccgttgtgagtgtgaaggggcggctaatgtggcttcacctggaagggctgcagactcctgttccagaggtcattgttgatgtggaatccatggacatcttcccagtgggctggtgtgaagccaattcttatcctttgactgcaccacacaaaacagtctcacaaaagaagagaaagattgcagtcgtgcaaccagagaaacaattgccgcccacagtgcctgttaagaaaatacctcatgacctttgtttattccctcacctggacaccacaggaaccgtcaacgggaaatactgctgtcctcagctcttcatcaaccacaggtgtttctcaggcccttacctgaacaaaggaaggattgcagagctacctcagtcggtgggaccgggcaaatgcgtgctggttcttaaagaggttcttagcatgataatcaacgcagcctacaagcctggaagggtattaagagaattacagctggtagaagatccccactggaatttccaggaagagacgctgaaggccaaatacagaggcaaaacatacagggctgtggtcaaaatcgtacggacatctgaccaagtcgcaaatttctgccgccgagtctgtgccaagctagagtgctgtccaaatttgtttagtcctgtgctgatatctgaaaactgcccagagaactgctccattcataccaaaaccaaatacacctattactatggaaagagaaagaagatctccaagccccccatcggggaaagcaaccccgacagcggacaccccaaacccgccaggcggaggaagcgacggaaatccattttcgtgcagaagaaacggaggtcttctgccgtggacttcaccgcgggctcgggggaggaaagtgaagaggaggacgctgacgccatggacgatgacaccgccagtgaggagaccggctccgagctccgggatgaccagacggacacctcgtcggcggaggtgccctcggcccggccccggagggccgtcaccctgcggagcggctcagagcccgtgcgccggccacccccagagaggacacgaaggggccgcggggcgccggctgcctcctcagcagaggaaggggagaagtgcccgccgaccaagcccgaggggacagaggacacgaaacaggaggaggaggagagactggttctggagagcaacccgttggagtggacggtcaccgacgtggtgaggttcattaagctgacagactgtgcccccttggccaagatatttcaggagcaggatattgacggccaagcactcctgcttctgacccttccgacggtgcaggagtgcatggagctgaagctggggcctgccatcaagttatgccaccagatcgagagagtcaaagtggctttctacgcccagtacgccaactgagtctgccctcgggaggtggcccattattgctgggatgcggtgttggtaaaggtttccaggactgaaactttgattttccgggatatgttaaatggtacagccactaagtatcaccagaaaaccagaagcccaggatcttctgcctccgccagcctgtgagctgtttccatgttttcaaagcacagcagcagtcgcttctggggagtgccagttaaagtcatgcatcagaccctgccagacgtgggcctgcttcttggctcacccacgttttgcctttctcctgccccaaatcaggcagctcccttggagcagggtttcctcagatgaggactgcattctttgaaaacaaagaatgtcgccaaggaagaaacctcacgccatgctgtagtgtttcctgtaatcacacgagcacatttatatatgcagtttcccatggataggcgtgtgaccctggttgagtggcacctgcggtttcatcttggtggcaactcctttgcaatgcagctggcagcgacatccttataaaaacatgtgctaaagctctgtcctctgttagaggtgccttttaggaatacggggagtgaaggaaggccggcaggcatctccatgcaactagatggtttgtttgtttgtttgtttgtttgttgttcattttgttgtgttttttgagacagggtcttgctctgtcgcccaggttgtaatgcagtggcgcaatctcagctcactgcaacctctctctcccgggttcaagtgattctcctgcctcagcctcccaagtagctgggattacaggcacccaccaccatgcctggctaatttttgtatttttggtagagacagggtttcaccatgttggtcaggctagtcttgaactcccaacctcaagtgatctgcccgcctcggcctcccaacgtgctgggattacaggtgtgagccactacgccccggcccaactggatggtttttgattgaagcctagaacatctgtagagacaaactctacccagtcttttctagaccctcaactatctccagtgttgttgtttaatcgtagccggatcagggagtgagtcttttaggcaaatgttggattatatatcaaaggaaaagcttagtttcagagaggaggaagggaaagagatgtgagggaagcatttcatcaaccagctacgttccccttagaaggatcactgcagcaggtcaccgagcaggagtccctctgagcgtcccttctgtctcgttctgccctagctggcagcatatgaaccaggcatgatgcagcaggagcagtgaatctggagtcagccacttggcaccctggtttcgctgagaacaaactctgagatcttgggtgacttctcatcactctggacctccattcctgtgaagtgacaggtgtggaccctgagggtgcggtggtgagcacactgtctcctgctggcattcaccccactcatgctggaaaggaagatccagatcgtacaaaaatgagaaaaagaaagaataagaagggtctggtcccagttctgactcggccattcttacagctctttctggctttgagtttgcttgtggaatttcctgggcagttgtgttaaatccgccaggtcacgtgcagacaaagctgtggctgcgagagttggctggcctcttggaccagaagccatctccatatcctcatgagcgattccatatctccactcagaccctgtggactacagtgttccgctgtggtggctgccaagatgccttcttaaacttatgcaaggaaaccaaaccctcccacagttcccaagcagacactggaagcagaggcttctcacccttcctgctttttcaccacaatcaccttgagctcgtcccttggactagagtctccacagttccagtaaaattctgcggtgggctgatgagctgcttgcatttctgtgacatttccagatatgattctcagtgggattttggaaactttgattgctcaagctcacccttcttaacattctgtaatggttacagatgagaatggaaaacacatattttatggatgaggcgttttggtctcccctgcagtcgatttctagaatcaagttttagagttcggctgatgcatctgcctggggacctcagatgggaggagtgtgtcagttgtaccccgacagaaatgtctctgggatctgtggctggcttgccccgggcatctctcctttaagctcaagttttgaactctctgcggttttccacccctgccttctcagccacatgcttttggccttaaacgctcagtcttgtggagttcaactctgtcaaacgattggaaagggcatccatttccagatctttggcattttccccgcgctgactctttgatgatccttcactgtggccttttcaagctcagctgttcctgttgtatttgagacgagggtgagggaatgtggtggccacaaaagaacagggacttgcagcacaaatgtcacttctgtctcccttttcagtggtagcatggaggaggaggtgctgcgttggagggaggggatcctccaggagctctctggagcccatctaggaagctagagtgtgtggcccgccaggagctcaggaaggatacagccactgtcgcaggggaaagtgtttgcttcccgtggagccaagcgcccaagactctccgtatccttcaccctgacagtttaacttcagcgtttctctgtgcagttgcggtcaccatgggtgagcactgtctgtgcacgtgccagggaggagatggctgggaccactgcacaggagggcgcagcctggcgtcaccatgaaagttgtctctgtgccatctctccggtccttgaggagagcccagaaagattttaggacccaggaggtgcttttcctccagctgttgccagtgtccttctgagcctggattctccggggatttccgtcgtggtggatggacttcacatcagcagcagttctggtacagaattgtaatgtgttttcatttctctgtaggattcacctctcaccagcgtctgtcttaaaggtagggccaatttcatggagcatttttctgtgtgtgtccttgttgcttttgccagaaaaagtggatttgacatgcgtgccccgatgccaccatagcccctaggccaacaatgtcatggtctaaacaccaaaaagagatgccccgcattccttccctggatggtaccgtttcttctccgtctctctttgatgattctttgggaccaaagtcctctccttagtgcgcctacttcctgtgggcatcatgccacttggaacttattggaactggcccgggagactctgcagtctgcgccgtttgaaaaccctgagaaagagatgccacctcaacttgaatcatgacagcccatcgctcagtctcaccctaaactcatggagcttgtttcagctcctcacttcttgactgtatttgtactatgttgaaaaaatatcctgtccacaaagacataagcctaacaacctagaaaaacaacagggtactactggcattacagaacttctttgcctttcaaaacaaaagcaaaacacagtgaacttcaccacggagctgcacagcgtggggaactcatccatcactttcaaaattagagtcatttgatccaagttggagtcagacacagtatttgagctgcacggcttctgggttctcccaccttatttgatcatattcaaaagattatttcctgtgtttgctttgatttgttcctcagtacattaaaatgatccacaccttgaacactgccctctctagaaggttgattttgatcagccttttgaagatgggtgtcgtttccctaacttatctcacagaattttgagtgttgtatttggcaagttctgagatttgccttctgtcttatgccaaacacccctttctaagagctgtccccgcttagttttagaagtactaggggttttcatacttattttatagaacacccatttatatttatttctgtatatagaactaaaaaaaacagtagtgttaaaaatctttgttgtggtttgagcatctttgctgcttttggattgagatggcgaatcaaggcttcacttcctctctcttctgtctttagaaagctgtgatcgtgcgtgcaattatttgaaaggcaacatagtcaattaagaaacctgtagttgttaaggaagaaattgttggcaagatatccatactgcccatatctcgttggtgcaataattaaatagcaaaggaaatctgtattggcaactattataattcaataattcttttgtttactgcccttttctgttcaagaattttctggaaattactccctttcacatggttgaactcttaagttgaccagttctcatagctctatcactagaatggtttgcagataccccaaacatactatgataaaatcaaattgtgctacttttgacccatgtaatttacctaaaagttgtaattgctgacagagtactgccttgaattttggtttaaaacctctctagtttcaatgacaagtaacaactcaaataattccatattgtttgaggaagaggccataatccttctgaattgttggcactaagtaatgggatttggcccagtaagtatgacggtcgtgtcgcctaaccaacgcagagcagtgctttttgtgtggctgaagcgatgtgctgacgaaaaaaggaaaattctaggacaatcgttggctaaaaatcaccttaggatgaaaaatttgaggcaaatttttttaaatgacagaaaaagataatcatctcacttgcttgaaacaggagccagcatgatctctggaagcatcaactatccctcgtcgtgattgttgaaagctctttcactgttttgcattctagtttgaatagtttgtattgaaattggattcctatcttgtgtatgtttttggtgcgtaaaagggaaaaattggtgtcattacttttgaaatttgcaggacgaagggcatgcttttggtttgctgtaagattgtattctgtatatatgttttcatgtaaataaatgaaaatctatatcagagttatattttaatttttattctaaatgaaaaaaaccctttttacttcaaaaaaattgtaagccacattgttaataaagtaaaaataaattct

SEQ ID NO: 16 - protein, 894 aa, Homo sapiens
MESTLSASNMQDPSSSPLEKCLGSANGNGDLDSEEGSSLEETGFNWGEYLEETGASAAPHTSFKHVEISIQSNFQPGMKLEVANKNNPDTYWVATIITTCGQLLLLRYCGYGEDRRADFWCDVVIADLHPVGWCTQNNKVLMPPDAIKEKYTDWTEFLIRDLTGSRTAPANLLEGPLRGKGPIDLITVGSLIELQDSQNPFQYWIVSVIENVGGRLRLRYVGLEDTESYDQWLFYLDYRLRPVGWCQENKYRMDPPSEIYPLKMASEWKCTLEKSLIDAAKFPLPMEVFKDHADLRSHFFTVGMKLETVNMCEPFYISPASVTKVFNNHFFQVTIDDLRPEPSKLSMLCHADSLGILPVQWCLKNGVSLTPPKGYSGQDFDWADYHKQHGAQEAPPFCFRNTSFSRGFTKNMKLEAVNPRNPGELCVASVVSVKGRLMWLHLEGLQTPVPEVIVDVESMDIFPVGWCEANSYPLTAPHKTVSQKKRKIAVVQPEKQLPPTVPVKKIPHDLCLFPHLDTTGTVNGKYCCPQLFINHRCFSGPYLNKGRIAELPQSVGPGKCVLVLKEVLSMIINAAYKPGRVLRELQLVEDPHWNFQEETLKAKYRGKTYRAVVKIVRTSDQVANFCRRVCAKLECCPNLFSPVLISENCPENCSIHTKTKYTYYYGKRKKISKPPIGESNPDSGHPKPARRRKRRKSIFVQKKRRSSAVDFTAGSGEESEEEDADAMDDDTASEETGSELRDDQTDTSSAEVPSARPRRAVTLRSGSEPVRRPPPERTRRGRGAPAASSAEEGEKCPPTKPEGTEDTKQEEEERLVLESNPLEWTVTDVVRFIKLTDCAPLAKIFQEQDIDGQALLLLTLPTVQECMELKLGPAIKLCHQIERVKVAFYAQYAN

SEQ ID NO: 17 - DNA, 4258 bp, Homo sapiens
  CDS (330)..(932)
agcgcaagtgtcctgttccctccgcgatgcggggcccaccgcgacgtccctccctcctgggcccctcctcaccgccgccccctgtagtttcagcctcagtgtcaggtgtgcgctgaatggaaaagggagattttgagacatcatgtcaacagaaatggagatgtgcactggggaaactgccggccgggccgctggcccgtggacgcctgggaggtggccaagggtcttgatctgttatccagactggagtgcagtggtgaatcagagcttattgcaaccttgaactcctgggctcaagcaatcctcctgcctcagcctcttgagccttcatgccccgaggactagcagacaaacaaggacctgaggaatgtgatgcagttgctcttttaagtctcatcaactcctgcgatcacttcgtggttgatcgaaagaaagtcacagaggtaattaaatgtcgtaatgagatcatgcactcttcagagatgaaagtatcttctacgtggcttcgagattttcagatgaagatccaaaattttctgaatgaattcaagaacatcccagagattgtggcagtatactccagaatagaacagctgttgacgtctgactgggctgttcacatccccgaggaagatcagcgagatgggtgtgaatgtgaaatgggaacttacctgagtgagagccaagtcaatgaaatagaaatgcagttactaaaggagaaacttcaagagatatatcttcaagcagaagaacaagaggtgttgcctgaagagctctcaaatcgactggaagtggtgaaggaatttctgagaaacaatgaggatcttagaaatggccttacagaagatatgcagaagctagacagcctctgtctacatcaaaaactggattcacaggaacctgggagacaaacacctgacaggaaggcctgaggttgcccgtcaacaaaaatcaggcatgttctgtgaaagtcagcatggcttccatctcagacatccttttctgtgcaaaaggaaaaagttaccagagtattgtacccaaacaaaaaggaatttttgttgttttgtcctggactttcctctaactctttggaactattttaatatttataaacttggggttgtataatctattgcaataaaaaatattagatactctatgccaaaacttgctaccaggccaggtgtgatggctcacacctgtaatccccagcactttgggaggccaaggcgggcggatcacctgaggtcaggagttcgagaccagcctggccaacatggcgaaaccccatctctacttaaaaaaaaatacaaatattagccgggcgtggtggcatgtgcctgtaatcccagctactcgggaggctgaggcaggagaatcgcttgaacctgggaggcagaggttgcagtgagctgagaccatgccactgtactccagcctgggcaatagagcgagattctgtctcccaaaaaaacaaaaaacaacaacaaaacttgctaccacccagggattttctgctatttaaaaggtgaatttcttttctggtactaaactgtagctgcttaacttagtaaaggctgtgtttggccaggcctgtgccagaggctcacctggagtgctccacccactggcaggcaagtcctattcctattcacccaggatccccaaggctgggctgggatataaatgttgggataggaaagaaatatttcctttttagaggaaagcaagaagaaacattgcctgaaaggtgattttctagtcatttccaattagtacagaaatgttactgcctctgggtgcagtggttcacgcctgtaatcccagcactgtgggcggatcacttgagcccaggagtttgagaccaacctgggcaagatggcgagaccccatctctacaaaaaaatttaaaaattacctgggcatggtggcacacacctttattctcagctactcaggtggctgaggtgggaggatcccttgagcccaggtggtcaaggctgtggtgagctatgatcatgccactgcactccagcctgggtagcagaacaagaccctgtctcaaggctgtggtgagctatgatcatgccactgcactccagcctgggtagcagaacaagaccctgtctcaaaaaaaaaaaaaaccccaaaaactgttactactaggttggagtagcctaaggcagtagggcaaggaggtgggcccaggctggtctgtggggagctggagaatggtgactcgagtgaccagtggacaggcaggacccaggccagcattacaagcggggatgtcaggcaggagcaggggagcttctcgcttggcagctggtttatggtacacttttgaaaagtaagctcccagggcctggccctcacatgctcagtgaatatttgactgaagggtcccctcatagcttgggagtattcacaggcctaaatgttagttatactattagctaagcctggtcctttgttacgaaatttaaaaatgaaattgcaacattcttgtggaaattcaaagacatcattttctttcaacaaattcacacattcgttcatgcttcttctataccttttacccctaaagtcatccactcctgagttcctcctgctggtttcccaaactgcgcaatgactgccccatcataggcagtggtccgtggagtcggcctcactttcgcctttccccgcatctacttgtttccaaggccagtggtacttaactgggtcaagttgcctgtggaccttcaggaacagaattgccatgttcctcactgcctgcaggaaaggctccattccaagcccagtgaagatgtgtgcctatccagccgcccaccaaggatgtcatctgtagaatgggtggagggcaggggtttatttggtgtatatttttacattaaaatgcacttaatatcactttgtaaagcccagatgagtgcaaatgtgcctgtaacttcctcctttaatctgtccaggtagtatttagtctttagtcttacattttctttctccctttatttcatgaaattccttgagaaaacttcaacagtaaagaaagaaatttcgttcatctcacaactcttccaaacgaggaaacttagtgaaatatttcagagcttctagatgtgaggtacaaaacttgggatcaaatggaatcttgattcactaaccaatttaagatctgacttctaattttaggaactttgggttatgaacgcttccattttatacctgtgtctagttagtttctgcctatctatccaagaagcttttatcaagggtccaccatgtgccagccactgaagtagatataaatataaggatgtgtaaggtatggatgatggtatacgaactgtcatcttactggatttgtccgctctgttaaagatacggttccgaaaactttttaaagccctagagagggctttaaggcaatgtagcatcatatatagaggcatcaacctgttcatatctttctatttaacagaactgtgcacctgggcacaagggtgtgcacaacaggatgtgtacagcagcactgttaaagtgtagcacatccatactacaggatcttatgcaactgttggaaagaatgaagcgatgctgcactgtggtcatgcagtgatctctaagacatattaactagaaagcaaaaggtttaacaatgtatagcagctgggcgcagtgactcgcgcctgtaatcccagcactttgggaggctgagtagggcggatcacctgaggtcaggagtttgagaccaacctggccaatgtggcgaaacgctgtctctactaaaactacaaaaattagctgggcgtggtggcgcgtgcctgtaatcccagctactcggcaggctgaggcaggagaatcgcttgaactggggaggtggaggttgcagtgagccgagatcacaccactgcattccagcctgggtgacagagggagactccgtctctaaaaaacaaccccccccccccaaaaaaaaaaatgcatagcaagctgtaatgctctttgtgttttagaatagtagaggtctggaaagttgtttgcttttccccagtttttttttgctgtgttacctctgaagggaattgaggtagaggggagagttagaaggaatattcggcttttctattttatatcctcctaggtgaaatttttacaacaaacatgtactggtgtattttgaaatgtttttaaatttttgtatttcaaaataataaaatataaattccaactgaaaaaaaaaaaaaaaaaa

SEQ ID NO: 18 - protein, 200 aa, Homo sapiens
MPRGLADKQGPEECDAVALLSLINSCDHFVVDRKKVTEVIKCRNEIMHSSEMKVSSTWLRDFQMKIQNFLNEFKNIPEIVAVYSRIEQLLTSDWAVHIPEEDQRDGCECEMGTYLSESQVNEIEMQLLKEKLQEIYLQAEEQEVLPEELSNRLEVVKEFLRNNEDLRNGLTEDMQKLDSLCLHQKLDSQEPGRQTPDRKA

SEQ ID NO: 19 - DNA, 5211 bp, Homo sapiens
  CDS (156)..(950)
ccacgaccaatgatatgaaatacaacgatggtttttcatatcattggtcgtggctgtagtccgtgcgagaataatgatgtatgctttgtttctgttgagtgtgggcgcttgccccgggggtctgtcgcgtcgcaggcgccgccggagccctggccatgaggaccctgtggatggcgctgtgcgcgctgtcgcggctgtggcccggggcccaggccggctgcgccgaggccgggcgctgctgtcccggccgggaccccgcctgcttcgcccgcggctggaggctggacagggtctacgggacgtgtttctgcgaccaagcctgtcgcttcaccggggactgctgcttcgactacgacagggcgtgcccagctcgcccgtgcttcgtgggggaatggagcccctggagtggttgtgcagaccagtgcaagcctacaacccgtgtgcggaggcgctcggtgcagcaggagcctcagaacggcggggcgccctgcccacccctggaagagagagctggctgcctggagtactccaccctgcagggccaggactgcgggcacacctatgttcctgcctttataactacctctgcattcaacaaggagagaacacgacaagctacgtctccacactggtctacacacacagaggatgctggatactgtatggagtttaagacagagtccttgactcctcactgtgctctggaaaactggcccttgactagatggatgcagtatctccgagagggatacacggtgtgtgtggattgtcagcctccagctatgaactctgtgagccttcgttgttctggagatggcctggactccgatggaaatcagactctccattggcaagcaattggtaatcctcggtgtcaaggaacttggaaaaaagttcggcgagtagaccagtgttcttgtccagctgttcacagttttatttttatatagatggtgatataaatatttccaaatgcatttgtaaacattctaaatattctcaagtcatgttcaatgtttcctaaaccttcaattttggccaaagtccccaaacacatcattgccacactctgaagtagagaaagaaaatttaggggccagttctcaaggaacacaggtcctttatttttattttaactaagttgaagacccactcaaaaagctcttgtggttttatgttcttgacctttcaactggagtcctctcattcagcaggtggcccgtgagacacagaatacatgtctgtttgctaaagtaaaattactgtaactcagtccgattattggtgacggaagtgtcatttaaggggatctatgttttgaaccttgcagtctattttataatcttttaaaagtttctcactttaaaacatgtatacaggtatgcacactctacacatatatttccatatatttaatattccataaattctgaaataatttcaagcaaattatcacaaataatttttccacagggcaaattatttaaaattttagtaagcattctgtaatgaaaaaccaagttatactaaaaacattttttgaagaagaaattttttttttgcttaagaagtgaaaggatcaaatgctcattgtaggtcaatgggagttcttttaaatgttataattttcatgaaaagaaatgttgacaccagtgaatgaaacaattgctttcattctgaaaattctaccaccatttgcatctaagattatttccaaggcttaaagcctgaagctgaataaaataatctttcagagtccagcttcaagtttagttgatgtaagctcactatttttttcctaccgcatgcattttctaatgtttggggtggatggtgtgtcggttatggaaggcatagacgtcattacaggtgctacgatctcacacacacacaaggaaatgttagtctccttattttatgattggaaaatcaatgacctagaggcaaaatggcatgtttaaggacctgggatgacaagtcattctgcagtcagccacagagccaaatttggactcctcaaccagaactccatgaaaagcctgactttgccaaacactgtgctggaaaagctaagcccctttcatttgtgaagtaaattttaaattcaagatatttagtttagagaattgagtcttgagatgtaaactacatgagatttctttggtttcaattgaataatattcactaacaaatgatttactaaaatacgtatttcttggtccttatcatgtaatgacagattcacaacagcaataaggatggagatttccccaataattaataacaccgagagtagcaatattttttactgtattttcatttaattgtcaaagttgtttttgtggaaaattaaattcttcagaaattaataataaaatatgtgagtctcatcaaggctagcaactttaagtcagtattaacattttcatactgtttctaacatttattaagctagatttttgaagctatgtctgggtaatttccaatgttacttaaaagtttttttttctttgagacagagtctcactctgttgcccaggctggagtgcagtggccccatctcggctcactgcaagctccgcctcccgggttcacgccattcttctgcctcagcctcccaagtagctgggactacaggcgccgtcatcacgcctggctaattttttgttttttttttttagtagagacagggtttcactgtgttagccagggtggtctcgatctcctgacctcgtgatccacccacctcggcctcccaaggtgctgggattatgggcgtgagccactgcgcctggcctgttacttaaaatttttatactagtaaataaactttttgttcacatttataaaagctctatatttgtttgactagaaaataacccaaattttctggaccagtcagagaatgataaaatttcaccaaaaataaaacaaaaaaatccttcagaactgataggtataggtacaacaactactaatagcaataaagcaatggtgcttttatagttccgatatgtgttataacatttaacataattgaggtgtataaaataaatttttcaccttatgcatttaatttttggtggatgaccagtaattgctttcttattgaagttaaatttttgttatttctattcttgagaaaattttatgggctagaggagttaactgttaaataacagttttgaaaagcttgttattaattttagcaaagcccaagggaaatagttcagaactgaaacatgtagtccaagtgatgaatgaaaaaccaaaagtttttattttcaaatttaaaaatcaaagtatataagacctcttggcactcttttttaataagtaccttagtcttagggccctatccccaggcactctaaaaagatttacgaactctaaaatcccttaatagactgattttcaaaagtgctcatggaaaataattcgaacaaaaataaatacagccaccaggaaagaagggaaaagaagcaagggataatggccatttttgaagagaaaagtgaaaaacaagtgaaggaaaaaaagaatagaaaaaggaataaggtaactataatcacagtattctttttggagttttgctttggtttttattcgagaatgttacaattaagcataaaccgcaacagcaataatttaaagtgtttggttatcatctggcataaaaatttcagttagctatcttttctagatgtgcacaaaattgatgttaataaaactggttcctgccctctaagaatattctagtgaaggttcatacaaacaaccacaagttggcttagaaaacaaacacctgggccttctgaggtccaagcaatcagacaaagagcaccgtgagggaggtcacactctaggtcagggtgaaagacagacagacacacgtttaccctctctccttcctcatatcccactgaagagacacgattcacagaagagaataagtccacagcagatgagaagtgaggtgggcagaatcagcctacagttcgaataattttttttacgattgaaagcaaatgataacctatgatagatgaaccagaataaaggggccatatcctttaatacggggtacagaagaagctggaatcagagatgagagccattcttggtggggcagtcccaccaagcaccaaaggatgaataaaaagactctcagccctagagacaaatttgtgaaattgaaaatgaaggataaagaaaagaccatataaccttccagagagagaacaacagattacctgtaaaggaacaggagtccccatgaagacagcactcttctcatcagcatcagtgcatgcttaacatctaacagaagaaagcctccaagctctgagggaaaatgaatgtgaaactaacattcgatagcagccatcactaataaagtgtgaaagtaacatacagacttttaaaagcacaaggatcggaagctttattaaaaggtcctaagcaggacattcttttttctggttaaacatggcaggttggacacatatatttatgtcccaattcccaataaaattacagttaagggattttcaaagccatccccccaccaaaacaacaacaaagcccatatataatgaggagttcaagagagaaaacgcagaagagatggccacatttcaggaccagaaaagatggctactaaaatcggaggagaaagccaaacacctagatctacgggtgaatttgagtaagcaaggtaatttctagcactctggaaaggctctggaactgaaggttccaggtatttccttcaatggaatgaatggctgatggttggtctaagaagtcagagctttagattccctcccccatgctgcgggtcaacagcctgacagaaggttcagtctatggaggagtttggattggaaacaccagtcacaaggacagaatgaagcctgaatgaaagtctctattctgactggtgacccctggttcccttttccatgtgctctgagaatattgcaagcagggatagatatggcactccttcactccctcactgccactcctctcatccctacccaccagtttgttagattcctctctaaggaagtggattatccaagagaaatgacaataggtgttgacagttgaggatcccctaatgaaagagccatgtctgtgtctaatcactacaggcatatcgctacctggatgtctttgttcagcctgctatatgaaaaaaaaaaa

SEQ ID NO: 20 - protein, 264 aa, Homo sapiens
MRTLWMALCALSRLWPGAQAGCAEAGRCCPGRDPACFARGWRLDRVYGTCFCDQACRFTGDCCFDYDRACPARPCFVGEWSPWSGCADQCKPTTRVRRRSVQQEPQNGGAPCPPLEERAGCLEYSTLQGQDCGHTYVPAFITTSAFNKERTRQATSPHWSTHTEDAGYCMEFKTESLTPHCALENWPLTRWMQYLREGYTVCVDCQPPAMNSVSLRCSGDGLDSDGNQTLHWQAIGNPRCQGTWKKVRRVDQCSCPAVHSFIFI

SEQ ID NO: 21 - DNA, 1345 bp, Homo sapiens
  CDS (28)..(759)
cggaagtgggaagagagaaaggttgtgatggcggctatagctgcatccgaggtgctggtggacagcgcggaggaggggtccctcgctgcggcggcggagctggccgctcagaagcgcgaacagagactgcgcaaattccgggagctgcacctgatgcggaatgaagctcgtaaattaaatcaccaggaagttgtggaagaagataaaagactaaaattacctgcaaattgggaagccaaaaaagctcgtttggagtgggaactaaaggaagaggaaaagaaaaaggaatgtgcggcaagaggagaagactatgagaaagtgaagttgctggagatcagtgcagaagatgcagaaagatgggagaggaaaaagaagaggaaaaaccctgatctgggattttcagattatgctgctgcccagttacgccagtatcatcggttgaccaagcagatcaaacctgacatggaaacatatgagagactgagagaaaaacatggagaagagtttttcccaacatccaatagtcttcttcatggaacacatgtgccttccacagaggaaattgacaggatggtcatagatctggaaaaacagattgaaaaacgagacaaatatagccggagacgtccttataatgatgatgcagatatcgactacattaatgaaaggaatgccaaattcaacaagaaagctgaaagattctatgggaaatacacagctgaaattaaacagaatttggaaagaggaacagctgtctaatcccttcaagaactgtttatagaagcttgagaatggggtaaaaatttctgctagcaaaatcaagttctttttgaaattttatcagtaatccagaatttagtagtccatgccttctcactcagcatttagaaataaaaatgtggtttcttaaacgtatatcctttcatgtatatttccacatttttgtgcttggatataagatgtatttcttgtagtgaagttgttttgtaatctactttgtatacattctaattatattatttttctatgtattttaaatgtatatggctgtttaatctttgaagcattttgggcttaagattgccagcagcacacatcagatgcagtcattgttgctatcagtgtggaatttgatagagtctagactcgggccacttggagttgtgtactccaaagctaaggacagtgatgaggaagatggcagtggccaccggaggactggagcagtccctcctcatggcggcctgtgaccaaggtcggggaggagtggagctatccttccatgatctgatcatgtacagttccctttttaaaaagcaataaatgcttgggattagaatttcta

SEQ ID NO: 22 - protein, 243 aa, Homo sapiens
MAAIAASEVLVDSAEEGSLAAAAELAAQKREQRLRKFRELHLMRNEARKLNHQEVVEEDKRLKLPANWEAKKARLEWELKEEEKKKECAARGEDYEKVKLLEISAEDAERWERKKKRKNPDLGFSDYAAAQLRQYHRLTKQIKPDMETYERLREKHGEEFFPTSNSLLHGTHVPSTEEIDRMVIDLEKQIEKRDKYSRRRPYNDDADIDYINERNAKFNKKAERFYGKYTAEIKQNLERGTAV

SEQ ID NO: 23 - DNA, 2434 bp, Homo sapiens
  CDS (251)..(889)
ggcggggcggaggcgccgcggcggctgttattgttcggctgggctcggtcgggcgctgtctccctcggctctgcgggtgtcagttcgtccggcttcctcacagcccctcactcccggcggctgacagcagcagcggcggcggcgggcggcgcctggcgtttcgaggctgagcggcaccggggttggggcgcggaggaggagcagcagcgggaggaggagccgtgtgccctggcactgagcggccgcggccatggcgtacgcctatctcttcaagtacatcataatcggcgacacaggtgttggtaaatcatgcttattgctacagtttacagacaagaggtttcagccagtgcatgaccttactattggtgtagagttcggtgctcgaatgataactattgatgggaaacagataaaacttcagatatgggatacggcagggcaagaatcctttcgttccatcacaaggtcgtattacagaggtgcagcaggagctttactagtttacgatattacacggagagatacattcaaccacttgacaacctggttagaagatgcccgccagcattccaattccaacatggtcattatgcttattggaaataaaagtgatttagaatctagaagagaagtaaaaaaagaagaaggtgaagcttttgcacgagaacatggactcatcttcatggaaacgtctgctaagactgcttccaatgtagaagaggcatttattaatacagcaaaagaaatttatgaaaaaattcaagaaggagtctttgacattaataatgaggcaaatggcattaaaattggccctcagcatgctgctaccaatgcaacacatgcaggcaatcagggaggacagcaggctgggggcggctgctgttgagtctgtttttactgtctagctgcccaacggggcctactcacttattctttcaccccctctcctcctgctcagctgagacatgaaactatttgaaatggctttatgtcacagaagactttaatccgtcaaattcttgtataactttgaataaatggttaatgttcacttaaaagacagattttggagattgtattcatatctatttgcatttgatttctaggtcaattgatgtgattatttttgttaaatgttgtcttgtgcccttaactacgaactgaattgtattaaacactacaaagtcatcttgagtattttaaatcggtttgtgtagttaggtttncccaacatctgtggttacctaatgtttaatattatagaactgtcctcagaaactttgtcaattttcacggctataaggaaacagaaggactcttttaattctgtatttatcatttactttctgtatatatagtttaataacctgcttgggtgtaatttgccaagcttgaattctttaatgcatttgcataaattctatactgtttagagcttaaagctacagaagcattgttaggaattgcttggacactgaattttaaactttttgacattgttaacaagcatgttcatcttttcttgtcactagtccaagaaaaatatgcttaatgtatattacaaaggctttgtatatgttaacctgttttaatgccaaaagtttgctttgtccacaatttccttaagacctcttcagaaagggatttgtttgccttaatgaatactgttgggaaaaaacacagtataatgagtgaaaagggcagaagcaagaaatttctacatcttagcgactccaagaagaatgagtatccacatttagatggcacattatgaggactttaatctttccttaaacacaataatgttttcttttttcttttattcacatgatttctaagtatatttttcatgcaggacagtttttcaaccttgatgtacagtgactgtgtaaaatttttctttcagtggcaacctctataatctttaaaatatggtgagcatcttgtctgttttgaaggggatatgacaataaatctatcagatgggaaatcctgttacaaagtagaaaagctttagtaatttactcagtgtggtggttttatcctttttttctttttctcccttggtctataatgaaattgttacagcagtgcaaaataaaatcctatgtataaaagtgttctttttttttattatgaccacctcttttttaatgtatttttagtccacttacagcttttacatgggtttaagcatgttttttaaaagggtcagaatggttaacactcaaccctttttaaaaattttgctaaaatgcgacaaatctcaccatactgaaattatttttgttgatggtgtaagcagtgtaagcaagtggttttctcctgaactagcacaaaagcacttatgcctgaaagaaagcataaagaagttctaactctgaaactaactactttcatttcgctcatggccttcaactttctacaggtc

SEQ ID NO: 24 - protein, 212 aa, Homo sapiens
  misc_feature (1229)..(1229): n=A,T,C, or G
MAYAYLFKYIIIGDTGVGKSCLLLQFTDKRFQPVHDLTIGVEFGARMITIDGKQIKLQIWDTAGQESFRSITRSYYRGAAGALLVYDITRRDTFNHLTTWLEDARQHSNSNMVIMLIGNKSDLESRREVKKEEGEAFAREHGLIFMETSAKTASNVEEAFINTAKEIYEKIQEGVFDINNEANGIKIGPQHAATNATHAGNQGGQQAGGGCC

SEQ ID NO: 25 - DNA, 3174 bp, Homo sapiens
  CDS (91)..(1323)
cactttggaatcccatcttgagactcgctaagcgtcccagccgcatccctcccgcagcgacggcggcccgggacccgcgggctgtgaaccatgaacacccgcaatagagtggtgaactccgggctcggcgcctcccctgcctcccgcccgacccgggatccccaggacccttctgggcggcaaggggagctgagccccgtggaagaccagagagagggtttggaggcagcccctaagggcccttcgcgggagagcgtcgtgcacgcgggccagaggcgcacaagtgcatacaccttgatagcaccaaatataaaccggagaaatgagatacaaagaattgcggagcaggagctggccaacctggagaagtggaaggagcagaacagagctaaaccggttcacctggtgcccagacggctaggtggaagccagtcagaaactgaagtcagacagaaacaacaactccagctgatgcaatctaaatacaagcaaaagctaaaaagagaagaatctgtaagaatcaagaaggaagctgaagaagctgaactccaaaaaatgaaggcaattcagagagagaagagcaataaactggaggagaaaaaaagacttcaagaaaaccttagaagagaagcatttagagagcatcagcaatacaaaaccgctgagttcttgagcaaactgaacacagaatcgccagacagaagtgcctgtcaaagtgctgtttgtggcccacaatcctcaacatggaaacttcctatcctgcctagggatcacagctgggccagaagctgggcttacagagattctctaaaggcagaagaaaacagaaaattgcaaaagatgaaggatgaacaacatcaaaagagtgaattactggaactgaaacggcagcagcaagagcaagaaagagccaaaatccaccagactgaacacaggagggtaaataatgcttttctggaccgactccaaggtaaaagtcaaccaggtggcctcgagcaatctggaggctgttggaatatgaatagtggtaacagctggggttctctattagttttttcgaggcacctaagggtatatgagaaaatattgactcctatctggccttcatcaactgacctcgaaaagcctcatgagatgctttttcttaatgtgattttgttcagcctcactgtttttaccttaatttcaactgcccacacacttgaccgtgcagtcaggagtgactggcttctccttgtcctcatttatgcatgtttggaggagctgattcctgaactcatatttaatctctactgccagggaaatgctacattatttttctaattggaagtataattagagtgatgttggtagggtagaaaaagagggagtcacttgatgctttcaggttaatcagagctatgggtgctacaggcttgtctttctaagtgacatattcttatctaattctcagatcaggttttgaaaagctttgggggtctttttagattttaatccctactttctttatggtacaaatatgtacaaaagaaaaaggtcttatattcttttacacaaatttataaataaattttgaactccttctgtataaatgggtcatttttatttttaatgaaaagttattggggttttctctcttgaagggtctcattttaattcccttttccaggccgtatagatcaaatatagtactgtcattactgttggctcttgttttggtcttgacttactaatagtgttaccctgattttcagagggggacagtttatctccagaaaggccaatgtttgtatacacatcagctagacacaaatatagacatcatatgtagtttgtacatgtttcagaaacttgttttttctttgctctgtgtaacctatttcctattgctagttcagttggctttcttattcacttctgtgaccctgaaccagttctcagaccctagagtgtaagagcattgattttctacgctgtgtaatctagctcaatccctctgtcccctccgcctcaccgtcccccagccaccacattgtatagcaaaagcattacattcaatcctagaacaaaggtaaatacaacaaatcatctttgcagctggacaactaataatactttgcagcattaagagatcttctgtgttaccagtcactctgttgaaatgaactttccgaatctctttattcaggaaaacatgggggttttgaaatttcttgggccaagagacatatctgaggggttcgcagagctaggcaagggtgcactaggaaagggccacattggtgggtgggggagtaacagagaacagatggtgtcaaggaagtttctctggagtaaataatgtggatattcnttggttccctctcctccgccagctgaagctggttagtgctgttgacactaatataaaatggtttggtccatttgaaatccttgtcattgccttatatgggggaaactcaatcccccagcctgtgttggaaacatcaccaaactgattgtaaatgtgcggctgtagcagacattttagtgtggtggtgtgcagccatttcggccctacacctgccagcctggctaccttacagttgtgttccgatttttgcgtctatgcttggtgtgcctcacttgctgcattttccagcatgcaaccaggagttgacgtaggaaaaagggatgctttcttactttggaagctctcagggaagttggtgtcaatttctcctccactgcctggcctaccctgcactcccaaagattttgtgcagatgggtagttccatttttttaaaaattgtgcagatatggaaaattgtgacttacttcatgaccagaactatctagaatatgtgtgggggtataaacatcttgcttaaccaaatatctatgtaggcagaggtaaccaggagagaagcaagacttgctgcctaaaggagcccaccattttacttttcacatttaatctgccacgttgaatcaattggaataaaacctgactcgcaggtgactggacaggaaatcccaaaaccatttctatgcttaattttaacgtccccccgcttttttttttgtagaaaataaaaacaagaaaatcgttccaatgtaagatgtttgttatagaaactttaggcaatacaggtgtgtaataaaaatgtttaataaacttctaaacactttt

SEQ ID NO: 26 - protein, 410 aa, Homo sapiens
  misc_feature (2353)..(2353): n=A,T,C, or G
MNTRNRVVNSGLGASPASRPTRDPQDPSGRQGELSPVEDQREGLEAAPKGPSRESVVHAGQRRTSAYTLIAPNINRRNEIQRIAEQELANLEKWKEQNRAKPVHLVPRRLGGSQSETEVRQKQQLQLMQSKYKQKLKREESVRIKKEAEEAELQKMKAIQREKSNKLEEKKRLQENLRREAFREHQQYKTAEFLSKLNTESPDRSACQSAVCGPQSSTWKLPILPRDHSWARSWAYRDSLKAEENRKLQKMKDEQHQKSELLELKRQQQEQERAKIHQTEHRRVNNAFLDRLQGKSQPGGLEQSGGCWNMNSGNSWGSLLVFSRHLRVYEKILTPIWPSSTDLEKPHEMLFLNVILFSLTVFTLISTAHTLDRAVRSDWLLLVLIYACLEELIPELIFNLYCQGNATLFF

SEQ ID NO: 27 - DNA, 2526 bp, Homo sapiens
  CDS (386)..(784)
cccagacgccaaggttgcgggtcatggagtcccgaaccctcctcctgctgttctcgggagccgtggccctgatccagacctgggcagattacaattacaatcaaggcagaaatgatctcatttttacattacaactctggaaaaggcaatagactgagatgcaagtgtgcccccaagtgatgggcagaaggagagaaggtgttttggatgcattctagaacacaggtaatctaaggagagttgatcaaggccgtggaagagtcctccagccactattggccatcaaaggagtcctctgtgtcccaggaatggtcctgctttggtgtccctggtgtgacccatcacccgctgggaacagcctgagagaagtagggcctctgcaccaatgctgctgaggatgtcagagcacaggaacgaggccttgggaaattacctggaaatgcgactgaaatcttccttcctgaggggtctgggctcttggaaatcaaaccctctcaggttgggtggctggacgattctcctcacacttacaatgggacaaggggaaccaggaggcccccaaggggatccctgggttccacacgaactcctcctaccctcattgtgtgacagcagccatgcctcctcctgtggatcaggatctattacctgtgcctggagaggaggggactcctcttctcacccgctggtctctggacacatactgtccaattcccctgtggcagctgtaatgtgtagttcaatgggcactcatttgtccccttttaagggtaccctcctttagaatccaggaccttctaccctgcagagtgtggttttgggagagaagtgcaaaatcccacgacaggtgagttgaaggaatgggatatggagccacatccacttccaccccttggtatctggacccacgtgttcttcctactgagattacagaactgtagagatatctttgatttttaaaatgcaccatgtcctgaaagatggcaccctcccacccgcagagtgcttcctgcaagctggcgttgagctgtgcctatagaagctcttttcaacattctttatggtcagcagcccttggttggtgcagatggtgataggaccagtgggtcccacagcatggccacactgcacctccttcgctgtcaagtgggtcccccacgaagatactgcacggagagcagtgccaagcctgtggatcaggaatatcaacagcccccagagagtggtgctggctgagggtctgagagcaggacaggaaaacccacctatggaataggtgcctatccctgtgaagatgaacctctggcccttccaggatggaaggagtgcaatgtagtcaactcctcacttagggtctggttggtcacctaaagaaatagagccctaccagggaagatcattgggttcaaatgctgatgagtaggacatttagaggtggcagtgtctggatctaccttggtaggagggagtcagtactgttggacccataggtagcctcatccctgccactgtgtttgctccatttatgtacccatcctacccggcctgggctgacccatggggaaggctggctaatttcagtgcttctgcttggttgttcagggccatttcaggtttgggtgttttctggggatgttaacatgggattcaggctcaactcacaagaaacttttccatctcatgatggatgctgttgggcatgtccaatgtatgacttcatgagttacacagatgctaattcgtaggggcacttggaatcacatggttgttttgtgtcccatggtcaagcattctatcttatcagggcctacagtaacatgccaaaagttgcttccaacatatttctctgctttggatggggcatatttctgtgctgtggatgacatggccttactccagaatcccaggccctccactgtgactctcctactggtgcttggttcagctccaccccaaatcttaccccaccactggcactttcagcaccagggggtctgaaggatggtgactgcgccatggcctggatctgctgcagtgtcctttcctgtggaggctccactcaaagctggcatcctcctatgtcacctagagtgtgggtcaaagcaatacacctacatgtagaatgtgatgtcagaactcaaacaggctcaccaggcagtgtgcttccttccttgcatgaggatgcaagatgcaacagtttgtctttcacattggaagggacacccctggatgcccctaaccactagacctgtaaaacttcactgcagtggccacttctgaatctctgtaaggtttatttatcttcaccctctggagagaagatgttttaccaaagcctctagtgtaccttcctcctcttactcatccatcccagtcaacatgatgttgtcaatgaaataaaggaatttaatattctatagtatatccaggttctccagatctcttaagactgtactatagaggcctggggaattataatagccctgaggcaaactatgaattaaagtgttgtggatcccacatgaa

SEQ ID NO: 28 - protein, 132 aa, Homo sapiens [sequence omitted]

SEQ ID NO: 29 - DNA, 1384 bp, Homo sapiens
  CDS (200)..(1333)
ctttctccctcctcagttggccgagtcgtcccgcgcgcaccgcctccgcgcgcctatgagaatgaggtggtaacgggcccccggatgaccccgcgtcaccactgtgaggcctacagctctgccggggaggaggaggaggaggaagaggaggagaaggtagctacagcaagctgggtagcaggcagatccaaaggatatcatgaagtttccagggcctttggaaaaccagagattgtctttcctgttggaaaaggcaatcactagggaagcacagatgtggaaagtgaatgtgcggaaaatgccttcaaatcagaatgtttctccatcccagagagatgaagtaattcaatggctggccaaactcaagtaccaattcaacctttacccagaaacatttgctctggctagcagtcttttggataggtttttagctaccgtaaaggctcatccaaaatacttgagttgtattgcaatcagctgttttttcctagctgccaagactgttgaggaagatgagagaattccagtactaaaggtattggcaagagacagtttctgtggatgttcctcatctgaaattttgagaatggagagaattattctggataagttgaattgggatcttcacacagccacaccattggattttcttcatattttccatgccattgcagtgtcaactaggcctcagttacttttcagtttgcccaaattgagcccatctcaacatttggcagtccttaccaagcaactacttcactgtatggcctgcaaccaacttctgcaattcagaggatccatgcttgctctggccatggttagtctggaaatggagaaactcattcctgattggctttctcttacaattgaactgcttcagaaagcacagatggatagctcccagttgatccattgtcgggagcttgtggcacatcacctttctactctgcagtcttccctgcctctgaattccgtttatgtctaccgtcccctcaagcacaccctggtgacctgtgacaaaggagtgttcagattacatccctcctctgtcccaggcccagacttctccaaggacaacagcaagccagaagtgccagtcagaggtacagcagccttttaccatcatctcccagctgccagtgggtgcaagcagacctctactaaacgcaaagtagaggaaatggaagtggatgacttctatgatggaatcaaacggctctataatgaagataatgtctcagaaaatgtgggttctgtgtgtggcactgatttatcaagacaagagggacatgcttccccttgtccacctttgcagcctgtttctgtcatgtagtttcaacaagtgctacctttgagtgtaaactaaggtagactactttgggaa

SEQ ID NO: 30 - protein, 377 aa, Homo sapiens
MKFPGPLENQRLSFLLEKAITREAQMWKVNVRKMPSNQNVSPSQRDEVIQWLAKLKYQFNLYPETFALASSLLDRFLATVKAHPKYLSCIAISCFFLAAKTVEEDERIPVLKVLARDSFCGCSSSEILRMERIILDKLNWDLHTATPLDFLHIFHAIAVSTRPQLLFSLPKLSPSQHLAVLTKQLLHCMACNQLLQFRGSMLALAMVSLEMEKLIPDWLSLTIELLQKAQMDSSQLIHCRELVAHHLSTLQSSLPLNSVYVYRPLKHTLVTCDKGVFRLHPSSVPGPDFSKDNSKPEVPVRGTAAFYHHLPAASGCKQTSTKRKVEEMEVDDFYDGIKRLYNEDNVSENVGSVCGTDLSRQEGHASPCPPLQPVSVM

SEQ ID NO: 31 - DNA, 1510 bp, Homo sapiens
  CDS (23)..(1360)
ggactcccttttctttggcaagatggcggagtacgacttgactactcgcatcgcgcactttttggatcggcatctagtctttccgcttcttgaatttctctctgtaaaggagatatataatgaaaaggaattattacaaggtaaattggaccttcttagtgataccaacatggtagactttgctatggatgtatacaaaaacctttattctgatgatattcctcatgctttgagagagaaaagaaccacagtggttgcacaactgaaacagcttcaggcagaaacagaaccaattgtgaagatgtttgaagatccagaaactacaaggcaaatgcagtcaaccagggatggtaggatgctctttgactacctggcggacaagcatggttttaggcaggaatatttagatacactctacagatatgcaaaattccagtacgaatgtgggaattactcaggagcagcagaatatctttatttttttagagtgctggttccagcaacagatagaaatgctttaagttcactctggggaaagctggcctctgaaatcttaatgcagaattgggatgcagccatggaagaccttacacggttaaaagagaccatagataataattctgtgagttctccacttcagtctcttcagcagagaacatggctcattcactggtctctgtttgttttcttcaatcaccccaaaggtcgcgataatattattgacctcttcctttatcagccacaatatcttaatgcaattcagacaatgtgtccacacattcttcgctatttgactacagcagtcataacaaacaaggatgttcgaaaacgtcggcaggttctaaaagatctagttaaagttattcaacaggagtcttacacatataaagacccaattacagaatttgttgaatgtttatatgttaactttgactttgatggggctcagaaaaagctgagggaatgtgaatcagtgcttgtgaatgacttcttcttggtggcttgtcttgaggatttcattgaaaatgcccgtctcttcatatttgagactttctgtcgcatccaccagtgtatcagcattaacatgttggcagataaattgaacatgactccagaagaagctgaaaggtggattgtaaatttgattagaaatgcaagactggatgccaagattgattctaaattaggtcatgtggttatgggtaacaatgcagtctcaccctatcagcaagtgattgaaaagaccaaaagcctttcctttagaagccagatgttggccatgaatattgagaagaaacttaatcagaatagcaggtcagaggctcctaactgggcaactcaagattctggcttctactgaagaaccataaagaaaagatgaaaaaaaaaactatcaaagaaagatgaaataataaaactattatataaagggtgacttacattttggaaacaacatattacgtataaattttgaagaattggaataaaattgattcattttaaaaaaaaa

SEQ ID NO: 32 - protein, 445 aa, Homo sapiens
MAEYDLTTRIAHFLDRHLVFPLLEFLSVKEIYNEKELLQGKLDLLSDTNMVDFAMDVYKNLYSDDIPHALREKRTTVVAQLKQLQAETEPIVKMFEDPETTRQMQSTRDGRMLFDYLADKHGFRQEYLDTLYRYAKFQYECGNYSGAAEYLYFFRVLVPATDRNALSSLWGKLASEILMQNWDAAMEDLTRLKETIDNNSVSSPLQSLQQRTWLIHWSLFVFFNHPKGRDNIIDLFLYQPQYLNAIQTMCPHILRYLTTAVITNKDVRKRRQVLKDLVKVIQQESYTYKDPITEFVECLYVNFDFDGAQKKLRECESVLVNDFFLVACLEDFIENARLFIFETFCRIHQCISINMLADKLNMTPEEAERWIVNLIRNARLDAKIDSKLGHVVMGNNAVSPYQQVIEKTKSLSFRSQMLAMNIEKKLNQNSRSEAPNWATQDSGFY

SEQ ID NO: 33 - DNA, 1133 bp, Homo sapiens
  CDS (133)..(942)
gcgcgggttggaggtgccacccggcgcgggtggcggagagatcagaagcctcttccccaagccgagccaacctcagcggggacccgggctcagggacgcggcggcggcggcggcgactgcagtggctggacgatggcagcgtccgccggagccggggcggtgattgcagccccagacagccggcgctggctgtggtcggtgctggcggcggcgcttgggctcttgacagctggagtatcagccttggaagtatatacgccaaaagaaatcttcgtggcaaatggtacacaagggaagctgacctgcaagttcaagtctactagtacgactggcgggttgacctcagtctcctggagcttccagccagagggggccgacactactgtgtcgtttttccactactcccaagggcaagtgtaccttgggaattatccaccatttaaagacagaatcagctgggctggagaccttgacaagaaagatgcatcaatcaacatagaaaatatgcagtttatacacaatggcacctatatctgtgatgtcaaaaaccctcctgacatcgttgtccagcctggacacattaggctctatgtcgtagaaaaagagaatttgcctgtgtttccagtttgggtagtggtgggcatagttactgctgtggtcctaggtctcactctgctcatcagcatgattctggctgtcctctatagaaggaaaaactctaaacgggattacactggctgcagtacatcagagagtttgtcaccagttaagcaggctcctcggaagtccccctccgacactgagggtcttgtaaagagtctgccttctggatctcaccagggcccagtcatatatgcacagttagaccactccggcggacatcacagtgacaagattaacaagtcagagtctgtggtgtatgcggatatccgaaagaattaagagaatacctagaacatatcctcagcaagaaacaaaaccaaactggactctcgtgcagaaaatgtagcccattaccacatgtagccttggagacccaggcaaggacaagtacacgtgtactcacagagggagagaaagatgtgtacaaaggatatgtataaatattctatttagtcatccttaaaaaaaaa

SEQ ID NO: 34 - protein, 269 aa, Homo sapiens
MAASAGAGAVIAAPDSRRWLWSVLAAALGLLTAGVSALEVYTPKEIFVANGTQGKLTCKFKSTSTTGGLTSVSWSFQPEGADTTVSFFHYSQGQVYLGNYPPFKDRISWAGDLDKKDASINIENMQFIHNGTYICDVKNPPDIVVQPGHIRLYVVEKENLPVFPVWVVVGIVTAVVLGLTLLISMILAVLYRRKNSKRDYTGCSTSESLSPVKQAPRKSPSDTEGLVKSLPSGSHQGPVIYAQLDHSGGHHSDKINKSESVVYADIRKN

SEQ ID NO: 35 - DNA, 1602 bp, Homo sapiens
  CDS (135)..(839)
ctcaggccggggcgcgaccgcggatccgcagttcccgggccagcctggggcggccggccaggaaccacccgttaaggtgtcttctctttagggatggtgaggttggaaaaaggctcctgtaaccctcctccaggatgaaccacctgccagaagacatggagaacgctctcaccgggagccagagctcccatgcttctctgcgcaatatccattccatcaaccccacacaactcatggccaggattgagtcctatgaaggaagggaaaagaaaggcatatctgatgtcaggaggactttctgtttgtttgtcacctttgacctcttattcgtaacattactgtggataatagagttaaatgtgaatggaggcattgagaacacattagagaaggaggtgatgcagtatgactactattcttcatattttgatatatttcttctggcagtttttcgatttaaagtgttaatacttgcatatgctgtgtgcagactgcgccattggtgggcaatagcgttgacaacggcagtgaccagtgcctttttactagcaaaagtgatcctttcgaagcttttctctcaaggggcttttggctatgtgctgcccatcatttcattcatccttgcctggattgagacgtggttcctggatttcaaagtgttacctcaagaagcagaagaagaaaacagactcctgatagttcaggatgcttcagagagggcagcacttatacctggtggtctttctgatggtcagttttattcccctcctgaatccgaagcaggatctgaagaagctgaagaaaaacaggacagtgagaaaccacttttagaactatgagtactacttttgttaaatgtgaaaaaccgtcacagaaagtcatcgaggcaaaaagaggcaggcagtggagtctccctgtcgacagtaaagttgaaatggtgacgtccactgctggctttattgaacagctaataaagatttatttattgtaatacctcacagacgttgcaccatatccatgcacatttagttgcctgcctgtggctggtaaggtaatgtcatgattcatcctctcttcagtgagactgagcctgatgtgttaacaaataggtgaagaaagtcttgtgctgtattcctaatcaaaagacttaatatattgaagtaacacttttttagtaagcaagatacctttttatttcaattcacagaatggaatttttttgtttcatgtctcagatttattttgtatttcttttttaacactctacatttcccttgttttttaactcatgcacatgtgctctttgtacagttttaaaaagtgtaataaaatctgacatgtcaatgtggctagttttatttttcttgttttgcattatgtgtatggcctgaagtgttggacttgcaaaaggggaagaaaggaattgcgaatacatgtaaaatgtcacgagacatttgtattatttttatcatgaaatcatgtttttctctgattgttctgaaatgttctaaatactcttattttgaatgcacaaaatgacttaaaccattcatatcatgtttcctttgcgttcagccaatttcaattaaaatgaactaaatt

SEQ ID NO: 36 - protein, 234 aa, Homo sapiens
MNHLPEDMENALTGSQSSHASLRNIHSINPTQLMARIESYEGREKKGISDVRRTFCLFVTFDLLFVTLLWIIELNVNGGIENTLEKEVMQYDYYSSYFDIFLLAVFRFKVLILAYAVCRLRHWWAIALTTAVTSAFLLAKVILSKLFSQGAFGYVLPIISFILAWIETWFLDFKVLPQEAEEENRLLIVQDASERAALIPGGLSDGQFYSPPESEAGSEEAEEKQDSEKPLLEL

SEQ ID NO: 37 - DNA, 1393 bp, Homo sapiens
  CDS (33)..(1208)
agacagcagaagggaaggatgtcacttctgagatgaggttcagaaaggcctgggctcctgtcctggctgctctctcccactccctgatgagcttgctggatgaaagctcctgtcaggctgtggggcgtcctgtggagaaactggcaagaaactggtgggggccctttccacctatagccagcaaggaactgaacccagccagcgtccacctgagtgagctcagaggagccaacatcaaaagcatttgttgttttgaggtgcgtctgcttctacgctttgcctgggagaggccctggtggcctcgttcctggcgcccggagtccctgctgcggccccacccccgggcggtcacggtgacccatgctgcccagcctggaggtaaaatcgttcgtggctgtggcttcagcatgtcgtcctcggtgaaaaccccagcactggaagagctcgttcctggctccgaagagaagccgaaaggcaggtcgcctctcagctggggctctctgtttggtcaccgaagtgagaagattgtttttgccaagagcgacggcggcacagatgagaacgtactgaccgtcaccatcacggagaccacggtcatcgagtcagacttgggtgtgtggagctcgcgggcgctgctctacctcacgctgtggttcttcttcagcttctgcacgctcttcctcaacaagtacatcctgtccctgctgggaggcgagcccagcatgctaggtgcggtgcagatgctgtccaccacggttatcgggtgtgtgaaaaccctcgttccttgctgtttatatcagcacaaggcccggctttcctacccacccaacttccttatgacgatgctgtttgtgggtctgatgaggtttgcaactgtggttttgggtttggtcagcctgaaaaatgtggcggtttcgtttgctgagacggtgaagagctccgcccccatcttcacggtgatcatgtctcggatgattctgggggagtacacaggacgtcccagtgatcgggaggagtgggaagagcttcagctacaaccaggacgtggtgctgctgcttctgacagacggagtcctgttccaccttcagagcgtcacggcgtacgccctcatggggaaaatctccccggtgactttcaggtcccgcaggccctgcaccgagtcgccttgtccatggcgctgccctgccccatgcttcctgcgtcctgagtaggaggtatctccgagacaggaaaagtggctgctctctctgactttttctggaactcatggtggtctcctgggcttggtcactgtctctcaccagcatgtttctttgtgcggtcaggaattatttccaaatgctcctgaagcctagtgttttagtgaacattagtgattgttagcagtggttc

SEQ ID NO: 38 - protein, 391 aa, Homo sapiens
MRFRKAWAPVLAALSHSLMSLLDESSCQAVGRPVEKLARNWWGPFPPIASKELNPASVHLSELRGANIKSICCFEVRLLLRFAWERPWWPRSWRPESLLRPHPRAVTVTHAAQPGGKIVRGCGFSMSSSVKTPALEELVPGSEEKPKGRSPLSWGSLFGHRSEKIVFAKSDGGTDENVLTVTITETTVIESDLGVWSSRALLYLTLWFFFSFCTLFLNKYILSLLGGEPSMLGAVQMLSTTVIGCVKTLVPCCLYQHKARLSYPPNFLMTMLFVGLMRFATVVLGLVSLKNVAVSFAETVKSSAPIFTVIMSRMILGEYTGRPSDREEWEELQLQPGRGAAASDRRSPVPPSERHGVRPHGENLPGDFQVPQALHRVALSMALPCPMLPAS

SEQ ID NO: 39 - DNA, 5803 bp, Homo sapiens
  CDS (2)..(334)
gatgaatgcaatggtacctcacatttcaatactaacattagatgtaaatggcctaaatgctccacttaaaagatgcagagctgcagaatggataaaaagtcacgaaccaaccatctgctgccttcaggagactcacctaacatataaggcctcacataagtttaaagtaaaggggtggaaaaaggcatttcatgcaaatggacaccaaaagcgagcaggagtagctattttatcagacaaaacaaacttgaaagcaacagcacttaaaagagatgaagagggacattacataatggcaaaagccttgtccaacaggaaaatatcacaatcctaaacaagaccagacaattatcccatcatcagtgttgtgctcagtacatggactaggtagcagaaatcagtgatagcttttattacagcatctcacttaaggtttttcattgaatatagtattcccttggagttgatgggcatatgacttaattggtttcaattggtcattgaattccactgattacgaagtgatccaatatattttttattttgtatataaacctgtggggcagcactgaaaaagaacttatgattctcagtgtttaaaataagatctggagtcagataaacctgtttttgaatcttggctcttctactcctatttgggcaatcataggcaaattataatgtctgtaagttccatgtttctcatctttaaaatgggtattaaaaatagcttgcaggattatcctgaggattaattaacataaaacagaaatgcactaagcttctaacatgatgcatagtaaaacacttaatatctttggttgggtccccaggagcagactccaagacaaggatttgggtgcaagtagtttgagaggttgttatagactgatgtgtccctttcaaaaaactgatatgttgaaatcttaatccccaatgtgatggtattagaaggtaggaactttgggaggtaatttaggtgatgagggtagagtcttcatgaatgggattagtgcccttacaaaagacaacttagagggctctctaactctttctgtcatgtgaggacacaatgagaagttgacactctgcaacttggaagggagccctcacaatgctggcatcctgatcgcagactctcagtatccagaactttgagaagtaaatttctgttgtttataagtcatgcagtctatggtaatttgtcatagcagcccaatataagacagaggtcaccccaggaaatactggtagtagatggagaagtcagacaagaaagagaagaaagccaacaaggagtggattaccacacacatttgcattgtgagtaattggagcttaatcttactggggaactctgagaaccaatgtggaagatagacctcagaggtatctccctagaagacagaagaaactgaggtattaatctaccaactcctatcagtcatcaattcttaattctctggcactacaaccttccatgtatatgagcagagaaggatccagtgaccagaacaagcccttaaggaaagtgatgtaggtgatacagttggaagatgggccagcatatgcaaaaatcgttcagggaaaaagggacataggcaggcactgatatctatagtagctcaataaacacttgatagcagtagctatatatgggagggttttcatgccagaagcttgtttacacatcaactactttgatagaggtgggggagaagagttaaagcagcattagttcatctcctaaggtttgtcccagctctaatatttacaaatatctcatggtgtatttctttctgtatgcacaagcactgctctttttggtgaattaaaatcacaagaaacccttatgcagttttacttttaataatgggaaacggtactgttgtaaccattaaattctaatatgataaaagataagtttatctagaggcagtgcagttcagtgtaaagaccacaggatgaagagttaggagatctttgtggagtctttactcagtcacttaccatttgtgtggcctcttaaaggagtaaaaatctacaagtctcaactgaaaggaaaacattacctgcctagctaatgtgttatagtgagagcacatggaacaatgctttgaagaggagttgtctcagatgctgagctctacataggagcagtcaagttttctaccagtcagaagggcaaagtgcttctggttgcttttggcaggagtactgaattcactgtaaggcttgatgattaccttgtgcttctcccatatccacagcactgaagtaatttgtgatttgaagacacagtgactctcttggggcacattagacacatagatgtaaatcaattgattccaaggattctgtgcagctgttgtcttcgatggatatagtggctgcaactgcctgtttcagtttcctcctggaaaactcttgatatcagtctctgacatcttggagtatttctttggctccacagacattaaggaaaatcatctatagaaaatatttatataattttatctttgcttgttctcaaatccaagggatgatcatgccaaaattttaggagtgaaatatgtctttgcccacagtgtgcctaccactttataaattatacccttatacaaagtattggttttgaattttggaactaattataactaggtaaatgcttaactggaagactttggaatttcatgaagtcagtgcctttaaagttatgcactttaaagagatctttttgtattaattaggatatccagtaagtctttttgtgcagctccacgtcgtagcaaggaatttataatatgtatgtattttaaagatgcatgtggcattctagcaattacacagtctcagtgtgatataagtgtaaattagtcttaccagattctgtatttaaaattgtagtgtaaaactatatacattttaagggtgaatcctactgttagagaaaaataattgcatcaattttcaataaactttgacaaatatggaattaatctaagttaactactaagaccctaaacttaagtacctgagaacttagatatttacaaggtaatggttattaatcagaaagcctaaaacttataaaaatatcattcagtattagtgttcttatagtattttctagtttcaatttttagacaaaaattttggtaacataatttggaaaataaaaatgatgaagtaattaaatgtactaaaagataattgtctcatttttttttctttaaagattttgtttgctcatgtcaatgttatctatcactttaatagaagcataaactttagagcatccatgatgataatgtaggcagaatatttttagatagtatttcatatatatgtaatttattgtaagccaatttaaagtttccttattataagccaatttaaagtatttcatatatatgtaatttattataagccaatttaaagtttccaattaaagtgaaatatatttcacacacataataaaataaatcttgatctttctaaaaaaatcaatatgtactttcttatgtgtaaacaaaaaaattcatgcaagtaaatcacaataatataccaccaatgactctggcaataaagcacacaaataataaagtaaaagttatgaccttccattaaccgtaaacactaatcctggtaaaggggatgagaaattgtttataataactcagcacataataactgcatttgagaaaccaattactgcttttatatggcttcagtttctcagtctagcacttcatttttgcaataaaggacctgagtacatttgcatccagtgcacaaaaggtgtgcaatgaataactgcagtttaattgggctcttaagaattctgatatcttcattaacttatttcatgacaaatgtatttatatctttgcaactgtttttactgaattatatacataaattttttaaccagtttaaaatggctcatctgttaagagatctgaatgcataaagttttagtagtaagacaatttatcttgataaatgaagtcaagcaatgtagatttagcaaggttccagagctttcagtgatcatacagtggacctttaaagaaacataaaaagaagaggttcgatcataatggcagaagggaggcaggactagattgcagctctggacagagcagtgtgcgggggctcatattgtgagttttagctccaaattgactgcaagaacaaaccagcgatccccagaggacccacagaccctctgaagaaaggggtctgctcctgcaggacctgggagacatccccaaaactgtgagtgatccaagtgggaaagggagaccctcctctcccaaacacacactcctactggagaagctgaaggtctgtttgtgggagaagtttccgactttacttggagctgagtcaatttggagagctgagtgaaatacaggggtagaggaagcagcagaaaggccctgggagcttgctgggtctcctagcaggccactcctgcctggcaccacagtgatccaacgcgagatgagcactgggtaaaactacacggggagaagcatatctatagctcaactttgtaacaatttgaactggatgagaagccttctggccagaactcgggggagggcacaaatctggtgtgcaggctccacaggcaggggaagaaccaagccctttggagtgagactggctcttcagtttgtgtgggagctgggtgaagcctgtgactgccagctttcccccacttccctgacaacctgcgtgactcagaagaggcggccataatccgcctacatacacaactccagtgacttgggaatctcacccccattccccacagcagctgcaggaagacctgcccaaggagagtctgagctcaaacatgcctagccccacccccacctgatggtccttccctacccaccctggtagcagaagacaaagggcatataatcttgggagttctagggccccacccaccaccggttcctccccattctaccacagctgatgctctctggaaagcgccacctcctgacaggaggccaaccagcacaaaaataaagcattgaaccaccaaagctaagaaccctcacagagtccattgcatcccccaccacctccactggaacaggctctggtatccaccactgagagacccatagacggttcacatcacaaaactctgtgcagacaacccccagtaccagctcaaaactgggtagacttgctgggtggctagacccagaagagagacaacaatcactgcggtttggctcacaggaagccgcatccataggaaaagggggagagtactacaacaagggaacatcatgtgggacaaaagaatctgaagaacagccttcatccctagaccttccctctgacagagcctacccaaatgagaaggaaccagaaaaccgtggtaatatgacaaaacaagactcatgaacacccccaaaaatcacactagttcaccagcaatggaaccaaaccaagaagaaatccctgattaacctgaaaaagaattcaggaggttagttattaagctaatcagggagggaccagagacaggtgaagcccagtgcaaggagatccaaaaaatgatacaagaagtgaagggagaaatattcaatgaaatagataggttaaagaaaaaacaatacaaaattcaggaactttgagcacacttttagaaatgtgaaatgctctggaaagtctcagcaatagaattgaacaagtagaagaaaaaaatttagagctcaaagacaaggtcttcaaattaacccaatccaacaaagacaaaaaaaaaaaaaaa

SEQ ID NO: 40 - protein, 110 aa, Homo sapiens
MNAMVPHISILTLDVNGLNAPLKRCRAAEWIKSHEPTICCLQETHLTYKASHKFKVKGWKKAFHANGHQKRAGVAILSDKTNLKATALKRDEEGHYIMAKALSNRKISQS

SEQ ID NO: 41 - DNA, 1764 bp, Homo sapiens
  CDS (697)..(1179)
ctcttacagcgcacccgttggtgcgcgggaataggtgtgcatgccccggcctgggcctttttctgttgacccacggcatcaccttagcaagggtgttgtccttttcagtccattccctgaagcgcagaaccggaggccttgtgagaacctggctttttgtccagtcctgtcctcagaactcaaggaggcatcacggggggagtcatttacctccctggtctcaggtgtctctcacagtacactcctgatcatgtagttggacctggagcagacattgatcccactcaaataacctttcccggatgcatttgtgtcaaaactccctgcctccctggcacttgctcctgtctccgccatggagagaactatgatgataactcatgccttagagatataggatctggaggaaagtatgcagagcctgtttttgaatgcaatgtcctgtgccgatgcagtgaccactgcagaaacagagtggtccagaaaggtctacagttccacttccaagtgttcaagacgcataaaaaaggctggggacttcgtaccttggaatttataccgaaaggaaggtttgtctgtgaatatgctggtgaggttttaggattctctgaagttcagagaagaattcacttacaaacaaaatccgactccaattacattatagccatcagggaacatgtttataatgggcaggtaatggaaacatttgttgaccctacttatataggaaatattggaagattccttaatcattcttgtgagccaaaccttttgatgattcctgtccgaattgactcaatggtacctaagttggcactttttgcagccaaagatattgtgccagaagaagaactctcttatgattattcaggaagatatcttaatctaacagtcagtgaagacaaagaaaggctagatcatgggaaactaaggaaaccttgttactgtggtgccaaatcatgtactgctttcctgccttttgacagttctctgtactgccccgtagaaaagtcgaacatcagttgtggaaatgagaaggaacccagcatgtgtggctcagccccttctgtgttcccctcctgcaagcgattgacccttgaggtgagtctgttcagtgataagcagcttgcccctccctatagtggaagacagtggttggctagctttacctctgcctagttacataagtttaactcaggaatgtggcagagactgcaagttgtccatcagtatcctttcccctttctgtaatagaattctccatttaacaaaagtacatgactacccagaatactcactttcctagttagatttgtcttagtgactgttctagttaataggatataagcaaaaatgacaagtccttaaatggagatatcctgcccttcacctcctctttcttcctgctggctggaatgtatacatgatgactggaactggagcagtcatcctagaccccaggatgaccctgggaaaggaagccattgcatagaaaaatcaacaaggttaaaaggagtctaggtgtctggtactgtagaacatcctgtcagtcgtgatctgacacctttgtactttcaggtgagaaatagtctctcccttgcttaagtcactgttattgtaagttttctagcactaacatcgaacctaataatgatccaagaaacccttgtggctgcatacctaaaccctgcttgccaaccaattttaacagtaataaagctgactttttgatttcaaaaaaaaaaaaaaaaaaaaaaaaaaaaa

SEQ ID NO: 42 - protein, 160 aa, Homo sapiens
METFVDPTYIGNIGRFLNHSCEPNLLMIPVRIDSMVPKLALFAAKDIVPEEELSYDYSGRYLNLTVSEDKERLDHGKLRKPCYCGAKSCTAFLPFDSSLYCPVEKSNISCGNEKEPSMCGSAPSVFPSCKRLTLEVSLFSDKQLAPPYSGRQWLASFTSA

SEQ ID NO: 43 - DNA, 1042 bp, Homo sapiens
  CDS (283)..(744)
atagcggtttgactcacggggatttccaagtctcccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgcgaattcggcacgagaggggtcggaggtcagggcgagcgtctcgcaggccgtaggaggaagatggcggtggagtcgcgcgttacccaggaggaaattaagaaggagccagagaaaccgatcgaccgcgagaagacatgcccactgttgctacgggtcttcaccaccaataacggccgccaccaccgaatggacgagttctcccggggaaatgtaccgtccagcgagttgcagatctacacttggatggatgcaaccttgaaagaactgacaagcttagtaaaagaagtctacccagaagctagaaagaagggcactcacttcaattttgcaatcgtttttacagatgttaaaagacctggctatcgagttaaggagattggcagcaccatgtctggcagaaaggggactgatgattccatgaccctgcagtcgcagaagttccagataggagattacttggacatagcaattacccctccaaatcgggcaccacctccttcagggcgcatgagaccatattaaattctatttactatttgttgaatttatttttccgtcagttatgtaaaataaacatactcttcttcctcccctgattattgccattaagcctttaaattctaaacaaattataatgcatcatctatttaggagttagatttggatgtgctattgtatgattacgaatagtctgtatgtttcaagcccttctgtaaaatatgaagaaaagtgctcttagcattctgtgtaaaactgtactgttaaatatatgtgtgtaatcagccaaaaaaaaaaaaaaaaaaaaaaaaaaaaaaaaaaa

SEQ ID NO: 44 - protein, 153 aa, Homo sapiens
MAVESRVTQEEIKKEPEKPIDREKTCPLLLRVFTTNNGRHHRMDEFSRGNVPSSELQIYTWMDATLKELTSLVKEVYPEARKKGTHFNFAIVFTDVKRPGYRVKEIGSTMSGRKGTDDSMTLQSQKFQIGDYLDIAITPPNRAPPPSGRMRPY

SEQ ID NO: 45 - DNA, 1523 bp, Homo sapiens
  CDS (758)..(1330)
catccttatctctcacttttgtgtgagttttgggatcagggtttccagttttatttggtgcccaaagtgtttcttgggttggacctcgcttcttcctctaagctcacagctacaacaaaactctcaataataattctttttcttttcttttttttgagacactgtctcgctctggcgcccaggctggagtgcagtggcgcaatctcggctcactgcaacctccgcctcccgtgttcaagcgattttcccgcctcagctgggattacaggcacgtgccaccatgcccggctaattttttgtatttttagtagagacaggggtttcaccatgttagccaggctggtctcaaactcctgacctcaggtgatccgcccgcctcatcctcccaaagtgctgggatgacaggcctgagtcaccgtgcccggcccctgactagaaatatcttatttgcttgctcgttgcctctctacagtaggatatgtgctcagggagagctatatccccagtctgagaatgatccctgatactgtgacactagaagcacttgttgaataaacagtgctatcaagagtttctatcacattgcattgcagttttatatccccaaaggtcgtggattggtgaacagggctgccatttttgtaatggatgccaatgcctgacagaaattgctgtatgaatgattccaacttccagcagctaaattatttatggcacacaaaaacattcttttcaggtcctgcatctccagtatggaatgtatgtttggccctgtgctgtggtcctggcccagtacctttggtttcacagaagatctctgccaggcaaggccatcttagagattggagctggagtgagccttccaggaattttggctgccaaatgtggtgcagaagtaatactgtcagacagctcagaactgcctcactgtctggaagtctgtcggcaaagctgccaaatgaataacctgccacatctgcaggtggtaggactaacatggggtcatatatcttgggatcttctggctctaccaccacaagatattatccttgcatctgatgtgttctttgaaccagaagattttgaagacattttggctacaatatattttttgatgcacaagaatcccaaggtccaattgtggtctacttatcaagttaggagtgctgactggtcacttgaagctttactctacaaatgggatatgaaatgtgtccacattcctcttgagtcttttgatgcagacaaagaagatatagcagaatctacccttccaggaagacatacagttgaaatgctggtcatttcctttgcaaaggacagtctctgaattatacctacaacctgttctgggacagtatcaatactgatgagcaacctggcacacaaactatgagcagaccacttcagcttgagaatgcagtgggtctgaagatggtcaagtctgtttgccttagattttgatgtcacctagacaacacttaaactcatatgaaacaaaaattaaaatacgtattacaagt

SEQ ID NO: 46 - protein, 190 aa, Homo sapiens
MYVWPCAVVLAQYLWFHRRSLPGKAILEIGAGVSLPGILAAKCGAEVILSDSSELPHCLEVCRQSCQMNNLPHLQVVGLTWGHISWDLLALPPQDIILASDVFFEPEDFEDILATIYFLMHKNPKVQLWSTYQVRSADWSLEALLYKWDMKCVHIPLESFDADKEDIAESTLPGRHTVEMLVISFAKDSL

SEQ ID NO: 47 - DNA, 1454 bp, Homo sapiens
  CDS (234)..(971)
cagccccgttgttgccctccgcgtatcccctcaccacctttgcggccatccacgactttcgcaccttccgccattttcctgcctgtgagggtggacagatcgcgctcgggtctcggcctcctgagtgccggtgactgcgggaggcgacggagtgcttctgggggtgtgagctggggaagttcgtggtcacggatgcgtgtggggttgctgctcagtctgtaacggcaggaaagatgaatgggagggctgattttcgagagccgaatgcagaggttccaagaccaattccccacatagggcctgattacattccaacagaggaagaaaggagagtcttcgcagaatgcaatgatgaaagcttctggttcagatctgtgcctttggctgcaacaagtatgttgattactcaaggattaattagtaaaggaatactttcaagtcatcccaaatatggttccatccctaaacttatacttgcttgtatcatgggatactttgctggaaaactttcttatgtgaaaacttgccaagagaaattcaagaaacttgaaaattccccccttggagaagctttacgatcaggacaagcacgacgatcttcaccacctgggcactattatcaaaagtcaaaatatgactcaagtgtgagtggtcaatcatcttttgtgacatccccagcagcagacaacatagaaatgcttcctcattatgagccaattccattcagttcttctatgaatgaatctgctcccactggtattactgatcatattgtccaaggacctgatcccaaccttgaagaaagtcctaaaagaaaaaatattacatatgaggaattaaggaataagaacagagagtcatatgaagtatctttaacacaaaagactgacccctcagtcaggcctatgcatgaaagagtgccaaaaaaagaagtcaaagtaaacaagtatggagatacttgggatgagtgaaaaattacatcattggacatgaaggagtttcaacatccagcttcatctaggtggtcatgattacctgcatgctttgagctcagcagcagtcttcataaacacatttaaaacaagatcctgggtttttgtggtttgacttctatggtgttttaaaaaaacacagatttttagtgttaatattgtgtaaatgtactcaccttagggattcatttgaatgatggtattataccatgattgtatacagtttgtgaaattgttgcaagggcaaagataactcttaaaaaaccgtcgagattacaatgctctagaatcagcatataagaaaataaatgatatctgcatgttgaattggggtggatggggggagcaagcataatttttaagtgtgaagctttgcatcaagaaattattaaaaagctttttttctccagtattttctgtattatcttaatgtttatggcaaataaaatgtaaaggaacatg

SEQ ID NO: 48 - protein, 245 aa, Homo sapiens
MNGRADFREPNAEVPRPIPHIGPDYIPTEEERRVFAECNDESFWFRSVPLAATSMLITQGLISKGILSSHPKYGSIPKLILACIMGYFAGKLSYVKTCQEKFKKLENSPLGEALRSGQARRSSPPGHYYQKSKYDSSVSGQSSFVTSPAADNIEMLPHYEPIPFSSSMNESAPTGITDHIVQGPDPNLEESPKRKNITYEELRNKNRESYEVSLTQKTDPSVRPMHERVPKKEVKVNKYGDTWDE

SEQ ID NO: 49 - DNA, 1414 bp, Homo sapiens
  CDS (81)..(905)
ctgggtcacagagcctaaaacgacacacccaacacgcccgccggagttacagctaaaggaaggacaggggaagcaatgaaatgccgagggcggagccaagagcgacactgggggagcaggaaaaggcggggcttccgcttggggcatggaggctgtacctcttacgtcacttccgtaaacaaacggagctgcggaggagcgggtcccgggatgtgaccggggctctgcttgtggctgcggcggtggcttctgaggctgtcgggtctttgcgggttgcggaagggggccccaatacccttcttcttcaggtcttaagaagctggccgtggtgcaataaggaacttaaaacaatggaagagcggaaagtgaagaggaggagtcctaagtcttttagtgcccactgtactcaggttgtcaatgccaaaaaaaatgccattccagtgagtaaaagcacagggttttcaaatcctgcatcacagtcaacttcacagcgaccaaagttaaaaagagtgatgaaagaaaagaccaaacctcagggtggagagggcaaaggcgctcagtcaactccgatccagcactccttcctcactgatgtctcagatgttcaggagatggagagagggctgctcagtcttttgaatgatttccactctggaaaacttcaagcatttggaaatgaatgttccattgaacagatggaacatgttcggggaatgcaggagaaattagctcgcttgaatttggagctctatggggagttagaggaacttcctgaggataagagaaaaacagccagtgactccaatctggataggcttctgtcagatttagaagaattgaattcttccatacaaaaactccatttggcagatgcacaagatgttccaaatacttctgctagctaaaatgaaatgtagtttgctttcttgtgatttgaagagaagcagcagtctttacttttccagccaaatccagtagcagaatcatcttccacaacaaggaattaaagtaattaaagtgcaagttcaatgatcgttttcacttactgcttttagtaaatgtgactcgctgtaattcgccataactggaggcctaggccttgttgaaggcttcatcaggaaatgtgacgcagagattgtgctgctgtgcttcatattgttgccttatgggattatacttgaaatgcattgtgctgatgttcttgacagggcggagggatttctctttcctgagctcaccaaacttattccagtgttgatcgcaagctgttgatgcacaggcgtcttgtggcaagcctcagcttcagtttatgttagacaatcagtacacatgctggtgtgttttccaatggccagtgataagaaattaaataagtatttgtgagatttgctattttttaataaagtaattgttt

SEQ ID NO: 50 - protein, 274 aa, Homo sapiens
MPRAEPRATLGEQEKAGLPLGAWRLYLLRHFRKQTELRRSGSRDVTGALLVAAAVASEAVGSLRVAEGGPNTLLLQVLRSWPWCNKELKTMEERKVKRRSPKSFSAHCTQVVNAKKNAIPVSKSTGFSNPASQSTSQRPKLKRVMKEKTKPQGGEGKGAQSTPIQHSFLTDVSDVQEMERGLLSLLNDFHSGKLQAFGNECSIEQMEHVRGMQEKLARLNLELYGELEELPEDKRKTASDSNLDRLLSDLEELNSSIQKLHLADAQDVPNTSAS

SEQ ID NO: 51 - DNA, 2115 bp, Homo sapiens
  CDS (60)..(1664)
ggcgctttgtgttctcatcggagctgcatgggaagtctgcgtacagcaaagtgacctgcatgcctcaccttatggaaaggatggtgggctctggcctcctgtggctggccttggtctcctgcattctgacccaggcatctgcagtgcagcgaggttatggaaaccccattgaagccagttcgtatgggctggacctggactgcggagctcctggcaccccagaggctcatgtctgttttgacccctgtcagaattacaccctcctggatgaacccttccgaagcacagagaactcagcagggtcccaggggtgcgataaaaacatgagcggctggtaccgctttgtaggggaaggaggagtaaggatgtcggagacctgtgtccaggtgcaccgatgccagacagacgctcccatgtggctgaatgggacccaccctgcccttggggatggcatcaccaaccacactgcctgtgcccattggagtggcaactgctgtttctggaaaacagaggtgctggtgaaggcctgcccaggcgggtaccatgtgtaccggttggaaggcactccctggtgtaatctgagatactgcacagacccatccactgtggaggacaagtgtgagaaggcctgccgccccgaggaggagtgccttgccctcaacagcacctggggctgtttctgcagacaggacctcaatagttctgatgtccacagtttgcagcctcagctagactgtgggcccagggagatcaaggtgaaggtggacaaatgtttgctgggaggcctgggtttgggggaggaggtcattgcctacctgcgagacccaaactgcagcagcatcttgcagacagaggagaggaactgggtatctgtgaccagccccgtccaggctagtgcctgcaggaacattctggagagaaatcaaacccatgccatctacaaaaacaccctctccttggtcaatgatttcatcatcagagacaccatcctcaacatcaacttccaatgtgcctacccactggacatgaaagtcagcctccaagctgccttgcagcccattgtaagttccctgaacgtcagtgtggacgggaatggagagttcattgtcaggatggccctcttccaagaccagaactacacgaatccttacgaaggggatgcagttgaactgtctgttgagtccgtgctgtatgtgggtgccatcttggaacaaggggacacctcccggtttaacctggtgttgaggaactgctatgccacccccactgaagacaaggctgaccttgtgaagtatttcatcatcagaaacagctgctcaaatcagcgtgattccaccatccacgtggaggagaatgggcagtcctcggaaagccggttctcagttcagatgttcatgtttgctggacattatgacctagttttcctgcattgtgagattcatctctgtgattctcttaatgaacagtgccagccttcttgctcaagaagtcaagtccgcagtgaagtaccggccatcgacctagcccgggttctagatttggggcccatcactcggagaggtgcacagtctcccggtgtcatgaatggaacccctagcactgcagggttcctggtggcctggcctatggtcctcctgactgtcctcctggcttggctgttctgagagctccgctgagcatctggccttgaagtttgtgttcttccctctggcaatggctcccttcagcacttctgctttccactccaattcacacaggcttggtattaacagaatcaaggccaggctaggttaggaaaagggaagagctttcaccttctttggggctctcggctgggcgcagtggctcatgcctgtaatcccagcattttgggaggctgaggcaggtggatcacctgaggtcagcagttcaaaatcagcctggccaaaatgctgaaactccgtctctactaaaaatacaaaaattagccaggcatggtggcaggcgcctgtaatcccagctactcgggaggccaaggcaggagaattgctcgaactcagggggtggaggttgcagtgagttgagattgtgccattgcactccagcctgggcaacagagcaagactctgtctcagg

SEQ ID NO: 52 - protein, 534 aa, Homo sapiens
MPHLMERMVGSGLLWLALVSCILTQASAVQRGYGNPIEASSYGLDLDCGAPGTPEAHVCFDPCQNYTLLDEPFRSTENSAGSQGCDKNMSGWYRFVGEGGVRMSETCVQVHRCQTDAPMWLNGTHPALGDGITNHTACAHWSGNCCFWKTEVLVKACPGGYHVYRLEGTPWCNLRYCTDPSTVEDKCEKACRPEEECLALNSTWGCFCRQDLNSSDVHSLQPQLDCGPREIKVKVDKCLLGGLGLGEEVIAYLRDPNCSSILQTEERNWVSVTSPVQASACRNILERNQTHAIYKNTLSLVNDFIIRDTILNINFQCAYPLDMKVSLQAALQPIVSSLNVSVDGNGEFIVRMALFQDQNYTNPYEGDAVELSVESVLYVGAILEQGDTSRFNLVLRNCYATPTEDKADLVKYFIIRNSCSNQRDSTIHVEENGQSSESRFSVQMFMFAGHYDLVFLHCEIHLCDSLNEQCQPSCSRSQVRSEVPAIDLARVLDLGPITRRGAQSPGVMNGTPSTAGFLVAWPMVLLTVLLAWLF

SEQ ID NO: 53 - DNA, 2262 bp, Homo sapiens
  CDS (119)..(1759)
aaaagaagaagaaaggaaaaaaaaagaaacagaccagaagaaggaagctgttgctcctgtgcaagaagaatcagatcttgaaaaaaaaaggagagaagctgaagcattgcttcaaagcatggggctaactccagaatcccccattgtccctcctcctatgtctccatcctccaaatctgtgagcactccaagtgaagctggaagccaagactctggagatggcgccgtgggatctagacgaggacctattaaacttggaatggctaaaatcacgcaagtcgactttcctcctcgagaaattgtcacgtatacaaaggaaactcagactccagttatggctcaacccaaagaagatgaagaggaagatgatgatgtagtggctcctaaaccacctattgaacctgaagaagagaaaactttaaagaaagatgaggaaaatgatagtaaagctccccctcatgagctgactgaagaagaaaagcaacaaatcttgcactctgaggaatttttaagtttctttgaccattctacaagaattgtagaaagagctctttctgagcagattaacatcttctttgactatagtgggagagatttggaagacaaagaaggagagattcaagcaggtgctaaactgtcattaaatcgacaattttttgacgaacgttggtcaaagcatcgggtggttagttgtttggattggtcatctcagtatccggagttactcgtggcttcctataacaacaatgaagatgcccctcatgagcctgatggtgtggcccttgtatggaatatgaaatacaaaaaaactaccccagagtatgtgtttcactgccagtcagctgtgatgtctgccacatttgcaaaatttcatccaaatcttgttgttggtggtacatattcaggccaaattgtgctttgggataaccgtagcaataaaagaactccagtgcaaagaactccactgtcagcagctgcacacacacaccctgtatattgtgtaaatgttgttggaacacaaaatgctcacaatctgattagcatctctactgatggaaaaatttgttcatggagtctggacatgctttcccatccacaggatagcatggagttggttcataaacagtcaaaagcagtagctgtgacatctatgtccttccctgttggagatgtcaacaactttgttgttgggagtgaagaaggttctgtgtacacagcatgccgccatggcagcaaagctggaatcagtgagatgtttgaggggcatcaaggaccaatcactggcatccattgtcatgcagctgttggagcagtagacttctcacatctttttgtcacttcatcgtttgactggacagtaaagctttggacaactaagaataacaagcctttgtattcatttgaagataatgcagactatgtttatgatgttatgtggtcacctacccacccagccctgtttgcctgtgtggatggcatggggagattggatttgtggaatctcaataatgacacagaggtaccaactgccagcatttctgtggagggtaatcctgctcttaatcgtgtgagatggacccattctggcagagagattgctgtgggtgattctgaaggacagattgttatatacgatgtgggagagcagattgctgttccccgcaatgatgaatgggcacggtttggccgaacacttgcagaaattaatgcaaaccgagctgatgcagaggaggaagcagctacccgaatacctgcttagttcctgaaaaggggagtgtaactagtggatttgggaaaggttcttaagtagatcctgagactatttgcatgcttctgtctaaatgataattaaaaggaaatttcatggattaaaccatgggtttaatgcagcaaggaaacttacaatgtccctttatatataacatgcatcttgttttggatttgtgtcattttttaatatagctgattgacttcacagaaagcagcttttttgaattctaatacataggtgtatatttggtattagttattttgagttcttttcaacttataacactgtatacagttatttctaaagcacagatgaaataagttctgcatatttttaaataatcacagttccctgttatacagataatgttctcactacccataatatgtaggaacattgtttctccttagccgtagtatgcatacacctatccatgttcattctgacatcctttgttgtctttataattcatgtggtagttacctataaat

SEQ ID NO: 54 - protein, 546 aa, Homo sapiens [sequence omitted]

SEQ ID NO: 55 - DNA, 4310 bp, Homo sapiens
  CDS (87)..(3644)
ccgctgtaacctcttcggtccgcgacgatcctctagagcactgtgtgtctccccggacgcgagcccgctcccctgaaatctgcaaaatggctgataatttggatgaatttattgaagagcaaaaagccagattggccgaagacaaagcagagttggaaagtgatccaccttacatggaaatgaagggaaagttgtcagcgaagctttctgaaaacagtaagatactgatctctatggctaaggaaaacataccaccaaatagtcaacagaccaggggttccttaggaattgattatggattaagtttaccacttggagaagactatgaacggaagaaacataaattaaaagaagaattgcggcaagattacagacgttatcttactcaggaaaggttgaaacttgaacgtaacaaagaatacaatcagtttctcaggggtaaggaagaatccagtgaaaagttcaggcaggtggaaaagagtactgagcccaagagtcagagaaataaaaaacctattggtcaagttaagcctgatctaacttcacaaatacagacatcttgtgaaaattcagagggtcctagaaaagatgtcttaactccttcagaggcatatgaagaacttctgaaccaaagacgactagaggaggacagataccgacaactagatgatgaaatcgaattaaggaatagaagaattattaaaaaagcaaatgaagaagtgggcatttccaacctaaaacatcaaaggtttgcaagcaaggctggcattccagatagaagatttcacagatttaatgaggatcgtgtttttgatagacggtatcatagaccagaccaagatcctgaagtaagtgaagaaatggatgagaggtttagatatgaaagtgattttgatagaagactttcgagagtgtatacaaatgacaggatgcacaggaacaaacgagggaatatgcctcctatggaacatgatggggatgttatagaacagtcaaacataagaatttcatctgctgaaaataaaagtgctccagacaatgaaacatccaaatctgctaatcaagatacctgtagtccttttgcagggatgctctttggaggtgaagatcgagaacttattcagagaaggaaagagaaatacagactagaactgttggaacaaatggctgagcaacagaggaacaagagacgagaaaaagatttagaactcagggttgcagcgtctggagcacaagaccctgagaaatcgcctgatagactaaagcagtttagtgtggcaccaagacactttgaagagatgataccacctgaaagacccagaatagctttccagacacctctccctcctttatctgccccatctgtcccacccatcccatcagttcatcctgttccttctcaaaatgaagatttgcgcagtggactcagcagcgcccttggtgaaatggtgtctcccaggattgcacctctgcctccacctcccctactaccacctttggctactaactatcgaactccttatgatgatgcatactatttttatgggtccaggaatactttcgatcccagtcttgcttattatggttcaggaatgatgggcgtacagcctgcagcttatgttagtgctcctgtcacccaccaactagcacaacctgttgtgaatacggttggacagaatgaactgaagattacaagtgatcaagtgataaattcaggattgatttttgaagataaaccgaaaccttccaaacagtcacttcagtcttaccaagaggctttgcagcagcagattcgggaaagagaagaaagaaggaagaaagaacgtgaagaaaaagaagaatatgaagctaaattagaagctgaaatgagaacatataatccctggggaaaaggtggaggtggtgctcctctcagggatgcaaaaggaaatctgataactgatttgaataggatgcacagacaaaatatagatgcctaccataacccagatgcaagaacatatgaagataaaagggctgttgtatctctagacccaaatttagccacttcaaatgctgagaacctagaagatgctgcaaataaaagctcaggtcatatgcaaacacagagctctccttttgctcggggaaatgtatttggtgggcctccaactgaacttcagattaaacagcaagaattatacaagaattttcttcgtttccagattgaggaaaagaaacaaagagaggaagcagagcgagagagactgagaattgcagaagaaaaagaagaaagacggcttgcagaacagagggcacgaattcagcaggagtatgaagaggaacaggaaaagaaaagagagaaagaggaggagcaaaggctaaaaaatgaagagcatattcggttagctgaagaaagacaaaaagaagcagaaagaaagaagaaagaagaagaagaaaaatataacctgcaacttcagcactactgtgaaagagacaatttgattggggaagaaacaaagcacatgagacagccttctcctatagttcctgctcttcagaacaaaattgcaagcaaactccaaagacctccttcagttgacagcatcatacgttcctttattcatgaaagttccatgtccagggcacagtcacccccggtacctgccaggaaaaatcagctccgtgcagaagaggagaaaaaaaatgtaattatggaattatcagaaatgagaaaacagcttcgtagtgaagagaggcgtctacaagagcgattgctacacatggacagtgatgatgaaattcctatcaggaaaaaggaaaggaatcccatggatatatttgatatggctagacatcggttgcaagctcctgtcagaagacagtcccctaagggcttagacgctgccacttttcagaatgttcatgattttaatgagctgaaagatagagattcagaaacacgagttgatctgaaatttatgtacctggatcctccaagagatcatcacaccttagagattcagcagcaagccctgctaagagagcagcagaagaggctgaacagaataaaaatgcaggaaggtgccaaagttgacttagatgccatcccaagtgctaaagtacgagagcaaagaatgcccagagatgacactagtgatttcttgaaaaactcattattggaatctgatagtgcttttattggggcttacggtgagacatatcctgccattgaagatgacgtcctccctccaccatcacagttgccctctgcacgggagcgcaggaggaacaaatggaaaggactagacattgatagcagtcgtcctaatgtagcaccagatggtctctctctaaaatctatatccagtgtaaatgttgatgagcttagagtgagaaatgaggaacgaatgcgaagactgaatgaatttcacaataaacctattaatacagatgatgagagttcactggttgaccctgatgacatcatgaaacacataggggatgacggatcaaactctgtagcaactgagccctggctccgccctggcacttcagaaacgctgaaacgtttcatggcagagcagctgaaccaggagcagcagcagattcctggaaaaccaggcactttcacttggcagggcctgtcgactgcacatggttaaaataaacctgtactggacccagtagtgccttttaaggtgaaaggaatggtaaatctgtacctttaatatgtcctacttttggcccctacctgaaagttactttttttccatcatctgtatataaaattatttttatcatgatgtatattatgtacataaataaaaggccatgattattgatttatataatagaattgtatagattatttttgcacagttttgtcataaattagggtggtaatgaactggattgaactactatatgtgcattatattgaattctgcttgtcattaagataaggtgaataagtgtcttaaacgtcctgtaaaaccggactcccctttgttacatgcacattttccattgttacctcgatgcaaaagaattcatttagtaggtacatctattgtagctgtgattattccagtttctgtgtgatgcaatcaaatgtcctattaattaattattatttcatgtcatttgtagctactgatacagcagaaatgaagggaactgtaattacttgtatttttgtaagccatacgttaaatgtttgttacatcatctttctgcttctatttttatgccaatgaaggcatttgtcttgttactaattacatgatgtaactacttcttgatataaataaatttttattttaattact

SEQ ID NO: 56 - protein, 1185 aa, Homo sapiens
MADNLDEFIEEQKARLAEDKAELESDPPYMEMKGKLSAKLSENSKILISMAKENIPPNSQQTRGSLGIDYGLSLPLGEDYERKKHKLKEELRQDYRRYLTQERLKLERNKEYNQFLRGKEESSEKFRQVEKSTEPKSQRNKKPIGQVKPDLTSQIQTSCENSEGPRKDVLTPSEAYEELLNQRRLEEDRYRQLDDEIELRNRRIIKKANEEVGISNLKHQRFASKAGIPDRRFHRFNEDRVFDRRYHRPDQDPEVSEEMDERFRYESDFDRRLSRVYTNDRMHRNKRGNMPPMEHDGDVIEQSNIRISSAENKSAPDNETSKSANQDTCSPFAGMLFGGEDRELIQRRKEKYRLELLEQMAEQQRNKRREKDLELRVAASGAQDPEKSPDRLKQFSVAPRHFEEMIPPERPRIAFQTPLPPLSAPSVPPIPSVHPVPSQNEDLRSGLSSALGEMVSPRIAPLPPPPLLPPLATNYRTPYDDAYYFYGSRNTFDPSLAYYGSGMMGVQPAAYVSAPVTHQLAQPVVNTVGQNELKITSDQVINSGLIFEDKPKPSKQSLQSYQEALQQQIREREERRKKEREEKEEYEAKLEAEMRTYNPWGKGGGGAPLRDAKGNLITDLNRMHRQNIDAYHNPDARTYEDKRAVVSLDPNLATSNAENLEDAANKSSGHMQTQSSPFARGNVFGGPPTELQIKQQELYKNFLRFQIEEKKQREEAERERLRIAEEKEERRLAEQRARIQQEYEEEQEKKREKEEEQRLKNEEHIRLAEERQKEAERKKKEEEEKYNLQLQHYCERDNLIGEETKHMRQPSPIVPALQNKIASKLQRPPSVDSIIRSFIHESSMSRAQSPPVPARKNQLRAEEEKKNVIMELSEMRKQLRSEERRLQERLLHMDSDDEIPIRKKERNPMDIFDMARHRLQAPVRRQSPKGLDAATFQNVHDFNELKDRDSETRVDLKFMYLDPPRDHHTLEIQQQALLREQQKRLNRIKMQEGAKVDLDAIPSAKVREQRMPRDDTSDFLKNSLLESDSAFIGAYGETYPAIEDDVLPPPSQLPSARERRRNKWKGLDIDSSRPNVAPDGLSLKSISSVNVDELRVRNEERMRRLNEFHNKPINTDDESSLVDPDDIMKHIGDDGSNSVATEPWLRPGTSETLKRFMAEQLNQEQQQIPGKPGTFTWQGLSTAHG

SEQ ID NO: 57 - DNA, 4603 bp, Homo sapiens
  CDS (371)..(1435)
gtcattccattgttctgggcctgtgaagtttaccatagtgtggcatttgaagtatcatacctgtcacaatgagcattctaatctggaagagctgttccaaaaacataaacttagtgttgatgaagacttttgtcattatttgaagaatgacaactgttggacaacaaaaaatgaaaacttagattgcaacagtgattcacaggtgtttccctctttgaataataaagaactaataaatatcagaaatgtttcaaaccaggaaagatcggatgttgtagccagaacacaaaaagatgggtttcatatctttattgtttctattaaaacggagaatacagatgcaagctggaatttgaatgtttctctttctatgattgggcctcatggatatatctctgcatcagattggcccctaatgattttttacatggtgatgtgtattgtttatatattatatggcatactctggctgacgtggtctgcctgttattggaaagatatattaagaatccagttctggattgcagctgttatttttttgggaatgcttgaaaaagcagttttttatagtgaataccaaaacatcagcaacactggactgtcaacccaaggcttattgatatttgcggagttgatttctgcgattaagaggacgttggctcgccttctcgtgatcattgtgagcctgggctatggcattgtgaagcctcgtttaggaacagtcatgcaccgggtgatcggactggggcttctatacttaatctttgcagctgttgaaggcgtgatgagagtcattgggggttctaaccatttagctgttgttcttgatgacattattttagcagttattgactccatttttgtgtggttcatttttattagtttggcacaaactatgaagaccctaaggctaagaaagaacactgtgaaattttcattatatagacattttaaaaatactctgatctttgctgtgctggcttctatagtgtttatggggtggacaactaagacatttagaattgcaaaatgccaatcagattggatggaacgctgggttgacgatgcattttggagcttccttttttcgcttatccttattgtaatcatgtttttgtggagaccatcagcaaacaatcagagatatgccttcatgcccttaatagatgattctgatgatgaaattgaggaattcatggtaacttctgaaaatttaaccgaaggaataaaattaagagcctcaaaatcagtttccaatggaacagctaagcctgccacttctgagaactttgatgaagatttgaagtgggtagaagaaaatattccctcttcattcacagatgtagctcttccagtgttagtggattcagatgaggaaatcatgaccagatctgaaatggctgaaaaaatgttctcttcagaaaagataatgtgattggaacccgtataagaatgtagttaagcctgaaggactatccttcatcaagactgaaagtgagctttgatttgatattgcctaaaaatttttattgtgttatcttggaagtctgtgtatcaaaatgaagaattcagatggtaggaggttctatagtccttttaaagctgactcttgagtgtcagttgaatatccattaaattggatttggaaataacctgaggaaagtattatgataaagatctgcacagatgcctcttagctgataggtggcaggcctgtgggtttgggttctccctcttttctctggaacatatgacaattccagattaaagaaaaatgttttttaataaatacccttggtctttcttctagtcacctttgaggtagatattgtgattttctggagtatagtatatccgtgtctctgtgtcttaggtttactagatgcaataatacttctctttgacatttgtactgaagtgatttgatattaagtaaaacagttaatgtttgaatataggcatatttataggttttttccgctcccccccaacccaccctttttaaaaaatctatacaaagcccttgtttgagtctcatcatgcacatcaaatcatggagttaggtcttctctgagctcaggggaacacaagtgcacagagagagatgtcttgagggtcactaccaaagaattaccctcattgtccctcactcaggccatgtgtacatgcgatgctgctgagtgtgctggggtgggtggtggccacgtggctcccccagagcacttcctaactggcaagctgggagacccattactggtgaactttgtggaaattagaactgtatcttttacataatcttggcatattacatttcataataaaaacatacatttagttgcatgctacatcactattgattttataattaatttcttaagcttcaaccatgttttataccttatttcgttacatcatatatttgtaatgtgtaatatgaaatcttttgctttaatgtctttttttaaaatgtagaatgttctaaacttgaaaggcaattgaatgtagtatgatgaaaatgtgaatgttttgctgctttcatgaccaaagatacagggctagtggacatttagaataataattaaagctagagtcttgtatgtcttttctttgaaggagttctaaccttgtaaattgagaatgacttcagagaattttgattaagaaaacattaaaatcttaaccggcacaaacactccaatttttttcactgtgaagccgcaagcaattttttttctttttctttcaaaagcctgccttctgaatttatttcttgtttactcatttcagagagggtagtaaagaagatctatttctggtagtcatatcgcttgaaaggtattggtaaatgtgttttcagtcgtgaccatgtggaaagtgaacagtgttggcaaacattaccgagaaaatcatgcttttcaagatgcccttgctttgggatatccttcctagggagaaaaaaaaaaagtagtttaacaattgtgaattccatttcttatttcagtttctgctgcagtaatgggttcccacccactataattcccagcatttatgttctgttgtattctccccttagcccagtaacatttttatctaataccccattccccaagttttgagacagattgaccccctactcattatgtggctctagttgaattttaaaatgtggaatattgggcttgcaggcagtaggagctgcaaatctggtagagtgggagtgtggagttaatggtgagtatgttaataaagggaaactgtctctgacagaatctcagtaatgtttaccaaaacatgtctttctacagctggtaggataaatgatgctaccctgtagctcagctacaggctgcagtgcaaacttttcttccatccagagaaagcagaattccctcctagtaacctcattacaaatactgttactagaagggcatgtgctgtctgtcaccttcagtaatatttgtgccatctcttgatgactgatgacctggatcgagtatttctatgaagggtcttcttaggccccttacatacgcaagaggggtgctctagtgccatagctgtagttcacaggaaggacaccaggagaagttatacctagggctactgagcagctcatcatccctgtttctgcacagtttcctgaaactggccatcagggcctctgaggcactcaaatcagtttacttttagcatgcccccatcagggtgggtctcactgttagtgaggatacgggtctggtttgatgtttttctaggcaaaatgcttaagtgttctggttatgccattcattcatacgatgtgtgaaatttgcttaaaagggaattttcatgatttgatttagattagtatttaaatatctgctttagatagcaattaattttattgtaaaaataaggaaaaatatgtgaatatgtgaattttttaagcctgagagatgatagaatgttcccatatttttcttgtaaagaaaataatattttaacttacacatcctgtagaaaataccaccttttccccttgtattacagtacaatgtttacattactatactgtcaagctgaaagtataaaaaatgtacatatacattttgagttatgtatcctttttttaaaaaaaagttcgagtctgttgcactaggctgtacatgactaaagttgacagatgctatgctagatttataatcactagttctggtacttgtgtctttgtatgatcaaagcatgcaataagcaatacaaaataccaagccttatacttaaaagaagtttaacatattggttaatatactggttaatatactggttaaacatattgaatgtatataagtggcaaaactagatttttaaggaagtgtacattataatattggagctcagtactgcatgaagagacttcattaaaactaagaaaacatttatttggggagaaattttaggcatttaagaacttgtatttttctattttaaaaagttaaattattccgtaatttggaagaagtttcgttgaatgtaggacataaccgtttgaagggttttcatttgaaaaattgatgtattttgtgccttaatattttgttcttttaataaaaatgctctgaatttg

SEQ ID NO: 58 - protein, 354 aa, Homo sapiens
MIGPHGYISASDWPLMIFYMVMCIVYILYGILWLTWSACYWKDILRIQFWIAAVIFLGMLEKAVFYSEYQNISNTGLSTQGLLIFAELISAIKRTLARLLVIIVSLGYGIVKPRLGTVMHRVIGLGLLYLIFAAVEGVMRVIGGSNHLAVVLDDIILAVIDSIFVWFIFISLAQTMKTLRLRKNTVKFSLYRHFKNTLIFAVLASIVFMGWTTKTFRIAKCQSDWMERWVDDAFWSFLFSLILIVIMFLWRPSANNQRYAFMPLIDDSDDEIEEFMVTSENLTEGIKLRASKSVSNGTAKPATSENFDEDLKWVEENIPSSFTDVALPVLVDSDEEIMTRSEMAEKMFSSEKIM

SEQ ID NO: 59 - protein, 192 aa, Homo sapiens
MKSFLPVHTIVLIRENVCKCGYAQSQHMEGTQINQSEKWNYKKHTKEFPTDAFGDIQFETLGKKGKYIRLSCDTDAEILYELLTQHWHLKTPNLVISVTGGAKNFALKPRMRKIFSRLIYIAQSKGAWILTGGTHYGLMKYIGEVVRDNTISRSSEENIVAIGIAAWGMVSNRDTLIRNCDAEVPVGQEEVC

SEQ ID NO: 60 - protein, 242 aa, Homo sapiens
KQTELRRSGSRDVTGALLVAAAVASEAVGSLRVAEGGPNTLLLQVLRSWPWCNKELKTMEERKVKRRSPKSFSAHCTQVVNAKKNAIPVSKSTGFSNPASQSTSQRPKLKRVMKEKTKPQGGEGKGAQSTPIQHSFLTDVSDVQEMERGLLSLLNDFHSGKLQAFGNECSIEQMEHVRGMQEKLARLNLELYGELEELPEDKRKTASDSNLDRLLSDLEELNSSIQKLHLADAQDVPNTSAS

SEQ ID NO: 61 - protein, 374 aa, Homo sapiens
MRQPSPIVPALQNKIASKLQRPPSVDSIIRSFIHESSMSRAQSPPVPARKNQLRAEEEKKNVIMELSEMRKQLRSEERRLQERLLHMDSDDEIPIRKKERNPMDIFDMARHRLQAPVRRQSPKGLDAATFQNVHDFNELKDRDSETRVDLKFMYLDPPRDHHTLEIQQQALLREQQKRLNRIKMQEGAKVDLDAIPSAKVREQRMPRDDTSDFLKNSLLESDSAFIGAYGETYPAIEDDVLPPPSQLPSARERRRNKWKGLDIDSSRPNVAPDGLSLKSISSVNVDELRVRNEERMRRLNEFHNKPINTDDESPLVDPDDIMKHIGDDGSNSVATEPWLRPGTSETLKRFMAEQLNQEQQQIPGKPGTFTRCCK

SEQ ID NO: 62 - protein, 613 aa, Homo sapiens
MAEFKEKPEAPTEQLDVACGQENLPVGAWPPGAAPAPFQYTPDHVVGPGADIDPTQITFPGCICVKTPCLPGTCSCLRHGENYDDNSCLRDIGSGGKYAEPVFECNVLCRCSDHCRNRVVQKGLQFHFQVFKTHKKGWGLRTLEFIPKGRFVCEYAGEVLGFSEVQRRIHLQTKSDSNYIIAIREHVYNGQVMETFVDPTYIGNIGRFLNHSCEPNLLMIPVRIDSMVPKLALFAAKDIVPEEELSYDYSGRYLNLTVSASKERLDHGKLRKPCYCGAKSCTAFLPFDSSLYCPVEKSNISCGNEKEPSMCGSAPSVFPSCKRLTLETMKMMLDKKQIRAIFLFEFKMGRKAAETTRNINNAFGPGTANERTVQWWFKKFCKGDESLEDEERSGRPSEVDNDQLRAIIEADPLTTTREVAEELNVNHSTVVRHLKQIEKVKKLDKWVPHELTENQKNRRFEVSSSLILRNHNEPFLDRIVTCDEKWILYDNRRRSAQWLDQEEAPKHFPKPILHPKKVMVTIWWSAAGLIHYSFLNPGETITSEKYAQEIDEMNQKLQRLQLALVNRFHNQQDAENAFQEFVESQSTDFYATGINQLISRWQKCVDCNGSYFD

SEQ ID NO: 63 - protein, 387 aa, Homo sapiens
MPGHLQEGFGCVVTNRFDQLFDDESDPFEVLKAAENKKKEAGGGGVGGPGAKSAAQAAAQTNSNAAGKQLRKESQKDRKNPLPPSVGVVDKKEETQPPVALKKEGIRRVGRRPDQQLQGEGKIIDRRPERRPPRERRFEKPLEEKGEGGEFSVDRPIIDRPIRGRGGLGRGRGGRGRGMGRGDGFDSRGKREFDRHSGSDRSGLKHEDKRGGSGSHNWGTVKDELTDLDQSNVTEETPEGEEHHPVADTENKENEVEEVKEEGPKEMTLDEWKAIQNKDRAKVEFNIRKPNEGADGQWKKGFVLHKSKSEEAHAEDSVMDHHFRKPANDITSQLEINFGDLGRPGRGGRGGRGGRGRGGRPNRGSRTDKSSASAPDVDDPEAFPALA

SEQ ID NO: 64 - protein, 402 aa, Homo sapiens
MPGHLQEGFGCVVTNRFDQLFDDESDPFEVLKAAENKKKEAGGGGVGGPGAKSAAQAAAQTNSNAAGKQLRKESQKDRKNPLPPSVGVVDKKEETQPPVALKKEGIRRVGRRPDQQLQGEGKIIDRRPERRPPRERRFEKPLEEKGEGGEFSVDRPIIDRPIRGRGGLGRGRGGRGRGMGRGDGFDSRGKREFDRHSGSDRSGLKHEDKRGGSGSHNWGTVKDELTESPKYIQKQISYNYSDLDQSNVTEETPEGEEHHPVADTENKENEVEEVKEEGPKEMTLDEWKAIQNKDRAKVEFNIRKPNEGADGQWKKGFVLHKSKSEEAHAEDSVMDHHFRKPANDITSQLEINFGDLGRPGRGGRGGRGGCGRGGRPNRGSRTDKSSASAPDVDDPEAFPALA

SEQ ID NO: 65 - protein, 268 aa, Homo sapiens
MFVELNNLLNTTPDRAEQGKLTLLCDAKTDGSFLVHHFLSFYLKANCKVCFVALIQSFSHYSIVGQKLGVSLTMARERGQLVFLEGLKSAVDVVFQAQKEPHPLQFLREANAGNLKPLFEFVREALKPVDSGEARWTYPVLLVDDLSVLLSLGMGAVAVLDFIHYCRATVCWELKGNMVVLVHDSGDAEDEENDILLNGLSHQSHLILRAEGLATGFCRDVHGQLRILWRRPSQPAVHRDQSFTYQYKIQDKSVSFFCQRNVSCCSVT

SEQ ID NO: 66 - protein, 250 aa, Homo sapiens
MFVELNNLLNTTPDRAEQGKLTLLCDAKTDGSFLVHHFLSFYLKANCKVCFVALIQSFSHYSIVGQKLGVSLTMARERGQLVFLEGLKSAVDVVFQAQKEPHPLQFLREANAGNLKPLFEFVREALKPVDSGEARWTYPVLLVDDLSVLLSLGMGAVAVLDFIHYCRATVCWELKGNMVVLVHDSGDAEDEENDILLNGLSHQSHLILRAEGLATGFCRDVHGQVCRGLLGNGLLARVPVILDYIGESQA

SEQ ID NO: 67 - DNA, 2612 bp, Homo sapiens
  CDS (347)..(1276)
tgaaggaggctgtgcctccgggttgcacgaagagtccgagtcatttctcagaaggttttgataggtgggccttagaggagacgccgccgagcaccgcaagaactggaaaacacacccctctctgtctgcctgggagagccacggaaattggcacttctctgagtgaagctgaggagaaggctgtaaatctgccaaaacagccttgaagtattcttttgtcatgaggaagtgacggctgctggagggaggtgaacaccacaaggagagatggcatctggcctgggccccgcctagcagcagctccacctcctaggccaggccctgtgggatgcgccactagaccaccatggacggatcccacagcgcagccctgaagctgcagcagctgcctcccacaagtagctccagcgccgtaagcgaggcctccttctcctacaaggaaaacctgattggcgccctcttggcgatcttcgggcacctcgtggtcagcattgcacttaacctccagaagtactgccacatccgcctggcaggctccaaggatccccgggcctatttcaagaccaagacatggtggctgggcctgttcctgatgcttctgggcgagctgggtgtgttcgcctcctacgccttcgcgccgctgtcactcatcgtgcccctcagcgcagtttctgtgatagctagtgccatcataggaatcatattcatcaaggaaaagtggaaaccgaaagactttctgaggcgctacgtcttgtcctttgttggctgcggtttggctgtcgtgggtacctacctgctggtgacattcgcacccaacagtcacgagaagatgacaggcgagaatgtcaccaggcacctcgtgagctggcctttccttttgtacatgctggtggagatcattctgttctgcttgctgctctacttctacaaggagaagaacgccaacaacattgtcgtgattcttctcttggtggcgttacttggctccatgacagtggtgacagtcaaggccgtggctgggatgcttgtcttgtccattcaagggaacctgcagcttgactaccccatcttctacgtgatgttcgtgtgcatggtggcaaccgccgtctatcaggctgcgtttttgagtcaagcctcacagatgtacgactcctctttgattgccagtgtgggctacattctgtccacaaccattgctatcacagcaggtgcaatattttacctggacttcatcggggaggacgtgctgcacatctgcatgtttgcactggggtgagttctgtcctgcttgccagccccaaaacattgtgtgtagaattccaagcaggttgcccctctctctcccttattgctacagccgccccatcctttagagtcagaggagctgtggggttttggagctttgtccccagaggtgagcgttttattcctctaagggctttatcttccccttctcttttggactctaatctctttggcatttctcaaatatcacattttgacacatgaaaagtctctcaacctcacctcggatctaagaaatgcatattgaagctacatgagataccatcattttgcaaaaactaacaggattaatagtattccacattggcaagaagatgaggaggcccgcctactaccacactgttagcatacttgaatgtgggaggaaaatttaacaacagcaacaaaaaatccaaacaagcatactttttgatacagtattccattctaatcatttatcttggaaaaatgcttcaacagggatgcaaatacacatgtatgaggctatttattaatttttaaattagttgtataagcaaacaattgaaaacaatagaaatgctcagcagtagaatacaagtgttaaaaaattatgccatatccagataatagcatgccatgctgccattttgaaaacttgaggccagtctgtaggtgctgacacagatccatgtctagaacacattcatcaaaaaaaatcacaggctgggagaggtggctcacacctgtaatcccagcactttggaaggccgaggcaggtggattacctgaggtcgggagtttgagaccagcctgaccaacatggagaaaccccgtctgtactaaaaatacaaaaattagttgggcatgatggtgcatgcctgtaatcccagctactcgggaggctgaggcaggagaatcgcttgaacccgggaggcggaggttgtggtgactcgagattgcgccattgcagtccagcctgggcaacaagagcaaaattccgtctcaaaaaaaaaaaaaaaaatcacaaacttttaaaaatttggccttggccgggtgcagtggctcgtgcctataattccagcactttgggaggctgaggcgggtggatcacctgaggtcaggagtttgagtctagcctggccaacgtgctgaaaccccgtctgtactaaaaatacaaaaaaattagccaggcatggtggcaggcacctgtaatcccagctactcgggaggctgaggcaggagaatcacttgaacctgggaggtggaggttgcagtgagctgagatcatgccattgcatgcctggacaatgagagcataagtctgtctcaaaaaaaaaaaaaaaaaa

SEQ ID NO: 68 - protein, 309 aa, Homo sapiens [sequence omitted]

SEQ ID NO: 69 - DNA, 4825 bp, Homo sapiens
  CDS (161)..(388)
cgcgctgcagacgaagcccgcgcgtgatcccgctccgggtgacctcaaagcagaagctctgaattcacctctcatctgacgactgacagctgctgccaccgccagcctctgtcccttgcccaggcctgtcacacggctgcctctcagcaggggcagtagaatgaaagagggcatgtctaataacagcaccactagcatctcccaagccaggaaagctgtggagcagctaaagatggaagcctgtatggacagggtcaaggtctcccaggcagccgcggacctcctggcctactgtgaagctcacgtgcgggaagatcctctcatcattccagtgcctgcatcagaaaacccctttcgcgagaagaagttcttttgtaccattctctaactccgtgtgtgatgaaaacgcctccttttctgaccttcaaagtcccctgtagagaccatgcatgctctaagccttagggagtgagaccaacacccatccctgcccagccaacagtggccggggcttgtcttatgtttccatctgttttcttcgtggcattcaatttcatttttttccttttcattttcatgttattttcattattggcaaagaaaatcaaaatgtttatagccaaataacaaatgtgccatgtaaaagtaagtctggacttaagagtttaaaatttttaaacatcagtttccaagtttatatcatattaatacatttcagtggataatttatttaaaaaaaaaactatgcctaaatatcccttatttgtaatattcagtatcaaattagagcattttgaccaactgaaacatataacgttttcatctcctttcctggagaaagcagctgcagaggttctgtatttcctgcatttccatcatcctagtaaaaagacaacaacccacaggattggaatggttttcttttgtttttttgttgttgttgttgttttgagatggagtctcgctctgtcacccaggctggagtgcaatggcgtgacctcagctcactgcgacctccacctcctgggttcaagcgattctcctgcctcagcctcctgagtagctgggatcataggcacgcgccaccacacccagctaattttttgtatttttagtaaagacggggtttcaccatgttggtcaggctggtctcaaactcctgacctcgtgatccacccgcctcggcctcccaaagcgctgggattacaggcgtgagccaccgcgcccggccgtggaatggttttcttagcatatcctcctggggccgtggcgccccggctgtgggccgtggtggatgtcccttgtttcagtggtcctacaaaggtagatggtgggcggacaccgaagtcaacttgactgtaaaagtaccccataccgttgacgcgctgtggcagacctgtgggtgcggtctcccctctccaaatgccacctcttcctgctccctcctcttttggctcctactcagtagttccattgtgggctgcagtaatataaggacctgttctggacacagcactgggcccgctggggttttagtagggcagccccttccctgcaggagtgaggcatggtgacagcagtcccctatgtgcccccaagtcatctgagcattggtgtgcattaaggtacttaatcttccaacaataaataccataagtgcatgtctgttttattttatttatttatctttttgagacagagtcttgctctgtcgcccgggctggaatgcaatggcacaatctcagctcaccgcaacctccactcgcaggttcaagtgatcctcatgtctcagcctccctagtagctgggatgacaagcacacgccaacatgcctggctcatttttgtatttttagtagagacggggtttcaccatgtcggccaggctggtctccaactactggcctcgagtgatctgcccaccttggcctcccaaagtgctgggattgcaggtgtgagccagtgcacccggccgtatgtcttagagtgaggggaaactgaggcacaaagcgtttatgtagtttgcccaaggttacacatggtaagcagaaaaactgggatttgagggcaggcagcccggccccagagttcccttagccccctgaactgtgctaccttccaggtctacctgtttgcaggtagggagacccacaaactatcataactgcacaccgtgaagcaaacacctttgttgcagtaacccgtggcacacttcacttcccacattctccaggaggaagccgtattctggtagtttaaggaaggtgattcatggatgccccaaggaaaggctagacatcgatgaattagctcttggattgtctaagatcaacctgaaggtcctacctaaaccaaactcatatgatcccgatgaaagaggagaatacacagtgggagcagagctctttgtgtaagtttgttctattctgtttatttggaaactgcctctctccctagttctgaaagagacttgctgtgtgatagagaacaggccatttgtgcctgaaatgttctaacctgcaaattgggtttaatggtaattatcctgcctttctgtctgggaggaaacagagatgctacatagatgaaagctctgattatcatttaaaacaaataaatgtactaaatgccgtggtataatatagggaagaatggatgacttcaaagagaccatggttccgttggaggcataaaattgcctgcaaagaggcaaatgtaatttccagaccaattaatgacttgctgccctcgtaaatggatttaggacatctttgttccttgcttcttttgttaaaactcattcttagtttcattatcagctcactaataataccagtgttgttaagatgctcaaactagagcagaattgtatgctggatgtgacgctaggacctcagatcctttgatagtgcagttatgaccaggatctctttattctgtgcttcacgtcaacatcattaacccgatgaagcgaagcacaagtcataaaggcacatcagagaagccgagaaagcggggccgtacagagagcatcaaaatcaggtcagaaaaatgctgctttcctcttccaccaaaccttctccctgttgtttttacatgccccgtccctccagattcagcctccgttttgttctctctccttttccttttcctcctctcttccttaccttcccctgtgagatgcttttctgtccttttactgcacctcttccagacctatggcatttggggacgggtcacccttcctagagtgatcctgaatccagctgcctcttctttttttgtttttgtttttgtttttgagacagagtctctttctgtcgtccaggctggagtgcagtagcgcaatcttggctcactgcaacctctgcctcctgggttcaagcgattcttgtgcctcaacctcacgaggagctgggattgcaggcgcccgccaccacacccggctaatttttgtatttttagtagagacggagtttcaccatgttggccacgctggtcttgaagcccagacctcaagtgatccgcccgccttggcctcccaaagtgctgggattacaggcatgagccaccgcgcctggcctagttccctcttcttagatggagatttctgcacatgagctgtgagatccacagacatgtcactgagctcccccactctgtcagtggaagatgaatggaataaagcctgcttgcattgtggagtgcagctgtggggacagttgtcatttttgtgttttcagcttaactgtccagcatctcccctcccttcctacagcagggacatcatgccttagatataaacttagtggtgtgcagtacccaccgtggaagctgaagaagggaagtatttcctgtctctcaggcacaccccgcttccagccccataccagtcgggaagtggtcattgacctggggtggactacctggacccttgcttggggctatgagcctggagaatgtgacacaaatgaagggatgctgagactcatttcaaggaagccaccgtggtccggggttggtagtcatacagcaaggcaggtgggtagggctggcaggctgcagggatggtgtgtccactggtagcaatgtccagggatggcagggtcctgggaatgtcatttctgctccactccttggactcgctgagctgtctccgcctccacctatcttcctacagacctcccttctagttttctgtcaattctttgagccagcaaactccatccagtacattctttcttctttcatgaaagagcttgagttggatgtaaatatatatgacctaacaattccacccctaggtgtataccctacagaaatgtgtacatgtgttcatccagagacatgctctaaatcttcacaaaaacactctccataataaccccgaacaggaaagcaccccaatgcccatgttggctggataagcacattagggtatattcacacgatggaatcccagactgcaatgggaatgagctgcaactccacccccaacttggagtgtattcaccaaccctagtgttgaacgagataaggcaaaaatgcaccataggattccatttatataaagtttaaaacccagcaaaattcatccatgcggttgcaagtagagatcagtcctaagaagacagtaaccagaagcgggcatgaggtggtgcttctggggtgttctgtttcttgatctggttgccggttacctgggtgctttccgtttgtgaacattcttggagctgtacacttttgatctgggcacttttctgtgtgtatgttatacttcaataaaaagttgggctttgtgactagcaaaaaaaaaaaaaaaaaaa

SEQ ID NO: 70 - protein, 75 aa, Homo sapiens
MKEGMSNNSTTSISQARKAVEQLKMEACMDRVKVSQAAADLLAYCEAHVREDPLIIPVPASENPFREKKFFCTIL

SEQ ID NO: 71 - DNA, 1305 bp, Homo sapiens
  CDS (11)..(1222)
tgatggcgtgatgtctcacagaaagttctccgctcccagacatgggtccctcggcttcctgcctcggaagcgcagcagcaggcatcgtgggaaggtgaagagcttccctaaggatgacccatccaagccggtccacctcacagccttcctgggatacaaggctggcatgactcacatcgtgcgggaagtcgacaggccgggatccaaggtgaacaagaaggaggtggtggaggctgtgaccattgtagagacaccacccatggtggttgtgggcattgtgggctacgtggaaacccctcgaggcctccggaccttcaagactgtctttgctgagcacatcagtgatgaatgcaagaggcgtttctataagaattggcataaatctaagaagaaggcctttaccaagtactgcaagaaatggcaggatgaggatggcaagaagcagctggagaaggacttcagcagcatgaagaagtactgccaagtcatccgtgtcattgcccacacccagatgcgcctgcttcctctgcgccagaagaaggcccacctgatggagatccaggtgaacggaggcactgtggccgagaagctggactgggcccgcgagaggcttgagcagcaggtacctgtgaaccaagtgtttgggcaggatgagatgatcgacgtcatcggggtgaccaagggcaaaggctacaaaggggtcaccagtcgttggcacaccaagaagctgccccgcaagacccaccgaggcctgcgcaaggtggcctgtattggggcatggcatcctgctcgtgtagccttctctgtggcacgcgctgggcagaaaggctaccatcaccgcactgagatcaacaagaagatttataagattggccagggctaccttatcaaggacggcaagctgatcaagaacaatgcctccactgactatgacctatctgacaagagcatcaaccctctgggtggctttgtccactatggtgaagtgaccaatgactttgtcatgctgaaaggctgtgtggtgggaaccaagaagcgggtgctcaccctccgcaagtccttgctggtgcagacgaagcggcgggctctggagaagattgaccttaagttcattgacaccacctccaagtttggccatggccgcttccagaccatggaggagaagaaagcattcatgggaccactgaagaaagaccgaattgcaaaggaagaaggagcttaatgccaggaacagattttgcagttggtggggtctcaataaaagttattttccactgaaaaaaaaaaaaaaaaaaaaaaaaaaaa

SEQ ID NO: 72 - protein, 403 aa, Homo sapiens
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAFTKYCKKWQDEDGKKQLEKDFSSMKKYCQVIRVIAHTQMRLLPLRQKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGHGRFQTMEEKKAFMGPLKKDRIAKEEGA